(19) United States
(12) Patent Application Publication
(10) Pub. No.: US 2013/0273135 A1
(43) Pub. Date: Oct. 17, 2013

(54) Controlled Release Combination Biomaterials

(71) Applicant: University of Utah Research Foundation, (US)

(72) Inventors: Amanda Elaine Brooks, South Jordan, UT (US); David W. Grainger, Salt Lake City, UT (US); Paul C. Hogrebe, Salt Lake City, UT (US); Bruce G. Evans, Sandy, UT (US); David Christopher Evans, Sandy, UT (US)

(21) Appl. No.: 13/645,918

(22) Filed: Oct. 5, 2012

Related U.S. Application Data
(63) Continuation-in-part of application No. PCT/US2011/031394, filed on Apr. 6, 2011.
Application No. PCT/US2011/031394 is a continuation-in-part of application No. 12/409,261, filed on Mar. 23, 2009.
(60) Provisional application No. 61/321,216, filed on Apr. 6, 2010.
(60) Provisional application No. 61/070,638, filed on Mar. 25, 2008.

(51) Int. Cl. A61L 27/58; A61L 27/18; A61L 27/36

(52) U.S. Cl. CPC A61L 27/58; A61L 27/3608; A61L 27/18
USPC 424/426; 424/549

(57) ABSTRACT

In one aspect, the invention relates to tissue graft combination biomaterials capable of controlled release of bioactive agents or pharmaceutically active agents through a rate-controlling polymer coating encapsulating the graft material, methods for preparing same, methods of controlled release using same, and methods for treating tissue defects. This abstract is intended as a scanning tool for purposes of searching in the particular art and is not intended to be limiting of the present invention.

### CROSS REFERENCE TO RELATED APPLICATIONS

This application is a continuation-in-part of PCT patent application serial no. PCT/US2011/031394 filed Apr. 6, 2011, which itself claims priority to U.S. provisional patent application Ser. No. 61/321,216 filed Apr. 6, 2010. This application is also a continuation-in-part of U.S. patent application Ser. No. 12/409,261 filed Mar. 23, 2009 which itself claims priority to U.S. provisional patent application Ser. No. 61/070,638 filed Mar. 25, 2008. The disclosures of each of these patent applications are hereby incorporated by reference in their entireties.

### BACKGROUND

Currently, many types of bone fillers and grafting biomaterials are marketed and FDA-approved for human implant use. Commercial examples include tricalcium phosphate, calcium sulfate, hydroxyapatite, and processed cadaveric allograft human bone grafts in large pieces, croutons and morsels, particles and powder forms intended for implant and surgical use. These products provide surrogate structural support in bone defect and musculoskeletal implant sites, and act as osteoconductive agents, or biomaterial scaffolds, to facilitate bone tissue regeneration, mechanical restoration of function, healing and structural re-integration of existing tissues. A second category of bone regenerative materials are called osteoinductive agents, usually in the form of small bioactive molecules and human purified recombinant growth factors (proteins) or extracted natural protein mixtures that stimulate or induce endogenous bone formation. Examples include Bone Morphogenetic Proteins (BMPs), statins, bioactive peptides (e.g., P15), and Demineralized Bone Matrix (DBM). These osteoinductive agents can be combined with osteoconductive biomaterials carriers in attempts to provide both benefits to patients.

Current clinically approved bone filler materials are problematic in patients because they are associated with several clinical problems, including lack of effective healing and tissue regeneration, lack of vascularity, insufficient structural and mechanical properties, and a high potential for developing infections at the surgical, trauma or implant site. Consequently, where bone loss is associated with an active infection or chronic lack of healing, currently available bone fillers are not recommended. The potential risk of introducing bone graft materials into an active infection, also at implant sites, requires a two-stage surgical procedure in which the infection is first eradicated, often requiring implant retrieval and resultant trauma, followed by implant replacement and subsequent bone grafting with autologous, synthetic, or allogenic graft materials.

Active infection at implant sites in and around bones and joints, in musculoskeletal trauma sites with or without implants, and in reducing open and closed fractures with and without fixation tooling, all remain problematic due to the prolonged systemic and/or local antibiotic treatments required for reliable resolution. Currently, when an infection is present, antibiotic is delivered to implant and trauma sites and bone defects through systemic drug infusions, through locally placed but temporary bone cement carriers, and direct topical use, all of which intend to deliver sufficient antibiotic dosing to the wound site. Antibiotic bone cement carriers placed locally into wound sites (e.g., cement beads containing antibiotics) allow the antibiotics to leach from the cement over a period of weeks. Much of the loaded drug dose is unable to leach from these solid, glassy matrices over extended times due to the dense delivery matrix and lack of ready drug transport within these carriers. Additionally, typical non-degrading or thermosetting cement-loaded matrices intended to resolve wound and implant infections require two surgical operations: one for placing the cement-drug matrix into the wound site, and a second for removal of the cement after drug dose exhaustion. Presently, no commercially available permanently implanted bone fillers or synthetic or allografted bone substitutes are able to incorporate an integrated drug, growth factor, antibiotic or combination agent release scheme either for extended periods required to eliminate infection (weeks) and that also resorb after drug release to avoid a second surgery currently required for their removal.

Current techniques of delivering drugs, growth factors, and antibiotics locally into an active implant or bone infection site include the simple topical application of drug solutions, use of a drug-soaked bone graft substitute or a collagen membrane or sponge, and use of polymer bone cement loaded with antibiotic drugs, usually as a soluble drug solution or solid drug powder dispersion, directly to the wound site. Numerous studies examining the drug leaching or elution properties of bone cement have demonstrated that the greatest concentration of drug release occurs within the first 3-7 days (so-called burst effect) followed by a reduced dose, with tapering release often too low to produce reliable antimicrobial therapy. Intravenous antibiotics are delivered to patients with bone and implant infections for an average of 6 to 8 weeks. Therefore, it is beneficial to have a local antibiotic depot to release an antibiotic above the microbial killing threshold (e.g., minimal inhibitory concentration) at these sites whether in the presence or absence of an infection for a similar time of 6 weeks, to reliably clear such infections from the implant and also the surrounding tissue which can subsequently be the source of a re-seeded infection.

Another problem that occurs in both orthopedic and dental surgery, as well as trauma and implant placement, is the occurrence of infection when bone grafting is used to fill bone defects. Typically, the rate of infection is greater when a bone graft is used than when it is not used, and with implants compared to no implants. Bone graft substitutes do not have or rapidly encourage an active host blood supply and cannot be adequately perfused by host defense components (cells and antibodies) and serum-circulating antibiotics. This “dead tissue” surrogate, while acting as a structural space filler in the wound or defect site, can also serve as a perfect site for colonization, allowing infection to occur and persist.

Thus, needed are tissue regenerating and bone graft substitutes and tissue regenerating fillers with on-board antimicrobial properties that can also incorporate and release multiple drug types in controlled, programmed yet versatile ways to wound and surgical sites: antimicrobial agents alone or in tandem with osteoinductive agents or other pharmacologically active substances to produce effective tissue generation with osteoinducing agents plus microbicidal antibiotic concentrations at the local site for extended time periods (6-8 weeks), affecting both opportunistic pathogens known to colonize wound and implant sites, those already present, and those that persist despite systemic therapy.

### SUMMARY

Disclosed are combination biomaterials comprising a biocompatible, porous substrate that can be conducive to tissue regeneration (i.e., osteoconductive, neural conductive, dermal conductive); a degradable polymer membrane coated on the substrate surface; and one or more bioactive agents or pharmaceutically active agents encapsulated within the polymer, wherein the polymer has both a structure and a molecular weight selected to biodegrade over a designated time period when implanted within a subject and thereby release the bioactive agent over the time period by polymer-controlled release.

Also disclosed are methods for preparing a tissue graft combination biomaterial comprising the steps of providing a biocompatible tissue-conductive (i.e., osteoconductive) or tissue-regenerating porous substrate; combining an effective amount of a bioactive agent or pharmaceutically active agent with the substrate; and coating the substrate surface with a degradable polymer coating as a rate-controlling membrane for agent release.

Also disclosed are the products of the disclosed methods.

Also disclosed are methods of using a combination biomaterial, the method comprising the steps of producing a tissue graft combination biomaterial comprising a biocompatible, tissue-conductive (i.e., osteoconductive or other tissue) porous substrate; a degradable polymer coated on the substrate surface; and a bioactive agent or pharmaceutically active agent encapsulated by the polymer, wherein the polymer has a structure and a molecular weight selected to biodegrade over a time period when implanted within a subject and thereby release the agent over the time period.

Also disclosed are methods for treating a tissue defect or a disease, or infection within the context of the proposed combination biomaterial platform, comprising the steps of identifying a subject having a tissue defect, disease, or infection in need of treatment; providing a tissue graft combination biomaterial comprising a biocompatible, osteoconductive, porous substrate; a degradable (e.g., biodegradable, resorbable) polymer membrane coated on the substrate surface; and a bioactive agent or pharmaceutically active agent encapsulated within or by the polymer, wherein the polymer has a structure and a molecular weight selected to biodegrade over a designated time period when implanted within a subject and thereby release the agent over the designated time period after introducing the composite into a subject proximate to the tissue defect.

Also disclosed are uses of a tissue graft combination biomaterial for treating a subject having a tissue defect, the combination biomaterial comprising a biocompatible, osteoconductive, porous substrate; a degradable (e.g., biodegradable, resorbable) polymer coated on the substrate surface; and a bioactive agent or pharmaceutically active agent encapsulated by the polymer, wherein the polymer has a structure and a molecular weight selected to biodegrade over a designated time period when implanted within a subject and thereby release the agent over the designated time period required to produce a therapeutically significant effect at that site.

Also disclosed are kits comprising at least two combination biomaterials or products of disclosed methods, wherein at least two combination biomaterials comprise different bioactive or pharmaceutically active agents. Also disclosed are kits comprising at least two combination biomaterials or products of disclosed methods, wherein at least two combination biomaterials comprise different bioactive or pharmaceutically active agents that can be selected and proportioned for mixed administration to the tissue defect site to yield controlled release of at least two different bioactive agents of the same or at distinctly different amounts or doses, and also identical or distinctly different timeframes to the site to produce a unique therapeutic effect, depending on the agents and also the specific tissue regeneration, antimicrobial therapy or therapeutic effect desired.

Also disclosed are kits comprising at least two disclosed combination biomaterials or products of disclosed methods and instructions for introducing various proportions of the different combination biomaterials or implants into a subject.

Also, disclosed herein are methods of controlled release of an effective amount of at least one or more bioactive or pharmaceutically active agents in a subject comprising administering a combination biomaterial to a subject, wherein the combination biomaterial comprises a combination biomaterial substrate and a degradable polymer wherein the degradable polymer comprises one or more bioactive or pharmaceutically active agents encapsulated by the degradable polymer, wherein the one or more bioactive agents or pharmaceutically active agents are delivered to the subject over a time period of greater than one week.

Also, disclosed herein are methods of controlled release of an effective amount of at least one or more bioactive or pharmaceutically active agents in a subject comprising administering a combination biomaterial to a subject, wherein the combination biomaterial comprises a combination biomaterial substrate and a degradable polymer wherein the degradable polymer comprises one or more bioactive or pharmaceutically active agents encapsulated by the degradable polymer, wherein the one or more bioactive agents or pharmaceutically active agents are delivered to the subject over a time period of greater than one week, wherein the one or more bioactive agents or pharmaceutically active agents are delivered to the subject in a therapeutically effective amount over a time period of greater than six weeks.

Also, disclosed herein are methods of making a degradable polymer comprising dissolving a degradable polymer in a solution of a solvent for the degradable polymer at a concentration between 0 and 1000 mg/mL; heating the solution to a temperature below the boiling point of the solvent to form a heated solvent solution; adding one or more nonsolvents to the heated solution to form a heated solvent/nonsolvent solution; reducing the temperature of the heated solvent/nonsolvent solution to induce a thermodynamic phase inversion of the polymer network, thereby producing a degradable polymer.

Also, disclosed herein are methods of making a degradable polymer comprising dissolving a degradable polymer in a solution of a solvent for the degradable polymer at a concentration between 0 and 1000 mg/mL; heating the solution to a temperature below the boiling point of the solvent to form a heated solvent solution; adding one or more nonsolvents to the heated solution to form a heated solvent/nonsolvent solution; reducing the temperature of the heated solvent/nonsolvent solution to induce a thermodynamic phase inversion of the polymer network, thereby producing a degradable polymer, wherein the polymer is allowed to completely dissolve in the solvent.

Also, disclosed herein are methods of making a degradable polymer comprising dissolving a degradable polymer in a solution of a solvent for the degradable polymer at a concentration between 0 and 1000 mg/mL; heating the solution to a temperature below the boiling point of the solvent to form a heated solvent solution; adding one or more nonsolvents to the heated solution to form a heated solvent/nonsolvent solution; reducing the temperature of the heated solvent/nonsolvent solution to induce a thermodynamic phase inversion of the polymer network, thereby producing a degradable polymer, wherein the nonsolvent can be completely or partially dissolved in the heated solvent/nonsolvent solution.

Also, disclosed herein are methods of making a degradable polymer comprising dissolving a degradable polymer in a solution of a solvent for the degradable polymer at a concentration between 0 and 1000 mg/mL; heating the solution to a temperature below the boiling point of the solvent to form a heated solvent solution; adding one or more nonsolvents to the heated solution to form a heated solvent/nonsolvent solution; reducing the temperature of the heated solvent/nonsolvent solution to induce a thermodynamic phase inversion of the polymer network, further comprising adding one or more solid particulate soluble porogens to the heated solvent/nonsolvent solution.

Also, disclosed herein are methods of making a degradable polymer comprising dissolving a degradable polymer in a solution of a solvent for the degradable polymer at a concentration between 0 and 1000 mg/mL; heating the solution to a temperature below the boiling point of the solvent to form a heated solvent solution; adding one or more nonsolvents to the heated solution to form a heated solvent/nonsolvent solution; reducing the temperature of the heated solvent/nonsolvent solution to induce a thermodynamic phase inversion of the polymer network, further comprising adding one or more solid particulate soluble porogens to the heated solvent/nonsolvent solution, wherein the solid particulate soluble porogens are incorporated into the degradable polymer to create a secondary porous network within the phase-inverted micro structure.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several embodiments and together with the descriptions serve to explain the principles of the invention.

FIG. 1 is the graphical embodiment of the ‘tiered’ drug loading system on a combination biomaterial substrate (in this example, human allograft bone fragments, croutons'). This includes (1) a combination biomaterial substrate with drug ‘soak’ (drug adsorbed to substrate from solution only), or (2) a combination biomaterial substrate with drug carried within the coated degradable polymer rate-controlling release matrix, or (3) pre-micro-encapsulated drug (e.g., microparticulate solid drug formulations) loaded within the coated degradable polymer rate-controlling release matrix, or (4) bone croutons with drug carried within the coated degradable polymer rate-controlling release matrix and mixed into a demineralized bone matrix (DBM) combination biomaterial substrate, forming a drug-loaded crouto-DBM implant composite, or (5) combinations of these various strategies (example: (2)+(3); or (1)+(3)). The diverse versatility of the drug loading scheme on and within the combination biomaterial and the controlled release programming of the degradable polymer rate-controlling matrix, in addition to tunability of the rate-determining degradable polymer release coating (release barrier), all allow variable but application-specific drug loading, dosing and drug release profiles of multiple agents to be fabricated into a combination biomaterial for multiple therapeutic functions.

FIG. 2 shows in vitro gentamicin drug release profiles from different preparations of antibiotic-loaded combination biomaterial substrates (allografts) for 24 hr, 72 hr, and 1-through 6-week time points. All samples exhibit initial bolus drug release desired for initial anti-microbial therapy in the local implant-tissue environment around the combination biomaterial. Biomaterial samples without the polymer (example: degradable polycaprolactone (PCL) polymer) controlled release coating are exhausted of their drug payload essentially after 1 week and show sub-therapeutic release after a few days. Biomaterial samples with a degradable polycaprolactone (PCL) polymer controlled release coating continue to release drug well beyond 1 week. All curves are power-fit. See FIG. 3 for drug release profiles beyond the typical drug bolus release regime.

FIG. 3 depicts the adjusted timescale of FIG. 2 to highlight the degradable polymer coating-mediated drug controlled release regime of such a combination biomaterial. The 1- to 6-week time course of drug release exhibits depletion of the ‘drug soak only’ combination biomaterial (diffusive drug exhaustion without a polymer coated rate-controlling membrane) with longer-term maintenance of therapeutic levels of drug release only coming from the degradable polymer-coated samples. Groups 1 through 3 are linear fit, while Group 4 is power fit to accommodate an extended bolus release from the DBM composite carrier. This greatly enhances the drug loading capacity and control of drug release from the combination biomaterial.

FIG. 4 shows cumulative (mass-based) drug release profiles over the 6-week time course, highlighting the differential drug loading and dosing attainable through different formulation methods. Direct combination biomaterial substrate (allograft) drug soaking and DBM-base allograft composite mixing provide custom variable, high drug loading. Using a rate-controlling coated polymer membrane (i.e., degradable polymer coating) to modulate drug release yields extended dosing and controlled release while incorporating less total drug. All curves are logarithmic fit.

FIG. 5 displays the zone of inhibition (ZOI) for antibiotic released against Escherichia coli cultures on agar plates as exhibited by a combination biomaterial comprising a combination biomaterial substrate (an allograft bone morsel) packed with gentamicin-containing DBM infused throughout the bone crouton pore structure, and further coated with gentamicin-loaded within a 200,000 Da molecular weight PCL (degradable polymer). The ZOI distance for the image is 7.49 mm and the bone allograft crouton surface area in contact with agar is 59.85 mm². The ZOI was measured as the distance from the edge of the bone crouton to the perimeter of the region in which no bacterial growth film could be visibly discerned. Efficacious ZOI results for these controlled release, tailored drug loaded implants supports maintenance of therapeutic drug bioactivity throughout combination allograft fabrication and subsequent drug release.

FIG. 6 is a graph plotting zones of inhibition in bacterial agar cultures as a function of the duration of drug release as antibiotic eluted from bone allograft combination biomaterial constructs. “Drug soak-only” bone crouton samples produce no ZOI after 1 week of drug release to agar. Controlling drug release with a PCL rate-controlling coating prolongs drug release and its resulting pharmacological efficacy (ZOI) throughout the assigned 6-week therapeutic window desirable for antimicrobial efficacy in vivo post-implant surgery. Group 4 demonstrates the most potent bacterial killing capacity at all time points. Drug loading in a PCL matrix exhibits clear advantage over traditional “drug soak-only” approaches. All curves are logarithmic fit.

FIG. 7 is a graphical representation of the allograft drug loading and polymer coating degradation scheme, highlighting the flexibility in multiple bioactive or pharmaceutically active drug incorporation, and two primary end points of therapeutic enhancement: greatly minimizing the probability of infection (via locally delivered antibiotics) and facilitated, potentially accelerated orthopaedic healing (via osteoconductive combination biomaterials substrates with polymer-encapsulated osteoinductive growth factors). Additionally, FIG. 7 visualizes further the potential polymer matrix layering with combination drug delivery strategy proposed in FIG. 1. Two or more drugs can be accommodated in locally applied implant-released combination therapies using multiple drug loading and distinct controlled release schemes on a single substrate, or as a tailored mixture of single drugs on single substrates with each platform mixed in the implant site to provide both drugs at the implant site from different release-controlled substrates.

FIG. 8 is a graph of tobramycin release profiles from bone allograft preparations based off varying [1] PCL polymer coating molecular weight (here 10,000 Da versus 80,000 Da), and [2] state of drug microencapsulation within the polymer coating (example; lipid/sugar micro-encapsulated drug particles vs unencapsulated “free” drug incorporated within the rate-controlling polymer matrix). The drug amount units given are relative fluorescence intensity, correlating to a relative amount of tobramycin present as determined by a colorimetric assay quantifying tobarmycin derivatized with ortho-phthalaldehyde (OPA). Emphasis is placed on the relationship between the drug release kinetics curves that demonstrate: [A] low molecular weight PCL coatings release more drug per unit time than their higher molecular weight PCL coating analogs, and [B] drug loaded into the polymer coating in a lipid microencapsulation form (as opposed to free drug) further slows drug release from the polymer coating but to an intermediate degree when compared to changes in polymer coating molecular weight. Collectively, these results indicate that drug delivery is tunable, controlled and attainable in rate-controlled, extended release from the polymer coatings implementing this proposed ‘tiered’ formulation system for drug loading and controlled release from the tissue graft biomaterials.

FIG. 9 is a plot of data found in FIG. 8 normalized to polymer coating mass applied to bone graft biomaterials. Due to variations inherent to bench-scale spray coating and polymer deposition methods and inherent to the commercial implant-grade allograft materials geometries and sizes, it is difficult to apply precisely the same amount of polymer coating to each bone morsel. Upon normalizing the drug release data, the same trends become clearly apparent as described in FIG. 8, namely modulation of polymer coating molecular weight and drug encapsulation state affect the amounts of drug eluted per unit time in a predictable, tunable manner to provide controlled release, dosing and extended release to durations significantly beyond other reported methods.

FIG. 10 depicts a combination biomaterial substrate (here vacuum-dried allograft bone croutons) coated with tobramycin in PCL, and A) followed by an unloaded 10 kD PCL overcoat, or B) without an unloaded PCL overcoat. Arrows indicate i) the collection of polymer/drug on the surface, and ii) cracks around the allograft pore. FIG. 10 also shows methanol-treated allograft croutons coated with tobramycin/PCL, and C) an unloaded 10 kD PCL overcoat, or D) without an unloaded PCL overcoat. Arrows indicate i) the collection of polymer/drug along the outside edge of the pores and ii) collection of cracks and material around the pore. FIG. 10 also shows air-dried allograft bone crouton coated with tobramycin in PCL and in E) an unloaded 10 kD PCL overcoat, or in F) without an unloaded overcoat. Arrows indicate the varying crack sizes on the polymer coating surface.

FIG. 11 shows a comparison of the average amount of tobramycin released per processing and coating conditions of a combination biomaterial. Each graph depicts allograft fragments that have an unloaded overcoat (shaded coloring) with those lacking the overcoat (white). Different treatment conditions are shown in A (air-dried), B (methanol-treated), and C (vacuum-dried). * indicates a significant difference at α=0.05 while # indicates significance at α=0.1.

FIG. 12 shows a reaction of OPA reagent with primary amines in the presence of a sulthydryl compound (β-mercaptoethanol). One OPA molecule reacts with each primary amine. Tobramycin has 5 primary amines.

FIG. 13 shows drug OPA derivative fluorescence signals for tobramycin standards in PBS averaged over multiple runs (n=9, +SEM). Limit of detection (3 times signal: noise)=0.0625 mg/ml. The solid line indicates a linear regression from 0 to 2 mg/ml. The R value for this regression is shown. A linear regression between 0 and 8 mg/ml gives an R of 0.852 (not shown).

FIG. 14 shows a comparison of the drug OPA fluorescence assay (white bars) to drug mass spectrometry (grey bars) from the same samples.

FIG. 15 shows a comparison of tobramycin release from the PCL coating crouton cohorts using the OPA fluorescence assay over a period of 4 weeks. A) average concentration of tobramycin released was determined at 6 time points. Significant differences (a<0.1) were determined by pair wise one-way ANOVAs and grouped and indicated by an asterisk or symbol above the bar. B) average percent of tobramycin released calculated based on the measured amount of tobramycin added. C) cumulative percent of tobramycin released was calculated by adding the amounts released at each specific time point.

FIG. 16 depicts SEM imaging showing 2 different formulations A) acetone-dissolved PCL-coated allograft bone, and B) freeze-dried water-acetone mixed PCL formulation-coated allograft bone. The increased coating porosity by SEM is shown clearly in the insets of B and the cracked surface and occluded pores in A.

FIG. 17 shows a 96-well OPA-tobramycin detection assay comparing different polymer coating formulations. Certain polymer-drug formulations included 4% water non-solvent that appeared to impact tobramycin release kinetics.

FIG. 18 shows antimicrobial testing: A) Zone of inhibition in vitro antimicrobial results compared for 3 different polymer-drug formulations (10 kD PCL with 10% tobramycin, 80 kD PCL with 10% tobramycin, 80 kD PCL with 4% non-solvent water and 10% tobramycin) and B) minimal inhibitory concentrations (MIC values) compared for 3 different drug-polymer formulations out to 42 days against bacterial growth in agar. (+) indicates growth at that particular time point, (−) indicates the absence of growth, (+/−) indicates potential growth, and ND indicates that the MIC was not determined at that time point.

FIG. 19 shows that OPA reacts with one or more of the 5 primary amines of tobramycin according to the reaction shown. The spectral shift identified by HPLC shows little to no background from either OPA or underivatized tobramycin. Tobramycin gives at least 5 different peaks corresponding to the 5 primary amine derivatives.

FIG. 20 shows a comparison of different formulations using a 96-well colorimetric assay based on the OPA derivatization of tobramycin. A) 6-week time frame B) first 72 hours C) initial week.

FIG. 21 shows different concentrations of tobramycin-containing 10 kD PCL-coated (60 mg/ml and 100 mg/ml) allograft fragments provide differing release kinetics as determined via the radial diffusion of drug.

FIG. 22 shows different forms of allograft (i.e., fragments and micron-sized particulate) provide different drug release kinetics.

FIG. 23 shows that there are differences in drug release kinetics based on the addition of PEG to the polymer formulation (A). There is also a difference in kinetics based on the application method (dip-coating in a mixture of PCL and PEG vs dip coating in alternating layers of PCL and PEG) (B).

FIG. 24 shows a summary of observations during the in vivo rodent antimicrobial pilot experiments (8 mice were used in the study). The mouse shown in A) was implanted with polymer-controlled, antibiotic-releasing allograft particulate in a subdermal pocket implant infection model. There is nice hair growth, smooth coat and out-stretched appearance approximately 3 weeks post implantation. In contrast, the mouse shown in B was implanted with uncoated particulate and displays a more hunched appearance as well as a raised edematous surgical site and additional hair loss.

FIG. 25 shows an assessment of bacterial load and tobramycin presence in the urine at 24 hours post implantation.

FIG. 26 shows coated commercial ProOsteon 500R® synthetic bone graft solids in both fragment (A, C, and D) and particulate form (B). A) Comparison of drug release from pure PCL and PCL/PEG blended polymer coating drug-releasing formulations; B) Comparison of PCL/PEG coating application techniques; C) Formulations include lipid microencapsulated tobramycin and consider different application techniques; D) Comparison of allograft- and ProOsteon-coated fragments. No significant ZOI or drug release differences were identified.

FIG. 27 shows the controlled microstructure for a PCL polymer in a composite SEM image. This is distinctly different from a solid monolithic material is used either as the biomaterial substrate or as the degradable polymer controlled release coating over a substrate, or both. The slab was created using the phase inversion solvent/non-solvent methods described in this application. PCL was dissolved at 150 mg/mL with an 8% v/v addition of water as nonsolvent. Phase inverted at −20° C. and extracted with water. (A) ×50 magnification and (B) ×300 magnification.

FIG. 28 shows the differential secondary macroporous polymer structures in a composite SEM image, all derived from solid porogen incorporation into the phase inverted degradable polymer during solvent-nonsolvent processing. This microstructure-controlled biomaterial is used either as the combination biomaterial substrate or as the degradable polymer, or both. These porous polymer matrices were created using phase inversion methods associated with this application. PCL was dissolved at 150 mg/mL with an 8% v/v addition of water as nonsolvent. Phase inverted at −20° C. and extracted with water. The (A) series displays matrices using solid NaCl microparticles as the porogen species, while the (B) series highlights solid granulated glucose as the porogen loaded into the PCL during polymer coating fabrication.

FIG. 29 shows one example of a mixed solvent/non-solvent ternary phase matrix developed for PCL to create the unique degradable polymer microstructures that serve either as the combination biomaterial substrate and/or a degradable polymer that can be used as a controlled-release coating. Polymer is first dissolved in acetone, to which a selection of nonsolvent is added, either pure or in a combination of 2 or more. The resultant properties of the phase-inverted degradable polymer are a function of the thermodynamic conditions imposed on the polymer chains by a given solvent/nonsolvent system. Percentages of nonsolvent are relative to the total volume of solvent (i.e., not relative to the total mass composition).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, biomaterials, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance may or may not occur, and that the description includes instances where said event or circumstance occurs and instances where it does not.

As used herein, the term “treatment” refers to the medical management of a patient with the intent to produce a therapy, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a tissue defect” means having been subjected to a physical examination by a person of skill, for example, a physician, and found to have a condition that can be diagnosed or treated by a tissue graft combination biomaterial or other combination biomaterials described herein.

As used herein, the phrase “identified to be in need of treatment for a disorder,” or the like, refers to selection of a subject based upon need for treatment of the disorder. For example, a subject can be identified as having a need for treatment of a tissue defect (e.g., a bone defect) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a disclosed combination biomaterial to a subject. Administration can be by way of introduction of a combination biomaterial into a subject. For example, administration can be introduction via surgical implantation or injection. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired therapeutic result or to have an therapeutically significant effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. Guidance can be found in the literature for appropriate dosages for given classes of pharmaceutical products. In further various aspects, a preparation can be administered in a “prophylactic ally effective amount”; that is, an amount effective for prevention of a disease or condition.

As used herein, the term “pharmaceutically acceptable carrier” refers to both to the degradable polymer of the combination biomaterial substrate (e.g., pieces, croutons, morsels, super-micron and sub-micron particles, nanoparticles, natural polymers, synthetic polymers, ceramics, composites, and their micro structured coatings and scaffolds) that is loaded with and carrying the pharmaceutically active agent(s) and/or biologically active agent(s) on the combination biomaterial substrate, and to sterile aqueous or nonaqueous solutions, dispersions, suspensions or emulsions, as well as sterile powders or microencapsulation matrices or nanoencapsulation matrices for reconstitution into sterile injectable or coatable solutions or dispersions for incorporating pharmaceutically active agent(s) and/or biologically active agent(s) into the degradable polymer. Examples of suitable aqueous and nonaqueous carriers, diluents, solvents or vehicles include water, acetone, salines, buffers, ethanol, polyols (such as glycerol, propylene glycol, polyethylene glycol and the like), carboxymethylcellulose and suitable mixtures thereof, vegetable oils (such as olive oil) and injectable organic esters such as ethyl oleate, polymeric solubilization agents including polymer surfactant micelles, and polymer carrier solutions such as those known to produce gellable depots in tissue beds, including but not limited to Pluronics and Tetronics, PEO-PLGA-PEO block copolymers, and their biocompatible gelling block copolymers analogs. Proper fluidity can be maintained, for example, by the use of coating materials excipients such as polymer mixtures, added salts or solutes, adding non-solvents and their mixtures, or adding lipids (lecithins), by the maintenance of the required particle size in the case of microencapsulated or nanoencapsulated drug dispersions, percent loading of added excipients or salts, changing the polymer molecular weight or branching, changing drug loading, and by the use of surfactants. These compositions can also contain excipients such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and other dissolved tonicity solutes. Injectable depot forms are made by forming microencapsulated or nanoencapsulated matrices of the drug or drugs in degradable (e.g., degradable, resorbable) polymer coatings (such as polycaprolactones, polylactide-polyglycolide homo- or co-polymers, poly(orthoesters), protein-based polymers, recombinant proteins and natural proteins, poly(tyrosines), polyphosphazenes, polyphosphates and polyphosphonates, polysaccharides, proteoglycans, hyalurons, chitosans, and chondroitins, other glycoaminoglycans, starches and polysaccharides, and poly(anhydrides)) on injectable or implantable particle or solid piece dispersions of the osteoconductive biomaterial graft substrate.

Depending upon the ratio of pharmaceutically active agent(s) and/or biologically active agent(s) to degradable polymer and the nature of the particular degradable polymer or combination biomaterial substrate employed (i.e., its chemistry, hydrolytic tendencies, physical structure such as crystallinity or macrmomolecular domains, and its molecular weight), the rate of pharmaceutically active agent(s) and/or biologically active agent(s) release can be controlled and programmed for each pharmaceutically active agent(s) and/or biologically active agent(s), or for multiple pharmaceutically active agent(s) and/or biologically active agent(s).

The term “pharmaceutically acceptable carrier” also refers to such vehicles used for those injectable forms of the invention that allow the combination biomaterial as a particulate dispersion combination biomaterial to be introduced into wound, implant, defect and surgical sites. This can include dispersion of the combination biomaterial as particles within such “pharmaceutically acceptable carriers” such as DBM, platelet-rich plasma (PRP), fibrin glues, synthetic hydrogel, alginate, hyaluron, protein, and collagen gel coatings or injectable vehicles, solutions or gels of degradable polymers, starches (for example but not exclusive to CMCs and polysaccharide derivatives) or proteins (both natural and recombinant), and injectable isotonic salines common to pharmaceutical injectable formulations. Depot-injectable formulations can also be prepared by entrapping the drug(s) into lipid nanoparticles, solid nanoparticles and micropaticles, surfactant phases, liposomes or surfactant microemulsions or nanoemulsions, and depot-forming polymer-solvent carriers as drug vehicles that are compatible with body tissues, and incorporating these formulations into or within the degradable polymer coatings over the combination biomaterial substrate.

As used herein, the term “biologically active agent” or “bioactive agent” means an agent that is capable of providing a local or systemic biological, physiological, or therapeutic effect in the biological system to which it is applied. In some aspects, the bioactive agent is a growth factor. It is understood that proteins such as growth factors can be naturally sourced or recombinant. In some aspects, the bioactive agent comprises a transforming growth factor (TGF). Thus, in some aspects, the bioactive agent comprises TGF-β1, TGF-β2, or TGF-β3. In some aspects, the bioactive agent comprises a bone morphogenetic protein (BMP). Thus, in some aspects, the bioactive agent comprises BMP-2, BMP-4, BMP-6, BMP-7, BMP-13. In some aspects, the bioactive agent comprises a fibroblast growth factor (FGF). In some aspects, the bioactive agent comprises an insulin-like growth factor (IGF). Thus, in some aspects, the bioactive agent comprises IGF-I, IGF-II. In some aspects, the bioactive agent comprises a platelet-derived growth factor (PDGF). Thus, in some aspects, the bioactive agent comprises PDGF-BB. In some aspects, the bioactive agent comprises a vascular endothelial growth factor (VEGF) or its bioactive recombinant fragments. In some aspects, the bioactive agent comprises Bone-derived growth factor-2 (BDGF II). In some aspects, the bioactive agent comprises LIM mineralization protein (LMP-1). In some aspects, the bioactive agent comprises growth differentiation factor 5 (GDF-5). In some aspects, the bioactive agent comprises parathyroid hormone derivatives (PTH).

In some aspects, the bioactive agent is an osteogenic growth factor. In some aspects, the bioactive agent is osteoinductive. Osteoinductive examples include but are not limited to transforming growth factors (TGFs), bone morphogenetic proteins (BMPs), fibroblast growth factors (FGFs), parathyroid hormone derivatives (PTHs), Nell-1, statins, certain known osteoinductive peptides (e.g., P15, truncated PTHs or collagens), insulin-like growth factors (IGFs), and/or platelet-derived growth factors (PDGFs), or their respective therapeutic nucleotide transgenes.

As used herein, the term “pharmaceutically active agent” includes a “drug” or a “therapeutic agent” and means a molecule, group of molecules, complex or substance administered to an organism for diagnostic, therapeutic, preventative medical, or veterinary purposes. In the context of the disclosed combination biomaterials this term includes internally administered topical or locally released, and systemic human and animal pharmaceuticals, treatments, remedies, nutraceuticals, cosmeceuticals, biologicals, biomaterials, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term includes, but is not limited to, RNAi technologies and reagents, transgenes, protein growth factors, antimicrobials, antibiotics, microcidals, antiseptics, antifungals, antiinflammatories, anesthetics, and analgesics. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA) as genetic materials introduced to produce a desired therapeutic effect.

The terms “pharmaceutically active agent”, “drug”, “biologically active agent” or “bioactive agent” also includes the herein disclosed categories and specific examples. It is not intended that the category be limited by the specific examples. Those of ordinary skill in the art will recognize also numerous other compounds that fall within the categories and that are useful according to the invention.

As used herein, the term “osteoinduction” refers to the ability to stimulate the proliferation and differentiation of progenitor and partially differentiated cell types involved in initiating and completing bone formation and its tissue regeneration, including, but not limited to, exogenous pluripotent cells, mesenchymal MSC, satellite-derived muskuoloskeletal SDMSC, adipose-derived ADSC, induced pluripotent (iPS), and endogenously sourced stem cells (including MSCs, ADSC, SDMSC, both circulating and tissue resident). In endochondral bone formation, stem cells differentiate into chondroblasts and chondrocytes, laying down a cartilaginous ECM, which subsequently calcifies and is remodeled into lamellar bone. In intramembranous bone formation, the stem cells differentiate directly into osteoblasts, which form bone through direct endogenous mechanisms. Direct recruitment of other differentiated cell types involved in bone formation is also significant to healing, including differentiated microvascular and endothelial cells, mural cells and pericytes, osteoblasts, chondrocytes, chondroblasts, osteoclasts, and osteocytes. Osteoinduction can be stimulated by osteogenic growth factors such as those mentioned above, although some ECM proteins also drive progenitor cells toward the osteogenic phenotype.

As used herein, the term “osteoconduction” refers to the ability to stimulate the attachment, migration, and distribution of vascular and osteogenic cells within the combination biomaterial substrate. The physical characteristics that affect the graft's osteoconductive activity include porosity, pore size, and three-dimensional architecture. In addition, direct biochemical interactions between matrix proteins and cell surface receptors play a major role in the host's response to the graft material and ability to produce effective therapies in these sites.

As used herein, the term “osteogenic” refers to the intrinsic ability of a combination biomaterial to produce bone in the host site. To have direct osteogenic activity, the combination biomaterial substrate can contain or elicit cellular components that directly induce bone formation and regeneration. For example, an implanted collagen matrix pre-seeded with activated MSCs would have the potential to induce bone formation directly, without recruitment and activation of host MSC populations. Because many osteoconductive scaffolds also have the ability to bind and deliver bioactive molecules, their osteoinductive potential will be greatly enhanced. Therefore combinations of osteoconductive and osteoinductive materials and agents can be used for bone regenerative purposes with the combination biomaterial, the combination biomaterial substrate or the degradable polymer.

As used herein, the term “allograft” refers to a graft of tissue obtained from a donor of the same species as, but with a different genetic make-up from, the recipient. This term includes a non-living, non-viable, processed cadaveric tissue transplant between two humans. The combination biomaterial substrate can be an allograft.

As used herein, the term “autologous” refers to being derived or transferred from the same individual's body, such as for example an autologous bone marrow transplant. The combination biomaterial substrate can be an autologous bone marrow transplant.

As used herein, the term “autograft” refers to a graft of tissue obtained from an undamaged area of the patient or identical twin. The combination biomaterial substrate can be an autograft.

As used herein, the term “xenograft” refers to tissue or organs from an individual of one species transplanted into or grafted onto an organism of another species, genus, or family. The combination biomaterial substrate can be a xenograft.

As used herein, the term “alloplastic” material refers to material originating from a nonliving source. The term therefore includes inorganic and purely synthetic biomaterials. The combination biomaterial substrate can comprise an alloplastic material.

As used herein, the term “biomaterial” is any material, natural or man-made, that comprises whole or part of a living structure or biomedical device which performs, augments, or replaces a natural function. A “biomaterial substrate” or simply “substrate” is any material, natural or man-made, that can be implanted in a subject. A “biomaterial substrate” or “substrate” can be a biocompatible, porous or non-porous substrate. For example, a “biomaterial substrate” or “substrate” can be a bone or soft tissue graft structure. For example, suitable bone graft structures may include cartilage, cortical bone, cancellous bone, subchondral bone, and any combination of the various bone tissue types. In addition, a biomaterial substrate can be a bone-tendon-bone allograft used for ACL reconstruction and structures employed for long bone allograft tumor reconstruction. A “biomaterial substrate” or “substrate” can also be osteochondral plugs from autograft, allograft, and xenograft bone sources.

In medicine, the term soft tissue refers to tissues that connect, support, or surround other structures and organs of the body. Suitable soft tissue graft structures include, without limitation, muscles, ligaments, tendons (bands of fiber that connect muscles to bones), fibrous tissues, fat, blood vessels, nerves, and synovial tissues (tissues around joints). A “biomaterial substrate” or “substrate” can also be a soft tissue graft structure.

A “biomaterial substrate” or “substrate” can also be a synthetic polymer matrix scaffold, or any biomaterials scaffold of highly tunable interconnected pore structures formed for the purposes of filling a tissue defect, removing dead space, providing a surface that prompts host tissue regeneration and healing, or that mechanically or functionally augments or reinforces natural tissue function, Such synthetic polymer substrates can be porous resorbable polymers, for example clinically familiar polyesters, polyanhydrides, polyphosphazenes, polyphosphonates, polyaminoacids, recombinant protein-based polymers, and their copolymers. These polymers may also be applied as substrates in the form of a micro and/or microporous scaffold that may or may not display pore interconnectivity; or a monolithic polymer element such as a block, film, suture or sheet; or a collection of polymer particulates of homogenous or heterogenous size distributions.

A “biomaterial substrate” or “substrate” can also be a synthetic ceramic matrix in the form of a micro and/or microporous scaffold that may or may not display pore interconnectivity; monolithic ceramic element such as a block or sheet; or a collection of ceramic particulate of homogenous or heterogenous size distribution.

A “biomaterial substrate” or “substrate” can also be a composite ceramic and polymer matrix in the form of a micro and/or microporous scaffold that may or may not display pore interconnectivity; monolithic composite element such as a block, film, suture or sheet; or a collection of composite particulate of homogenous or heterogenous size distribution.

A “biomaterial substrate” or “substrate” can also comprise one or more bioactive agents or pharmaceutically active agents. The one or more bioactive agents or pharmaceutically active agents can be encapsulated within the biomaterial substrate in the same or similar manner in which bioactive agents or pharmaceutically active agents are incorporated into the degradable polymer.

As used herein, the term “combination biomaterial” refers to a composition of matter comprising at least two or more biomaterials. The term “combination biomaterial” includes at least one combination biomaterial substrate and at least one degradable polymer, wherein the degradable polymer comprises one or more bioactive agents or pharmaceutically active agents encapsulated by the degradable polymer. For example, a “combination biomaterial” can comprise a combination biomaterial substrate comprising a degradable polymer, wherein the degradable polymer comprises one or more bioactive agents or pharmaceutically active agents encapsulated by the degradable polymer, wherein the one or more bioactive agents or pharmaceutically active agents are delivered to the subject over a time period of greater than one week.

A “combination biomaterial” can serve as a rate-programmed drug-releasing system in combination with a biomaterial used for a purpose other than drug delivery. Thus the term “combination biomaterial” includes an implantable biomaterial serving two functions in the host: one as an implant with structural biomaterial function (for example, a graft material for substituting for bone and growing new bone), and the second as a controlled release drug delivery biomaterial to enhance the performance of this biomaterial in its context in situ (see Wu, Grainger, Biomaterials, 27, 2450-2467, 2006).

As used herein, the term “microspheres” shall mean generally spherical drug-loaded particles 1 um-100 um in size. Microspheres comprise a hollow space encapsulated by lipids, or sugars, polymers, at least one surfactant, or any combination thereof, wherein the hollow space contains therapeutic agent. As used, herein, microspheres can be used to place and release encapsulated drug within the degradable polymer membrane surrounding a combination biomaterial in order to enhance drug loading, prolong and control drug dosing and extend release of drug to more therapeutic durations.

As used herein, the term “microencapsulated” refers to the enclosure of a bioactive agent(s) or pharmaceutically active agent(s) into carrier particles of about 1 um-100 um in size. Bioactive agent(s) or pharmaceutically active agent(s) can be encapsulated by lipids, sugars, polymers, or inorganic solids, or any combination thereof, wherein the microencapsulating matrix acts to hinder drug dissolution and release. The term “nanoencapsulated” refers to this same process of coating or encapsulating bioactive agent(s) or pharmaceutically active agent(s) but is distinguished by the coated bioactive agent(s) or pharmaceutically active agent(s) being sized below 1 um, e.g., 10 nm to 1000 nm in size.

The terms “biodegradation,” “bioabsorption,” “resorption”, “degradation” and “bioerosion” are often used to connote different functional processes and definitions of biomaterial degradation, dissolution and removal from the implant site. In biodegradation, a biological agent like an enzyme, cell or a microbe is the dominant component in the degradation process. The disclosed combination biomaterials, degradable polymers or the combination biomaterial substrates can be resorbable or degradable. For example, degradable polymers are usually useful for short-term or temporary applications. Degradation and resorption imply that general hydrolytic mechanisms degrade the biomaterial. Bioresorption and bioabsorption imply that the degradation products are removed by cellular activity, such as phagocytosis, in a biological environment. By contrast, a bioerodible polymer is a water-insoluble polymer that has been converted under physiological conditions into water-soluble materials. This occurs regardless of the physical or chemical mechanism involved in the erosion process, and can include general auto-catalyzed, base or acid catalyzed hydrolysis of the polymer. Thus, where the term “degradable” is used herein, one or more of the terms “resorbable,” “degradable (e.g., biodegradable, resorbable),” “bioabsorbable,” and “bioerodable” are also disclosed.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituent.

The term “alkyl” as used herein is a branched or unbranched saturated hydrocarbon group of 1 to 24 carbon atoms, such as methyl, ethyl, w-propyl, isopropyl, w-butyl, isobutyl, s-butyl, i-butyl, w-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including optionally substituted alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as—OA¹ where A¹ is alkyl or cycloalkyl as defined above. “Alkoxy” also includes polymers of alkoxy groups as just described; that is, an alkoxy can be a polyether such as—OA 1-OA 2 or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “carboxylic acid” as used herein is represented by the formula —C(O)OH.

The term “ester” as used herein is represented by the formula —OC(O)A¹ or —C(O)OA¹ where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

The term “organic residue” defines a carbon containing residue, i.e., a residue comprising at least one carbon atom, and includes but is not limited to the carbon-containing groups, residues, or radicals defined herein above. Organic residues can contain various heteroatoms, or be bonded to another molecule through a heteroatom, including oxygen, nitrogen, sulfur, phosphorus, or the like. Examples of organic residues include but are not limited to alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

As used herein, the term “polymer” refers to a relatively high molecular weight organic compound, natural or synthetic, whose structure can be represented by a repeated small unit, the monomer (e.g., polyesters, polyamides, polyvinyls, polyanhydrides, polyorthoesters, polyaminoacids, polyalkenes, polyacrylates, polyarylates, polyolefins, polyacrylamides, polysugars, polyphosphonates, polyphosphazenes, polytyrosines, polyethers, polyurethanes, polycarbonates). Synthetic polymers are typically formed by addition or condensation polymerization of monomers. Natural polymers (biopolymers) include collagens and gelatins, silks, keratins, elastins, and their recombinant polymers and peptides, and peptide-polymer combinations, nucleic acids and their derivatives, starches including cellulose derivatives, chitosans, alginates, polyhydroxyalkanoates, glycosaminoglycans, proteoglycans, fibrin glues and fibrinogen derivatives for this purpose.

As used herein, the term “polymeric” means of, relating to, or consisting of a polymer.

As used herein, the term “copolymer” refers to a polymer formed from two or more different repeating units (monomer residues, such as degradable PLA-PLGA glycolide-co-lactide copolymers). By way of example and without limitation, a copolymer can be an alternating copolymer, a random copolymer, a block copolymer (e.g., Pluronics), or a graft copolymer. It is also contemplated that, in certain aspects, various block segments of a block copolymer can themselves comprise copolymers. These blocks can impart specific chemical and physical properties important to their use herein, such as depot forming properties in tissues as rate-limiting release barriers, control of polymer degradation, solubilization of drugs, and control of drug-particle encapsulate size (micro and nano encapsulates).

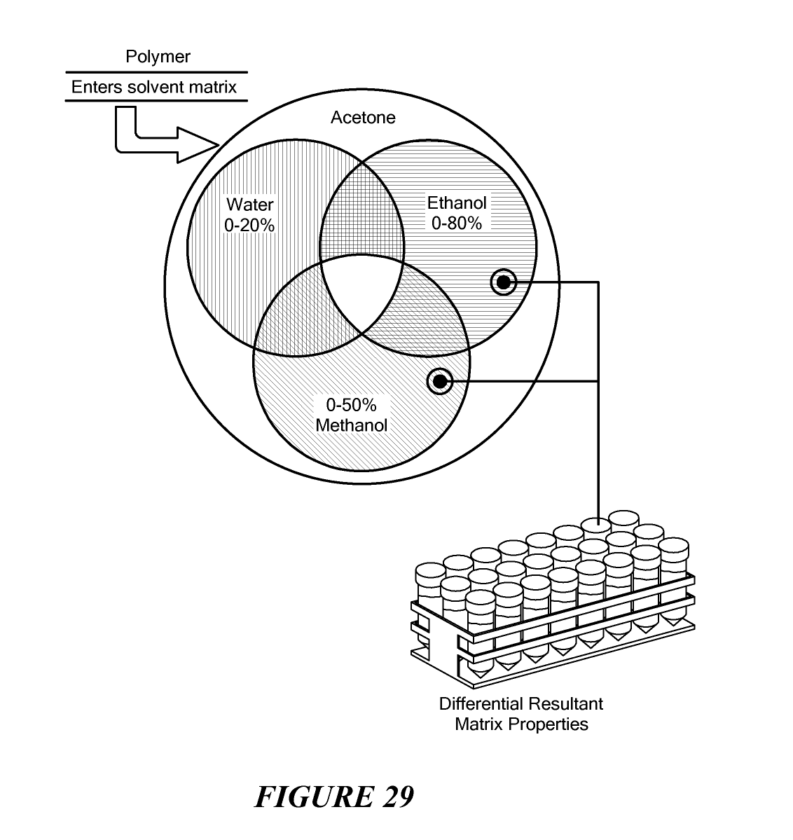

As used herein, the term “polymeric scaffold” refers to a supporting implantable matrix made of or from polymers. For example, a polymeric scaffold can be a matrix made of or from polymers with a particular shape and/or porosity, density, void fraction or microstructure. A combination biomaterial substrate can be a polymeric scaffold. A microstructure-tailored polymeric scaffold can be formed from a specific polymer solution and a non-solvent blend, for example as designated in the ternary phase diagram in FIG. 29 for PCL.

As used herein, the term “molecular weight” (MW) refers to the mass of one molecule of that substance, relative to the unified atomic mass unit u (equal to 1/12 the mass of one atom of carbon-12).

As used herein, the term “number average molecular weight” (Mₙ) refers to the common, mean, average of the molecular weights of the individual polymers. Mₙ can be determined by measuring the molecular weight of n polymer molecules, summing the weights, and dividing by n. Mₙ is calculated by:

$$Mn = \begin{matrix}{Σ}_{i} {N}_{i} {M}_{i} \\ {Σ}_{i} {N}_{i}\end{matrix}$$

wherein Ni is the number of molecules of molecular weight Mi. The number average molecular weight of a polymer can be determined by gel permeation chromatography, viscometry (Mark-Houwink equation), light scattering, analytical ultracentrifugation, vapor pressure osmometry, end-group titration, and colligative properties.

As used herein, the term “weight average molecular weight” (M_{w}) refers to an alternative measure of the molecular weight of a polymer. M_{w} is calculated by:

$$Mw = \begin{matrix}{Σ}_{i} {N}_{i} {M}_{i}^{2} \\ {Σ}_{i} {N}_{i}\end{matrix}$$

wherein Ni is the number of molecules of molecular weight Mi. Intuitively, if the weight average molecular weight is w, and you pick a random monomer, then the polymer it belongs to will have a weight of w on average. The weight average molecular weight can be determined by light scattering, small angle neutron scattering (SANS), X-ray scattering, and sedimentation velocity.

As used herein, the terms “polydispersity” and “polydispersity index” refer to the ratio of the weight average to the number average (M_{w}/Mₙ).

The term “pharmaceutically acceptable” describes a material that is not biologically or otherwise undesirable, i.e., without causing an unacceptable level of undesirable biological effects or interacting in a deleterious manner, and which allows both formulation and delivery of biologically active and pharmaceutically active agents to produce a desired therapy without clinically unacceptable effects.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural and chemical and pharmaceutical requirements for performing the disclosed therapeutic functions, and it is understood that there are a variety of structures, chemistries, materials and pharmaceutical embodiments that can perform the same function that are related to the disclosed structures, and that these structures will typically achieve the same desired result.

### B. Combination Biomaterial

Disclosed herein are combination biomaterials comprising a combination biomaterial substrate and a degradable polymer. In some aspects, the combination biomaterial comprises a combination biomaterial substrate and a degradable polymer, wherein the degradable polymer comprises one or more bioactive agents or pharmaceutically active agents encapsulated by or within the degradable polymer.

In some aspects, the combination biomaterial comprises a combination biomaterial substrate and a degradable polymer, wherein the degradable polymer comprises one or more bioactive agents or pharmaceutically active agents encapsulated by or within the degradable polymer, wherein the one or more bioactive agents or pharmaceutically active agents are delivered to the subject at therapeutic levels over a time period of greater than one week.

Disclosed herein are combination biomaterials comprising one or more agents, including bioactive agents, pharmaceutically active agents, or combinations thereof. The disclosed combination biomaterials can be implanted in a subject. In some aspects, the implanted combination biomaterials can release one or more bioactive agents or pharmaceutically active agents at the implantation site. In some aspects, the one or more bioactive agents or pharmaceutically active agents release is a controlled, extended release. Also disclosed are methods of making the disclosed combination biomaterials to select and program the rate of controlled release of one or more bioactive agents or pharmaceutically active agents, or combinations thereof, from the coated biomaterials substrate.

The combination biomaterials disclosed herein can be combination biomaterials of one or one or more bioactive agent(s) or pharmaceutically active agent(s) and one or more combination biomaterial substrates. The one or more combination biomaterial substrates can generally be selected based on the target tissue and the intended biomaterial use. For example, wherein the target tissue is bone, the combination biomaterial substrate can be a material suitable for use as a bone graft or bone filler, including but not limited to natural bone (e.g., autologous bone, allograft bone, xenograft bone), demineralized bone matrix (DBM), and alloplastic (i.e., inorganic, synthetic) graft materials (e.g., tricalcium phosphate, calcium sulfate, and hydroxyapatite, and their various physical and chemical forms, mixtures and compositions) as well as micro structured synthetic polymers as substrates.

Disclosed herein is a combination biomaterial comprising a combination biomaterial substrate; a degradable natural or synthetic polymer coated over the combination biomaterial substrate surface; and one or more bioactive agent(s) or pharmaceutically active agent(s) encapsulated by the degradable polymer matrix. The degradable natural or synthetic polymer coated over the combination biomaterial substrate surface can be a coated in a single layer, or multiple layers, wherein the degradable polymer can be the same or a different degradable polymer, and the respective solvent processing produces the tailored microstructure for each layer appropriate to control the release of each drug loaded within each layer for therapeutic purposes and at therapeutic levels beyond one week.

Disclosed herein is a combination biomaterial comprising a combination biomaterial substrate; a degradable natural or synthetic polymer impregnated into the combination biomaterial substrate; and one or more bioactive agent(s) or pharmaceutically active agent(s) encapsulated by the degradable polymer matrix.

Disclosed herein is a combination biomaterial comprising a combination biomaterial substrate; a degradable natural or synthetic polymer impregnated into and coated over the combination biomaterial substrate; and one or more bioactive agent(s) or pharmaceutically active agent(s) encapsulated by or within the degradable polymer matrix. The degradable natural or synthetic polymer coated over the combination biomaterial substrate surface can be a coated in a single layer, or multiple layers. The degradable natural or synthetic polymer coated over the combination biomaterial substrate can be the same or a different degradable polymer than the degradable polymer impregnated into the substrate, and wherein the degradable polymer can be the same or a different degradable polymer.

As disclosed herein, the degradable polymer can act as a chemical solubilizer, matrix compatibilizer, and physical carrier for loading and holding the one or more bioactive agent(s) or pharmaceutically active agent(s) in a rate-controlling membrane matrix over or on the combination biomaterial substrate, and as a rate-controlling matrix for the one or more bioactive agent(s) or pharmaceutically active agent(s) release.

By “encapsulated” is meant that the one or more bioactive agent(s) or pharmaceutically active agent(s) can be either incorporated into the degradable polymer or into or onto a combination biomaterial substrate and covered by the degradable polymer coating, such that release of the one or more bioactive agent(s) or pharmaceutically active agent(s) from the combination biomaterial is hindered and controlled by the degradable polymer coating barrier and its degradation at the site of application. Encapsulated can also refer to micro- or nano-particulate bioactive agent solid or liquid formulations incorporated into the degradable polymer coating for controlled release. Encapsulated can also refer to bioactive agents enclosed within a distinct micro- or nano-particle shells that are then incorporated into the degradable polymer coating for additional controlled release. Also as disclosed herein, one or more bioactive agent(s) or pharmaceutically active agent(s) can be further encapsulated within microspheres or nanospheres or particles prior to loading onto the combination biomaterial or into the degradable polymer coating. Thus, the one or more bioactive agent(s) or pharmaceutically active agent(s) can be both (1) microencapsulated by microspheres, nanospheres, or other agent-particle formulations, and (2) macro-encapsulated by the disclosed degradable polymer (e.g., within or beneath the degradable polymer coating), thus providing two controlled tiers for tailoring bioactive agent(s) or pharmaceutically active agent(s) agent loading, dosing and programmed release control for selected bioactive agent(s) or pharmaceutically active agent(s).

1. Substrate

In some aspects, the disclosed combination biomaterial can be used as a bone graft. Thus, in some aspects, the combination biomaterial substrate can be osteoinductive or osteoconductive. Thus, in some aspects, the combination biomaterial substrate can be allograft materials intended for skeletal and bone defect grafting and implant sites. Allgraft materials can also be DBM either loaded with bioactive agent in encapsulated or blended forms or without, and then loaded into solid bone graft porous substrates.

In some aspects, the combination biomaterial substrate comprises natural bone. In some aspects, the combination biomaterial substrate comprises bone particles, bone powder, bone putty, or a bone fragments. In some aspects, the combination biomaterial substrate comprises fragments (also referred to herein as particles, chips, morsels, croutons) of cancellous bone.

The bone can be from any suitable natural source. Thus, in some aspects, the combination biomaterial substrate comprises allograft bone. In some aspects, the combination biomaterial substrate comprises autograft bone. In some aspects, the combination biomaterial substrate comprises xenograft bone.

In some aspects, the combination biomaterial substrate comprises a synthetic or alloplastic material. For example, in some aspects, the combination biomaterial substrate comprises hydroxyapatite (U.S. Pat. No. 5,164,187). Hydroxyapatite materials can be in either a hydroxyapatite ceramic material or in a nanocrystalline hydroxyapatite form. In some aspects, the combination biomaterial substrate comprises tricalcium phosphate. In some aspects, the combination biomaterial substrate comprises medical grade calcium sulfate. In some aspects, the combination biomaterial substrate comprises gelatin or collagen gels, or proteins (recombinant or purified natural) extracted from tissues, or DBM, or a composite of suspended fibrillar collagen and a porous calcium phosphate ceramic. Providing combination biomaterial substrates are well known by those of skill in the art. The following patents are incorporated by reference in their entirety as method of teaching how to make a combination biomaterial substrate using hydroxyapatite (US Patent Application No. 2009/0048358), tricalcium phosphate (U.S. Pat. No. 6,846,853), medical grade calcium sulphate (European Patent No. 1390086), suspended fibrillar collagen and a porous calcium phosphate ceramic (European Patent No. 0243178).

Autogenous bone grafting involves harvesting the patient's own bone from a part of the body where it is not essential (typically from the pelvis or iliac crest), and transplanting it for therapeutic effect. Autogenous bone grafts are considered the gold standard due to immunologically seamless integration. Additionally, the graft has the most abundant “amount of the patient's bone growing cells and proteins” and is a kind of “outline” for repair and new bone growth. Unfortunately, this level of osteointegration requires the surgeon to make additional incisions to harvest the autologous bone graft; consequently, inflicting additional tissue trauma, postoperative pain, and surgical costs. Autologous bone is typically harvested from intra-oral sources as the chin or extra-oral sources as the iliac crest, the fibula, the ribs, the mandible and even parts of the skull.

All bone requires a blood supply. Depending on where the transplant site is and the size of the graft, an additional newly recruited blood supply may be required. For these types of grafts, extraction of the part of the periosteum and accompanying blood vessels along with donor bone is required. This kind of graft is known as a free flap graft.

Allograft bone grafting is similar to the autogenous bone graft in that the implanted graft material is still harvested from people; however, allograft bone is extracted from cadaveric bone donors; it is typically sourced from a bone bank. The bone is disinfected, deceullarized, deproteinated, and then frozen or lyophilized (freeze-dried). Allograft material minimizes problems associated with autograft material and takes the place of a bone graft extender or replacement in the procedure. Unfortunately, this type of graft is typically not very successful. It is fairly useful in several types of spinal fusions, but because it is not a very powerful “biological stimulant,” it cannot, when used as the only grafting material, typically achieve a good fusion in procedures such as a lumbar spinal fusion.

Xenograft bone combination biomaterial substitute has its origin from a species other than human, such as bovine. Xenografts are usually only distributed as a calcified matrix.

Alloplastic grafts may be made from hydroxyapatite, a naturally occurring mineral with many possible chemical and physical manifestations that is also the main mineral component of bone. They may also be made from bioactive glass. Hydroxyapatite is a synthetic bone graft, which is now the most commonly used synthetic graft due to its strong osteoconduction capabilities, hardness and bone compatibility. Calcium carbonate has also been used historically; however, its usage is starting to decrease due to its short resorption time, which leaves the resultant bone fragile. Finally, tricalcium phosphate, which now used in combination with hydroxyapatite in mixed granular and block forms, gives both effective osteoconduction and resorbability.

In some aspects, the combination biomatenal substrate is porous. Thus, in some aspects, the substrate has an average pore size of from about 100 um to about 500 um, including about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 um. In some aspects, the combination biomaterial substrate has interconnections of at least about 100 um, including at least about 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 um.

In some aspects, a degradable polymer coating or membrane is coated on and upon and contiguously over the combination biomaterial substrate surface as a rate-controlling barrier or controlled release membrane, substantially blocking the combination biomaterial substrate's pores and interconnections and communications with the ambient.

In some aspects, the combination biomaterial substrate is impregnated with a degradable polymer as a rate-controlling barrier or membrane, substantially blocking the combination biomaterial substrate's pores and interconnections and communications with the ambient.

In some aspects, a degradable polymer coating or membrane is coated on and upon and contiguously over the combination biomaterial substrate surface and impregnated with a degradable polymer, as a rate-controlling barrier or membrane, substantially blocking the combination biomaterial substrate's pores and interconnections and communications with the ambient.

The combination biomaterial substrate can also be a synthetic polymer scaffold. When the combination biomaterial substrate is a synthetic polymer scaffold, the scaffold can in some aspects release one or more bioactive agents or pharmaceutically active agents at the scaffold implantation site. In some aspects, the one or more bioactive agents or pharmaceutically active agents release is a controlled, extended release. Thus, also disclosed are methods of making the disclosed combination biomaterial substrates to select the rate of controlled release of bioactive agents, pharmaceutically active agents, or combinations thereof to produce therapy at the implant site.

The combination biomaterial substrates disclosed herein can be combination biomaterials of one or more agents and one or more combination biomaterial substrates suitable for use as scaffolds. The one or more combination biomaterial substrates can generally be selected based on the target tissue and the intended biomaterial use.

In some aspects, the disclosed combination biomaterial substrate can be a medical device, such as a stent, a sensor, a catheter, a needle, a microneedle, a fixation plate, a screw or post, a titanium, a stainless steel or a Co/Cr device for total joint replacement, or any other indwelling prosthetic medical device.

The combination biomaterial substrates can comprise, but are not limited to: polyglycolide (PGA), polylactic acid (PLA), poly(lactic-co-glycolic acid) (PLGA), polycaprolactone (PCL), other hydrolytically labile polyesters, polyurethanes (PU), polyanhydrides, polytyrosines, polyphosphazenes, polyamindo acids, recombinant protein polymers and their fragments, collagens, hyalurons, elastin-like polymers, poly ethylene glycol (PEG) and any blend or copolymer thereof.

The combination biomaterial substrates can be created, made, formed or manufactured using a variety of known methods, including the unique formulations henceforth described. For example, a degradable polymer can be dissolved into a solution of a known solvent for that polymer (example: for PCL this is acetone) at a concentration between 0 and 1000 mg/mL. The solution comprising the degradable polymer can then be heated to a temperature below the boiling point of the solvent. Next, one or more nonsolvents including, but not limited to, water, ethanol, methanol, b-butanol, n-propanol, and/or isopropanol can be added to the polymer solution. The nonsolvent can then be completely dissolved in the polymer solvent matrix. Next, heat can be applied to promote the solution. If water is used as the nonsolvent, a volume of the water at a volume to volume percentage of water to solvent of 0 to 20% is then added. Alternatively, if ethanol is used as the nonsolvent, a volume of ethanol is added at a volume to volume percentage of nonsolvent to solvent of 0 to 80%. Alternatively, if methanol is used as the nonsolvent, a volume of methanol is added at a volume to volume percentage of nonsolvent to solvent of 0 to 50%. Alternatively, if one or more of the nonsolvents described above were used, the same volume to volume percentages can be used. The next step in making the combination biomaterial substrate scaffold can require reducing the temperature of the homogenous solvent-nonsolvent(s)-polymer system so as to induce a thermodynamic phase inversion of the polymer network.

Optionally, solid particulate soluble porogens can be incorporated to create a secondary porous network within the phase-inverted micro structure of the combination biomaterial substrate. For example, metal chloride salts (NaCl, KCl, etc), phosphate salts (NaH₂PO₄, KH₂PO₄, etc), as well as glucose, alginate, agar, polyethylene glycol (PEG), wax, and/or gelatin can be used as porogens. Centrifugation can be used to promote complete packing of the porogen(s) throughout the solvent-nonsolvent(s)-polymer matrix described above. Porogens can be removed from the solvent-non-solvent solution using solvent extraction, thermal dissolution, or a combination thereof. Furthermore, the solvent and nonsolvent(s) phases can be removed using liquid extraction, evaporation at any temperature, or lyophilization.

Also disclosed herein are methods of making a degradable polymer or a combination biomaterial substrate, comprising dissolving a degradable polymer in a solution of a solvent for the degradable polymer at a concentration between 0 and 1000 mg/mL, heating the solution to a temperature below the boiling point of the solvent to form a heated solvent solution, adding one or more nonsolvents to the heated solution to form a heated solvent/nonsolvent solution, reducing the temperature of the heated solvent/nonsolvent solution to induce a thermodynamic phase inversion of the polymer network, thereby producing a degradable polymer.

The methods described herein regarding the manufacture, making or creating of combination biomaterial substrates can be used to create, form, manufacture or make blocks, sheets, discs, or otherwise planar geometry. Combination biomaterial substrates alone or in the context of a combination biomaterial can be used as a membrane, a filter, a component of a woven fabric, or for additional purposes of loading one or more bioactive agents or therapeutically active agents, or as a support for one or more layers of degradable polymer as described herein.

2. Demineralized Bone Matrix

In some aspects, the disclosed combination biomaterial further comprises demineralized bone matrix (DBM). For example, DBM can be packed into the pores of the disclosed combination biomaterial substrate. Alternatively, the polymer-coated drug-loaded combination biomaterial substrates can be dispersed and suspended as micron or smaller particulates or emulsified or encapsulated forms within DBM matrices acting as a pharmaceutical or biological agent(s) carrier. For example, bone powder sprayed with a degradable polymer coating can be mixed with DBM to form a composite paste.

In some aspects, the DBM can further comprise a bioactive agent or pharmaceutically active agent. Thus, agents can be released from the disclosed combination biomaterials from both the degradable polymer coating on the combination biomaterial substrate, and from the DBM, providing a two-phase release of different active agents.

Demineralized Bone Matrix (DBM) is the bioactive, proteinaceous constituent of processed cadaveric bone after removal of the inorganic, ceramic component. It is rich in osteoinductive signaling proteins, peptides, growth factors and cytokines, such as the BMPs. This is in addition to the collagenous extracellular matrix proteins that provide their own bioactive properties and give the paste-like material its packable characteristics and enhanced osteoinductive potential. Relevant cell types of mesenchymal lineage (namely osteoblasts) demonstrate a strong propensity to attach and migrate along collagen matrices and respond to gradients of BMPs and other osteogenic factors in such matrices.

DBM is inherently derived from cadaveric bone, making availability limited to qualified orthopaedic surgeons only. It is typically available from commercial vendors that carry allograft bone, such as Wright Medical, Synthes, etc., and is often advertised as a sister product.

DBM compositions can be prepared from multiple different DBM preparations, each of which contains DBM particles of different size and/or including different amounts or types of agents.

The disclosed combination biomaterials also provides systems and reagents for preparing and applying DBM grafts, as well as systems and reagents for treating bone defects using DBM implants. For example, the DBM composition can be provided as a paste in a delivery device such as a syringe. Preferably, the DBM composition is sterile and is packaged so that it can be applied under sterile conditions (e.g., in an operating room).

DBM can be human DBM, rat DBM, or DBM from another animal such as a cow, a horse, a pig, a dog, a cat, a sheep, or another socially or economically important animal species. In some aspects, the DBM is delipidated, such as by extraction treatment with a chloroform-methanol mixture.

3. Degradable Polymer

In one aspect, the degradable polymer can serve multiple functions in the disclosed combination biomaterials. For example, the polymer can serve as a cohesive material that facilitates drug dosing and loading, distribution and physical and chemical compatibilization by binding, stabilizing and incorporating the biologically active and/or pharmaceutically active agent(s) to the combination biomaterial substrate for eventual release. The degradable polymer can also serve as the important rate-controlling barrier mechanism for controlled release of the biologically active and/or pharmaceutically active agent(s) from the degradable polymer or from beneath the degradable polymer adjacent to the combination biomaterial substrate after introduction into a subject. In a further aspect, the degradable polymer chemical structure, physical structure of the coating (such as aggregation states with drugs, matrix coating crystallinity, and its domain morphology), and/or degradable polymer molecular weight and degradation mechanisms and rates can be selected to serve these functions, offering a level of tunability for controlling and extending drug release not possible with other technologies.

In several aspects, suitable degradable polymers can be obtained commercially. For example, various polycaprolactone formulations can be obtained from Solvay Chemicals or Lactel (Pelham, Ala.) or Sigma Aldrich in St. Louis, Mo. (Catalog numbers 440752, 440744). Many other degradable polymers for biomedical use are commercially available in other polymer chemistries.

In further aspects, those of skill in the art can readily prepare degradable polymers and copolymers by many different radical initiations, ring-opening, or condensation or recombinant, vector-based synthesis of monomers corresponding to the desired degradable polymer residues.

It is understood that the degradable polymer can be provided as a solution, emulsion or suspension in a solvent or with surfactant stabilization, for example, during spray coating or dip coating.

In some aspects synthetic degradable polymers can be used. The following are examples of synthetic degradable polymers: (including but not exclusive to) polyesters, polyamides, polyvinyls, polyanhydrides, polyorthoesters, polyaminoacids, polyalkenes, polyacrylates, polyarylates, polyolefins, polyacrylamides, polysugars, polyphosphonates, polyphosphazenes, polytyrosines, polyethers, polyurethanes, polycarbonates.

In some aspects natural degradable polymers can be used. The following are examples of natural polymers: (including but not exclusive to) collagens and gelatins, silks, keratins, elastins, and their recombinant polymers and peptides, and peptide-polymer combinations, nucleic acids and their derivatives, starches including cellulose derivatives, polysaccharides, alginates, polyhydroxyalkanoates, glycosaminoglycans, proteoglycans, fibrin glues and fibrinogen derivatives.

The disclosed degradable polymers can be created, made, formed or manufactured using a variety of known methods. For example, degradable polymers can be created, made, formed or manufactured using the methods described above for making combination biomaterial substrates.

a. Structure

In some aspects, the degradable polymer comprises monomer residues, wherein at least about 50% of the monomer residues have a structure represented by a formula:

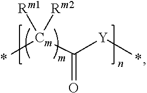

wherein m is an integer from 1 to 12; wherein n is an integer selected to yield a molecular weight of the polymer from about 5 kD to about 450 kD, including from about 5 kD to about 300 kD, from about 5 kD to about 200 kD, and from about 5 kD to about 100 kD; wherein Y is O or N—R, wherein R is hydrogen, optionally substituted alkyl, or optionally substituted aryl; and wherein each of R^{m1} and R^{m2} is independently hydrogen, halogen, hydroxyl, nitrile, nitro, thiol, optionally substituted amino, and optionally substituted organic residue. As used herein, “kD” refers to kilodaltons, or 1000 Daltons, or 1000 grams/mo.

It is contemplated that such structures can be represented such that, if m is 1, m1 represents 11, and R^{m1}=R, ¹1¹1. Likewise, if m is 2, m1 represents 11, m2 represents 21, m3 represents 31, and R¹¹, R¹², R²¹, and R²² are present in the residue structure. Thus, wherein m=3, a polymer can comprise a residue having a structure represented by a formula:

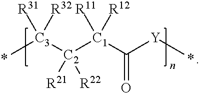

In some aspects, at least about 50% of monomer residues in the degradable polymer have a structure represented by a formula:

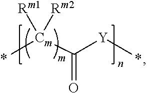

wherein m is an integer from 2 to 8; wherein n is an integer selected to yield a molecular weight of the polymer of from about 10 kD to about 450 kD, including from about 10 kD to about 300 kD, from about 10 kD to about 200 kD, and from about 10 kD to about 100 kD; wherein Y is O or N—R, wherein R is hydrogen, optionally substituted alkyl comprising from 1 to 6 carbons, or optionally substituted aryl comprising from 1 to 6 carbons; and wherein each of R^{m1} and R^{m2} is independently hydrogen, halogen, hydroxyl, nitrile, nitro, thiol, optionally substituted amino, and optionally substituted organic residue comprising from 1 to 6 carbons.

In some aspects, at least about 75% of monomer residues in the degradable (e.g., degradable, resorbable) polymer have a structure represented by a formula:

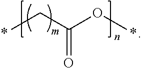

wherein m is an integer from 2 to 8; and wherein n is an integer selected to yield a molecular weight of the polymer of from about 10 kD to about 80 kD.

In some aspects, at least about 75% of monomer residues in the degradable polymer comprise caprolactone residues. In some aspects, the degradable polymer of the disclosed combination biomaterial is a polyester. In some aspects, the polyester is polycaprolactone. In some aspects, the polyester further comprises polylactic acid, polyglycolic acid and/or D, L-polylactide-co-glycolide (PLGA). In some aspect, the degradable polymer can be selected from a diverse, recognized set of biomaterials, including DBM, platelet-rich plasma (PRP), fibrin glues, nucleic acids, alginates, hydrogels, gelatins and collagen gel coatings, recombinant protein-like polymers, degradable polymers such as polyanhydrides, polytyrosines, polyaminoacids, polyphosphonates, polyorthoesters, polysaccharides and chitosans, glycosaminoglycans, starches (for example but not exclusive to CMC), hyaluronic acids or chondroitins, heparins or proteins (albumin, fibrinogen, silk, collagen and many others).

In various aspects, each of R group (e.g., R^{m1} and R^{m2}) can be independently hydrogen, halogen, hydroxyl, nitrile, nitro, thiol, optionally substituted amino, and optionally substituted organic residue.

In some aspects, the degradable polymer has a structure and a molecular weight selected to degrade over a time period when implanted within a subject and thereby release the agent(s) over the designated therapeutic time period. In some aspects, the time period is at least about one day. In some aspects, the time period is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 days. In some aspects, the time period is at least about one week. In some aspects, the time period is at least about two weeks. In some aspects, the time period is at least about three weeks. In some aspects, the time period is at least about four weeks. In some aspects, the time period is at least about five weeks. In some aspects, the time period is at least about six weeks. In some aspects, the time period is at least about seven weeks. In some aspects, the time period is at least about eight weeks. In some aspects, the time period is greater than one week. In some aspects, the time period is 1, 2, 3, 4, 5, 6, 7, 8 weeks.

b. Molecular Weight

When referring to molecular weight, it is understood that those of skill in the art typically refer to number average molecular weight (Mₙ) or weight average molecular weight (M_{w}).

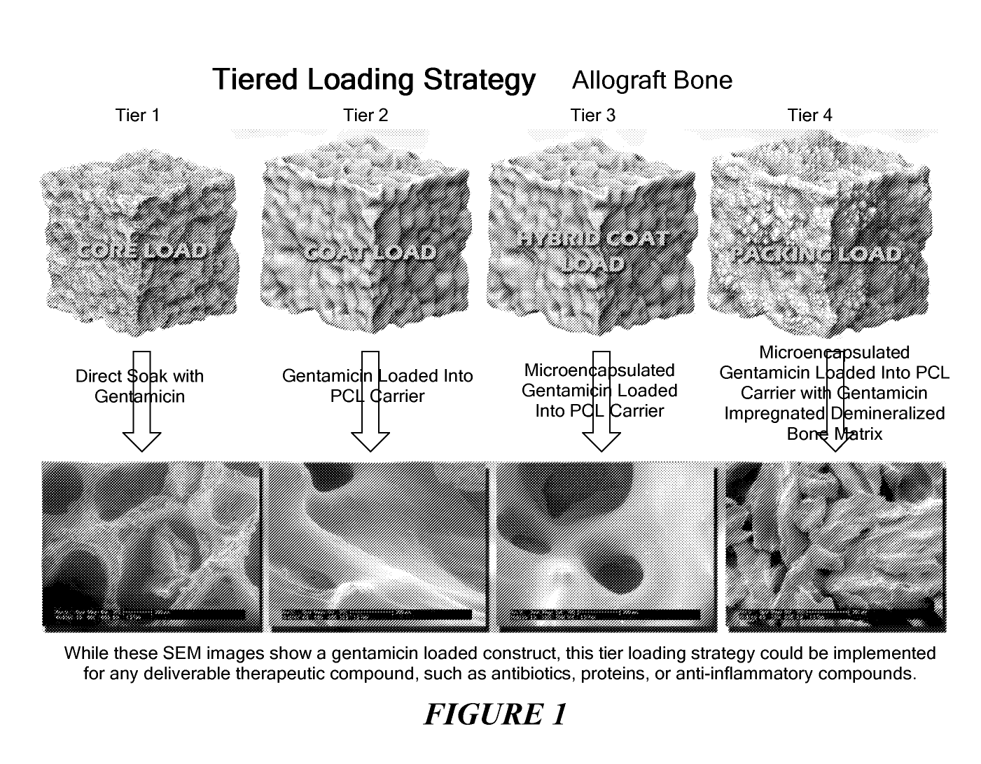

As disclosed herein, the rate of release can be selected by modulating the molecular weight of the disclosed degradable polymer. Thus, in some aspects, the combination biomaterials provide a “tier 2” release as shown in FIG. 1. Thus, in some aspects, the degradable polymer has a molecular weight of from about 5 kD to about 450 kD. Thus, in some aspects, the degradable polymer has a molecular weight of from about 5 kD to about 300 kD. Thus, in some aspects, the degradable polymer has a molecular weight of from about 5 kD to about 200 kD. Thus, in some aspects, the degradable polymer has a molecular weight of from about 2 kD to about 100 kD. In some aspects, the degradable polymer has a molecular weight of from about 5 kD to about 100 kD. In some aspects, the degradable polymer has a molecular weight of from about 10 kD to about 80 kD. In some aspects, the degradable polymer has a molecular weight of about 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200 190, 200, 210, 220, 230, 240, 250, 260, 270, 280, 290, 300, 310, 320, 330, 340, 350, 360, 370, 380, 390, 400, 410, 420, 430, 440, 450 kD.

4. Microencapsulation

In some aspects, the one or more bioactive agents or pharmaceutically active agents is/are encapsulated (e.g., microencapsulated, nanoencapsulated) as particle formulations (e.g., microspheres, nanospheres) prior to being macroencapsulated by degradable polymer coating (i.e., loaded within or beneath the polymer coating). These encapsulated particle bioactive agents or pharmaceutically active agents forms can then be loaded onto the combination biomaterial substrate by direct adsorption, impregnated into the combination biomaterial substrate using a viscous matrix such as DBM, by suspension within the degradable polymer coating, and in combinations with free un-encapsulated bioactive agents or pharmaceutically active agents. Thus, in some aspects, the combination biomaterials provide a further desired and designed “tier 3” loading and release capability as shown in FIG. 1. Particle encapsulation involves packaging an active ingredient (e.g., a bioactive agent(s) or pharmaceutically active agent(s)) inside a solid-phase capsule ranging in size from about one micron to several millimeters for microencapsulation and from about 10 nm to about 1000 nm for nanoencapsulation. The solid encapsulate matrix capsule protects the bioactive agent(s) or pharmaceutically active agent(s) from its surrounding environment until an appropriate time when the solid allows its release through various mechanisms. Then, the bioactive agent(s) or pharmaceutically active agent(s) escapes through the capsule wall by various means, including hydrolysis and rupture, enzymatic degradation, carrier dissolution, melting or diffusion to be released.

In some aspects, the bioactive agent(s) or pharmaceutically active agent(s) microencapsulated in a microcapsule or microsphere. U.S. Pat. No. 6,224,794 is incorporated by reference herein in its entirety for its teaching of how to make and use microspheres for microencapsulation of bioactive agent(s) or pharmaceutically active agent(s). Degradable (e.g., biodegradable, resorbable) microcapsules, containing one or more bioactive agent(s) or pharmaceutically active agent(s) can be prepared by methods known in the art (see Microencapsulation: Methods and Industrial Application, ed. by Simon Benita, Marcel Dekker, Inc. New York, 1996, which is hereby incorporated by reference in its entirety for its teaching of how to microencapsulate bioactive agent(s) or pharmaceutically active agent(s)). Particularly useful are microcapsule formulations which are stable at pH levels below about 9 and which lyse or release at pH above about 9. By controlling these pH variations, bioactive agent(s) or pharmaceutically active agent(s) release will be controlled at and limited to the site where apatite is being formed. Hydrolytically and dissolution controlled release from microencapsulated forms can also be used with slowly dissolving or degrading capsule solids as the matrix. U.S. Pat. No. 6,716,450 is incorporated by reference herein for the teaching of fabrication methods and properties for nanocapsules useful for encapsulating bioactive molecules such as proteins and drugs. These nanocapsules comprise branched or hyperbranched polymers and copolymers and have a core-shell structure forming a capsule volume appropriate for complexing and retaining growth factors and other bioactive molecules. The nanoencapsulated bioactive molecule is stable in extreme temperatures and pH, soluble in aqueous or organic solvents, and can be lyophilized to a dry powder for long-term storage without loss of activity.

5. Agents

The terms “pharmaceutically active agent”, “drug”, “biologically active agent” or “bioactive agent” include the herein disclosed categories and specific examples. It is not intended that the category be limited by the specific examples. Those of ordinary skill in the art will recognize also numerous other compounds that fall within the categories and that are useful according to various embodiments of the invention.

The bioactive agent(s) or pharmaceutically active agent(s) of the herein disclosed compositions and methods can be any such agent suitable for administration to a tissue graft site. In some aspects, the bioactive agent or pharmaceutically active agent is selected to promote tissue graft incorporation, promote tissue regeneration, prevent infection, or any combination thereof. For example, the bioactive or pharmaceutically active agent(s) can act to: control infection and inflammation; enhance cell growth and tissue regeneration; control tumor growth; act as an analgesic or anesthetic; promote anti-cell attachment; enhance bone growth; hinder osteoporosis; hinder fibrosis, enhance neovascularization, enhance neural proliferation, enhance cell migration, act as a chemoattractant, and enhance local anabolic or metabolic tissue functions, among other functions. Bioactive agents include prodrugs, which are agents that are not biologically active when administered but, upon administration to a subject are converted to bioactive agents through metabolism, enzymes or some other mechanism. Additionally, any of the compositions of the invention can contain combinations of two or more bioactive and pharmaceutical agents.

In some aspects, the bioactive agent or pharmaceutically active agent is present in the combination biomaterial in an amount necessary to provide a therapeutically effective dosage delivered locally to the volume around the combination biomaterial substrate over at least one week as controlled release formulation. In some aspects, the bioactive agent or pharmaceutically active agent is present in the combination biomaterial in an amount necessary to provide a therapeutically effective dosage over at least two weeks. In some aspects, the bioactive agent or pharmaceutically active agent is present in the combination biomaterial in an amount necessary to provide a therapeutically effective dosage over at least three weeks. In some aspects, the bioactive agent or pharmaceutically active agent is present in the combination biomaterial in an amount necessary to provide a therapeutically effective dosage over at least four weeks. In some aspects, the bioactive agent or pharmaceutically active agent is present in the combination biomaterial in an amount necessary to provide a therapeutically effective dosage over at least five weeks. In some aspects, the bioactive agent or pharmaceutically active agent is present in the combination biomaterial in an amount necessary to provide a therapeutically effective dosage over at least six weeks. In some aspects, the bioactive agent or pharmaceutically active agent is present in the combination biomaterial in an amount necessary to provide a therapeutically effective dosage over at least seven weeks. As disclosed herein, this extended agent release beyond the conventional initial bolus release of less than one week has specific therapeutic advantages for patient use.

In some aspects, the bioactive agent or pharmaceutically active agent is present in the combination biomaterial in an amount necessary to provide a therapeutically effective dosage over at least one day. In some aspects, the bioactive agent or pharmaceutically active agent is present in the combination biomaterial in an amount necessary to provide a therapeutically effective dosage over at least 1, 2, 3, 4, 5, 6, 7, 8, 9, 10, 11, 12, 13, 14, 15, 16, 17, 18, 19, 20 days. In some aspects, the bioactive agent or pharmaceutically active agent is present in the combination biomaterial in an amount necessary to provide a therapeutically effective dosage over at least one week. In some aspects, the bioactive agent or pharmaceutically active agent is present in the combination biomaterial in an amount necessary to provide a therapeutically effective dosage over at least two weeks. In some aspects, the bioactive agent or pharmaceutically active agent is present in the combination biomaterial in an amount necessary to provide a therapeutically effective dosage over at least three weeks. In some aspects, the bioactive agent or pharmaceutically active agent is present in the combination biomaterial in an amount necessary to provide a therapeutically effective dosage over at least four weeks. In some aspects, the bioactive agent or pharmaceutically active agent is present in the combination biomaterial in an amount necessary to provide a therapeutically effective dosage over at least five weeks. In some aspects, the bioactive agent or pharmaceutically active agent is present in the combination biomaterial in an amount necessary to provide a therapeutically effective dosage over at least six weeks. In some aspects, the bioactive agent or pharmaceutically active agent is present in the combination biomaterial in an amount necessary to provide a therapeutically effective dosage over at least seven weeks. In some aspects, the bioactive agent or pharmaceutically active agent is present in the combination biomaterial in an amount necessary to provide a therapeutically effective dosage over at least eight weeks.

It is understood that an agent can be used in connection with administration to various subjects, for example, to humans (i.e., medical administration) or to animals (i.e., veterinary administration).

Bioactive agents or pharmaceutically active agents (“agents”) include the herein disclosed categories and specific examples. It is not intended that the category be limited by the specific examples. Those of ordinary skill in the art will recognize also numerous other compounds that fall within the categories and that are useful according to the invention.

In some aspects, the bioactive agent is a growth factor. It is understood that proteins such as growth factors can be naturally sourced or recombinant. Other growth factor mimics (e.g., statins, NMP) can be synthetic small molecules or fragments of natural growth factors (e.g., VEGF fragments, collagen fragments like P15, PTH fragments). In some aspects, the bioactive agent is an osteogenic growth factor. In some aspects, the bioactive agent comprises a transforming growth factor (TGF). Thus, in some aspects, the bioactive agent comprises TGF-β1. Thus, in some aspects, the bioactive agent comprises TGF-β2. Thus, in some aspects, the bioactive agent comprises TGF-β3. In some aspects, the bioactive agent comprises a bone morphogenetic protein (BMP). Thus, in some aspects, the bioactive agent comprises BMP-2. Thus, in some aspects, the bioactive agent comprises BMP-4. Thus, in some aspects, the bioactive agent comprises BMP-6. Thus, in some aspects, the bioactive agent comprises BMP-7. Thus, in some aspects, the bioactive agent comprises BMP-13. In some aspects, the bioactive agent comprises a fibroblast growth factor (FGF). In some aspects, the bioactive agent comprises an insulin-like growth factor (IGF). Thus, in some aspects, the bioactive agent comprises IGF-I. Thus, in some aspects, the bioactive agent comprises IGF-II. In some aspects, the bioactive agent comprises a platelet-derived growth factor (PDGF). Thus, in some aspects, the bioactive agent comprises PDGF-BB. In some aspects, the bioactive agent comprises a vascular endothelial growth factor (VEGF). In some aspects, the bioactive agent comprises Bone-derived growth factor-2 (BDGF II). In some aspects, the bioactive agent comprises LIM mineralization protein (LMP-1). In some aspects, the bioactive agent comprises growth differentiation factor 5 (GDF-5). In some aspects, the bioactive agent comprises parathyroid hormone derivatives (PTH).

In some aspects, the bioactive agent or pharmaceutically active agent is an anti-inflammatory agent. Anti-inflammatory compounds include the compounds of both steroidal and non-steroidal structures. Suitable non-limiting examples of steroidal antiinflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desoxymethasone, desoxycorticosterone acetate, dexamethasone, dichlorisone, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene)acetate, flurandrenolone, hydrocortisone acetate, hydrocortisone butyrate, methylprednisolone, triamcinolone acetonide, cortisone, flucetonide, fludrocortisone, difluorosone diacetate, fluradrenolone, fludrocortisone, difluorosone diacetate, fluocinolone, fluradrenolone acetonide, medrysone, amcinafel, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting example of non-steroidal anti-inflammatory compounds include celecoxib, nimesulide, apasone, gold, oxicams, such as meloxicam, and CP-14,304; the salicylates, such as aspirin, disalcid, benorylate, trilisate, safapryn, and solprin; the acetic acid derivatives, such as diclofenac, furofenac, acematacin, zomepirac, clindanac, oxepinac, and ketorolac; the fenamates, such as mefenamic, meclofenamic, flufenamic, niflumic, and tolfenamic acids; the propionic acid derivatives, such as fenoprofen, indopropfen, pranoprofen, miroprofen, tioxaprofen, alminoprofen, and tiaprofenic; and the pyrazoles, such as phenylbutazone, feprazone, azapropazone, and trimethazone.

Anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Momifhimate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolic Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium).

In some aspects, the bioactive agent or pharmaceutically active agent is an antibiotic. Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Thus, the bioactive agent or pharmaceutically active agent can be ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxaciUin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, timidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol, or any combination thereof.

In some aspects, the bioactive agent or pharmaceutically active agent is an anti-microbial peptide. Thus, the bioactive agent or pharmaceutically active agent can comprise defensin, cathelicidin, or saposin peptides and their related derivatives. Thus, the bioactive agent or pharmaceutically active agent can comprise an antimicrobial small molecule. Thus, the bioactive agent or pharmaceutically active agent can comprise benzoxazine, bipyridinium, cyanine, guanidone, naphthalimide, nitrofuran, quinazolindiamine, quinolamine, salicylanilide, or furanone or any combinations thereof.

In some aspects, the bioactive agent or pharmaceutically active agent is an anti-septic agent. Thus, the bioactive agent or pharmaceutically active agent can comprise medical alcohols (ethanol, isopropanol), chlorhexidine and related bi- and poly-guanides (e.g., PHMB), povidone iodine, triclosan and its derivatives, and cationic antiseptics including benzylakylammonium compounds, quaternary ammonium antibiotics, and antimicrobial polycations and related compounds known for anti-septic properties.

In some aspects, the bioactive agent or pharmaceutically active agent is a therapeutic antibody drug or antibody-derivative drug class agent. Thus, the bioactive agent or pharmaceutically active agent can comprise known and emerging antibody drugs as described in Diibel, Stefan (ed.), Handbook of Therapeutic Antibodies, January 2007, 1190 pages, 3 volumes, ISBN-10: 3-527-31453-9, known to produce specific, novel therapies against osteoporosis, inflammation, tumors, infection, and also promote tissue and vascular regeneration by activating novel receptor signaling pathways in tissues.

In some aspects, the bioactive agent or pharmaceutically active agent is an osteoporosis drug such as a bisphosphonate. Thus, the bioactive agent or pharmaceutically active agent can comprise alendronate, risedronate, etidronate, ibandronate, pamidronate, zoledronate, and related compounds.

In some aspects, the bioactive agent or pharmaceutically active agent is a pro-angiogenic agent to promote therapeutic wound site angiogenesis, endothelial cell recruitment, vascular perfusion and neovascularization. Thus, the bioactive agent or pharmaceutically active agent can comprise angiogenesis promoters such as VEGF, its truncated forms and analogs, Endothelin-1, Ang-1 and -2, PDGF isoforms, and other bioactive compounds in this regard as described U.S. Pat. No. 6,284,758.

In some aspects, the bioactive agent or pharmaceutically active agent is an angiogenesis inhibitor, or anti-neoplastic or anti-tumor agent. Thus, the bioactive agent or pharmaceutically active agent can comprise any of a number of known anti-cancer drugs.

In some aspects, the bioactive agent or pharmaceutically active agent is a statin. Thus, the bioactive agent or pharmaceutically active agent can comprise lovastatin, pravastatin, simvastatin, atorvastatin, rosuvastatin, fluvastatin, and related statin derivatives.

In some aspects, the bioactive agent or pharmaceutically active agent is a transgenic bioactive molecule. Thus, the transgenic bioactive molecule can comprise a protein or peptide (e.g., an enzyme, a cytokine, a structural protein such as collagen, an antibody or other protein comprising an antibody binding site, a hormone, a detectable protein such as green fluorescent protein, a chimeric or fusion protein, a protein having a general systemic metabolic function, such as factor VIII, a virus such as a vector, etc.), a nucleic acid (e.g., a ribozyme, an antisense molecule, an aptamer, an siRNA, etc.) or a combination (e.g., a virus). Suitable bioactive molecules can further comprise compounds that cannot be encoded genetically, such as compounds or agents that prevent infection (e.g., antimicrobial agents and antibiotics), compounds or agents that reduce inflammation (e.g., anti-inflammatory agents), compounds that prevent or minimize adhesion formation, such as oxidized regenerated cellulose (e.g., INTERCEED and SURGICEL, available from Ethicon, Inc.), glycoproteins, glycosaminoglycans (e.g., heparin sulfate, heparin, chondroitin sulfate, dermatan sulfate, keratan sulfate, hyaluronic acid), analgesics, and compounds or agents that suppress the immune system (e.g., immunosuppressants). In one aspect, the transgenic bioactive molecule can comprise at least one of bound cytokines and free cytokines. In some aspects, the bioactive agent or pharmaceutically active agent is a synthetic and natural small molecules such as quorum sensing inhibitors including, but not limited to, furanones and acyl-homoeserine lactones.

In some aspects, the bioactive agent or pharmaceutically active agent is a matrix-enhancing agent. In this aspect, the matrix-enhancing molecules serve to promote the increased production of ECM to induce production of matrix proteins such as, for example and without limitation, glycoproteins, elastin, and collagen, without substantially increasing cell proliferation. Thus, the bioactive agent or pharmaceutically active agent can comprise matrix-enhancing molecules (e.g., TGF-.beta, angiotensin II, insulin-like growth factors, ascorbic acid).

In some aspects, the bioactive agent or pharmaceutically active agent is a biological matrix material. In these aspects, the biological matrix material can comprise, for example and without limitation, collagen, elastin, and fibronectin.

### C. Methods of Making the Combination Biomaterials

In one aspect, the invention relates to methods of preparing a combination biomaterial disclosed herein. In some aspects, the method comprises the steps of providing a biocompatible, osteoconductive, porous substrate; combining an effective amount of one or more bioactive agent(s) or pharmaceutically active agent(s) with the substrate; and coating the combination biomaterial substrate surface with a degradable polymer.

In some aspects, a degradable polymer can be added or introduced to a combination biomaterial scaffold by soaking the combination biomaterial substrate in a degradable polymer solution comprising the one or more bioactive agents or pharmaceutically active agents.

In some aspects, one or more bioactive agents or pharmaceutically active agents can be introduced directly into the combination biomaterial substrate. In some aspects one or more bioactive agents or pharmaceutically active agents can be introduced directly into the combination biomaterial substrate and the combination biomaterial substrate can be coated with one or more layers of one or more degradable polymers comprising one or more bioactive agents or pharmaceutically active agents. The combining and coating steps can be performed substantially simultaneously by mixing a solvated degradable polymer with both free bioactive agents or pharmaceutically active agents and microencapsulated nanoencapsulated bioactive agents or pharmaceutically active agents pre-formulations, and coating the mixture on the surface of the combination biomaterial substrate. In some aspects, the combining and coating steps are performed substantially simultaneously by soaking the combination biomaterial substrate in drug solution, impregnating the combination biomaterial substrate with the free or encapsulated drug, and coating the degradable polymer containing either free or encapsulated bioactive agent or pharmaceutically active agent particles or both over the combination biomaterial substrate.

For example a combination biomaterial substrate can serve as a scaffold and can be (1) soaked directly with one or more bioactive agents or pharmaceutically active agents (free or microencapsulated), (2) coated with a rate-controlling biodegradable, degradable or resorbable polymer coating (e.g., polycaprolactone), which can further contain a one or more bioactive agents or pharmaceutically active agents formulation (free, microencapsulated or nanoencapsulated, or suspended in a secondary degradable polymer), and/or (3) impregnated or packed into the combination biomaterial substrate with a synthetic or natural degradable polymer (e.g., DBM, PRP, PEG, hyaluron, cellulose, synthetic hydrogel collagen, fibrin glue, or other protein gel) throughout the porous structure of a combination biomaterial substrate that can also be loaded with one or more bioactive agents or pharmaceutically active agents in various physical and chemical forms. These three bioactive or pharmaceutically active agents loading and dosing levels can be referred to as the Primary Loading and Dosing Tiers.

Primary Loading and Dosing Tier (1) can be obtained by soaking a combination biomaterial directly in a solution of either free or microencapsulated bioactive or pharmaceutically active agents. Microencapsulation of the bioactive or pharmaceutically active agent can add an additional level of loading and controlled release, and hence another tier to the combination biomaterial system. The combination biomaterial substrate could then be further treated with either a both Primary Loading or a Dosing Tier (2) or Dosing Tier (3).

Primary Loading and Dosing Tier (2) can be obtained through a degradable polymer application strategy as described herein and can incorporate various bioactive or pharmaceutically activeagent formulations into the Tier (2) rate-controlling element itself. The bioactive or pharmaceutically active agent can be free in the matrix, formulated within an interspersed microencapsulated or nanoencapsulated phase, or incorporated into a secondary degradable polymer with a degradation rate different than that of the bulk rate modulating Tier (2) degradable polymer. Both microencapsulation or nanoencapsulation of the bioactive or pharmaceutically active agent and incorporation into a secondary differentially-degradative degradable polymer add additional levels of controlled release, and hence additional tiers to the combination biomaterial system. Combination biomaterial substrates treated with Primary Loading and Dosing Tier (2), can also have previously been loaded under (1) and may go on to be loaded under (3), but does not necessarily require any previous or further loading; any combination is possible.

Primary Loading and Dosing Tier (3) can be obtained by packing a void space of the combination biomaterial substrate with either synthetic or naturally derived degradable polymer (e.g., DBM, PRP, PEG, collagen or protein or polymer gel or carrier) containing one or more bioactive agents or pharmaceutically active agents. In some aspects, portions or pieces of a combination biomaterial substrate can be mixed with the Tier (3) degradable polymer to make a packable paste. For non-porous combination biomaterial substrates, the Tier (3) can be applied as a uniform surface coat. One or more bioactive agents or pharmaceutically active agents can be added to the Tier (3) in any of the forms previously described. The one or more bioactive agents or pharmaceutically active agents can be free in the matrix, formulated within an interspersed microcapsule phase, or incorporated into a secondary degradable polymer with a degradation rate different than that of the bulk rate modulating a Tier (3) degradable polymer. Both microencapsulation or nanoencapsulation of the one or more bioactive agents or pharmaceutically active agents and incorporation into a secondary differentially-degradative degradable polymer can add additional levels of controlled release, and hence additional tiers to the combination biomaterial system. Combination biomaterial substrates treated with Primary Loading and Dosing Tier (3), can have previously been loaded under (1) and/or (2), but does not necessarily have to have received any previous loading; any combination is possible.

As disclosed herein, the combination biomaterials and combination biomaterial substrates disclosed herein can provide multi-tiered nature of the bioactive or pharmaceutically active agent loading strategy, allowing for versatile tailoring of the bioactive or pharmaceutically active agent selection, combination therapies, individual bioactive or pharmaceutically active agent loadings and dosings, and controlled and extended release to the site of application to produce application- and even patient-specific treatment approaches. It is contemplated that the combination biomaterial substrates can be seeded or otherwise loaded as desired at the time of or before implantation.

In some aspects, the degradable polymer has a structure and a molecular weight selected to degrade over a therapeutic time period when implanted within a subject and thereby release the one or more bioactive agents or pharmaceutically active agents over a corresponding time period by degradation controlled kinetics.

In some aspects, the rate-controlling degradable polymer coating is spray coated. In some aspects, the coating is applied via soaking in solvent/non-solvent solutions or dip-coating methods as is common to the biomedical industry. In some aspects, the mixing and coating steps are performed substantially simultaneously. In further aspects, the mixing and coating steps are performed sequentially.

Also disclosed herein are the products produced by the disclosed methods.

### D. Kits

In one aspect, the invention relates to kits comprising at least two combination biomaterials disclosed herein, wherein the at least two combination biomaterials comprise different bioactive or pharmaceutically active agents that can be mixed to tailor appropriate balances of each agent type in the site.

Also disclosed is a kit comprising at least one combination of biomaterial disclosed herein and instructions for introducing the combination biomaterial into a subject. The combination biomaterial of the disclosed kits can comprise a degradable (e.g., biodegradable, resorbable) polymer having at least about 75% caprolactone residues. For example, the combination biomaterial of the disclosed kits can comprise a degradable (e.g., biodegradable, resorbable) polymer having at least about 75% caprolactone residues. The combination biomaterial of the disclosed kits can include one or more bioactive or pharmaceutically active agent(s). For example, the combination biomaterial of the disclosed kits can include one or more antimicrobial agents.

### E. Methods of Using the Combination Biomaterials

Also provided is a method of use of a disclosed combination biomaterials. In one aspect, the method of use is directed to the treatment of a disorder. In a further aspect, the disclosed compounds can be used as single agents or in combination with one or more other bioactive or pharmaceutically active agents in the treatment, prevention, control, amelioration or reduction of risk of the aforementioned diseases, disorders and conditions for which the compound or the other bioactive or pharmaceutically active agents have utility, where the combination of bioactive or pharmaceutically active agents together are safer or more effective than either bioactive or pharmaceutically active agents alone. The other bioactive or pharmaceutically active agent(s) can be administered by a route and in an amount commonly used contemporaneously or sequentially with a disclosed compound. When a disclosed compound is used contemporaneously with one or more other bioactive or pharmaceutically active agents, a pharmaceutical composition in unit dosage form containing such bioactive or pharmaceutically active agents and the disclosed compound is preferred. However, the combination therapy can also be administered from multiple bioactive or pharmaceutically active agents loaded on the same combination biomaterial substrate, or from mixing different combination biomaterial substrates each containing different bioactive or pharmaceutically active agents. It is also envisioned that the combination of one or more active ingredients and a disclosed compound can be more efficacious than either as a single bioactive or pharmaceutically active agents.

Disclosed herein is a method for introducing a combination biomaterial, the method comprising the steps of providing a combination biomaterial comprising: a biocompatible, osteoconductive, porous substrate; a degradable (e.g., biodegradable, resorbable) polymer coated on the substrate surface; and one or more bioactive agent(s) or pharmaceutically active agent(s) encapsulated by (e.g., sealed by, within or beneath) the degradable polymer; and introducing the combination biomaterial into a subject. In some aspects, the bioactive agent(s) or pharmaceutically active agent(s) are applied either as a direct soak (tier 1 release) to be encapsulated (e.g., sealed by, within or beneath) the degradable polymer coating, or drug captured within the degradable polymer coating (tier 2 release). In some aspects, the bioactive agent(s) or pharmaceutically active agent(s) is microencapsulated in a microsphere or nanosphere and further encapsulated ((e.g., sealed by, within or beneath) the degradable polymer coating (tier 3 release). In some aspects, all three tiers can be used on the same or different combination biomaterial substrates, alone or in mixtures, in applications. In some aspects of the method, the degradable polymer has a structure and a molecular weight selected to degrade over a designated time period when implanted within a subject and thereby release the bioactive or pharmaceutically active agent(s) over the equivalent time period by degradation-controlled polymer coating-mediated release.

Also disclosed is a method for treating a tissue defect, comprising the steps of: identifying a subject having a tissue defect in need of treatment; providing a combination biomaterial comprising: a biocompatible, osteoconductive, porous substrate; a degradable and resorbable polymer coated on the substrate surface; and one or more bioactive agent(s) or pharmaceutically active agent(s) encapsulated by (e.g., sealed by, within or beneath) the degradable polymer; and introducing the combination biomaterial into a subject proximate to the tissue defect. In some aspects of the method, the degradable polymer has a structure and a molecular weight selected to act as both a drug solubilizer and carrier, and also as a rate-controlling barrier for drug release with specified degradation capacity thereby releasing the bioactive or pharmaceutically active agent over a designated or therapeutic time period correlated to polymer degradation or alteration in vivo.

The combination biomaterial can be any bioactive combination biomaterial disclosed herein. Thus, in some aspects, the combination biomaterial releases the bioactive or pharmaceutically active agent(s) over an about one- to about eight-week period. In some aspects, the bioactive or pharmaceutically active agent(s) is an antimicrobial agent. In some aspects, the subject is a mammal. In some aspects, the subject is a human. In some aspects, introduction is surgical implantation. In some aspects, introduction is injection.

Also disclosed herein is a use of a combination biomaterial for treating a subject having a tissue defect, the combination biomaterial comprising: a biocompatible, osteoconductive, porous combination biomaterial substrate; a degradable polymer coated on the combination biomaterial substrate surface; and one or more bioactive agent(s) or pharmaceutically active agent(s) encapsulated within or by the degradable polymer, wherein the degradable polymer has a structure and a molecular weight selected to degrade (e.g., biodegrade and/or resorb) over a time period when implanted within a subject and thereby release the one or more bioactive agent(s) or pharmaceutically active agent(s) over the same time period controlled by the polymer degradation or coating alterations in vivo. The combination biomaterial can be any combination biomaterial disclosed herein. In some aspects, the disclosed use is for treating a tissue defect in a subject. In some aspects, the disclosed use is for releasing a bioactive or pharmaceutically active agent(s) in a subject.

It is understood that the disclosed methods can be used in connection with the disclosed compounds, compositions, kits, and uses.

The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment. In some aspects of the disclosed methods, the subject has been identified with a need for treatment prior to the administering step. In one aspect, a subject can be treated prophylactically with a compound or composition disclosed herein, as discussed herein elsewhere.

### F. Experimental

The following examples are put forth so as to provide those of ordinary skill in the art with a complete disclosure and description of how the compounds, compositions, articles, biomaterials and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following examples. Starting materials and the requisite intermediates are in some cases commercially available, or can be prepared according to literature procedures or as illustrated herein.

1. Antibiotic Release Profiles from PCL Matrices

a. Construction of Combination Biomaterial Substrate

Allograft cancellous croutons were cut to uniform size and weighed. Tobramycin powder was commercially microencapsulated utilizing lipid-sprayed microspheres. Six grams of polycaprolactone (PCL) were first dissolved in 150 milliliters of acetone at 47 degrees centigrade. Gentamicin powder and microencapsulated tobramycin were mixed with PCL in solution with sonication. About 6.25 mg of antibiotic was used for each bone specimen. A pressurized fine spray was used to apply the PCL solution directly to the cancellous for uniform application. Air drying for 2 hours allowed the acetone to dissipate, creating a thin coat on the bone. DBM was loaded with 40 mg/ml of gentamicin in phosphate buffered saline using syringe infusion and packed into the PCL-coated croutons for one test group.

b. Elution Phase

Four test groups (cohorts) were created, each with eight specimens. Group 1 (Control) comprised of bone croutons soaked in gentamicin solution (50 mg/ml) for 12 hrs. Group 2 (Gent) comprised of bone croutons sprayed with PCL (MW 200,000)/gentamicin solution (−50 mg/ml). Group 3 (Micro) comprised of bone croutons sprayed with PCL/microencapsulated tobramycin solution. Group 4 (DBM) comprised of bone croutons sprayed with PCL/microencapsulated tobramycin solution packed with gentamicin infused DBM (40 mg/ml). PCL is made from the following commercial process:

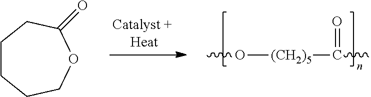

Simulated Body Fluid (SBF) was prepared according to: T. Kokubo, H. Kushitani, S. Sakka, T. Kitsugi and T. Yamamuro, “Solutions able to reproduce in vivo surface-structure changes in bioactive glass-ceramic A-W”, J. Biomed. Mater. Res., 24, 721-734 (1990). The following salt ions were present at the corresponding mM concentrations in 18 S2 Millipore treated water: Na⁺, [142.0]; K⁺, [5.0]; Mg²⁺, [1.5]; Ca²⁺, [2.5]; Cl⁻, [148.8]; HCO₃⁻, [4.2]; HPO₄²⁻, [1.0]; SO₄²⁻, [0.5].

Eight trials were completed on each of the four test groups by placing each specimen in 5 ml of simulated body fluid (SBF) and measuring drug release into SBF at 37° C. All release fluids for all specimens were exchanged at each of the following time intervals below: 24 hours, 72 hours, 1 week, 2 weeks, 3 weeks, 4 weeks, 5 weeks, 6 weeks.

c. Antibiotic Assay

Cobas Integra Therapeutic Drug Monitoring was used wherein Rl represents an antibody reagent, anti-gentamicin or anti-tobramycin monoclonal antibody (mouse), in buffer, pH 7.5, with stabilizer and preservative. R2+SR represents the tracer reagent, fluorescein-labeled gentamicin derivative in buffer, pH 8.5, with stabilizer and preservative. Fluorescence polarization was used for the quantitative determination of drug concentrations in simulated body fluid for the purpose of drug monitoring.

d. Bacterial Suppression Study

Thirty two agar petri dishes were set up with blood agar medium. Escherichia coli was used as the inoculum. 0.4 ml of inoculum was spread over the surface of each agar plate using rolling glass beads. Cancellous crouton specimens (labeled 1-32) were placed in the center of the agar-containing petri dishes. All dishes were kept in an upright position until the inoculum was absorbed (about 10 minutes). All dishes were then incubated for 45-48 hours at 37° C. Zone of inhibition was then recorded for each sample petri dish and the measurement was recorded as (1) distance of clear space from the edge of bone in mm and (2) the radius of the bone graft.

e. Release Profiles

Release profile experiments were performed as follows: cancellous bone chips treated to the prescribed regimens were allowed to release drug into a 5 ml volume of simulated body fluid (SBF) for the eight assigned time points out to 6 weeks. Release fluid was replaced at every time point and saved for drug quantification analysis using FPIA (Fluorescence Polarization Immunoassay).

f. Results

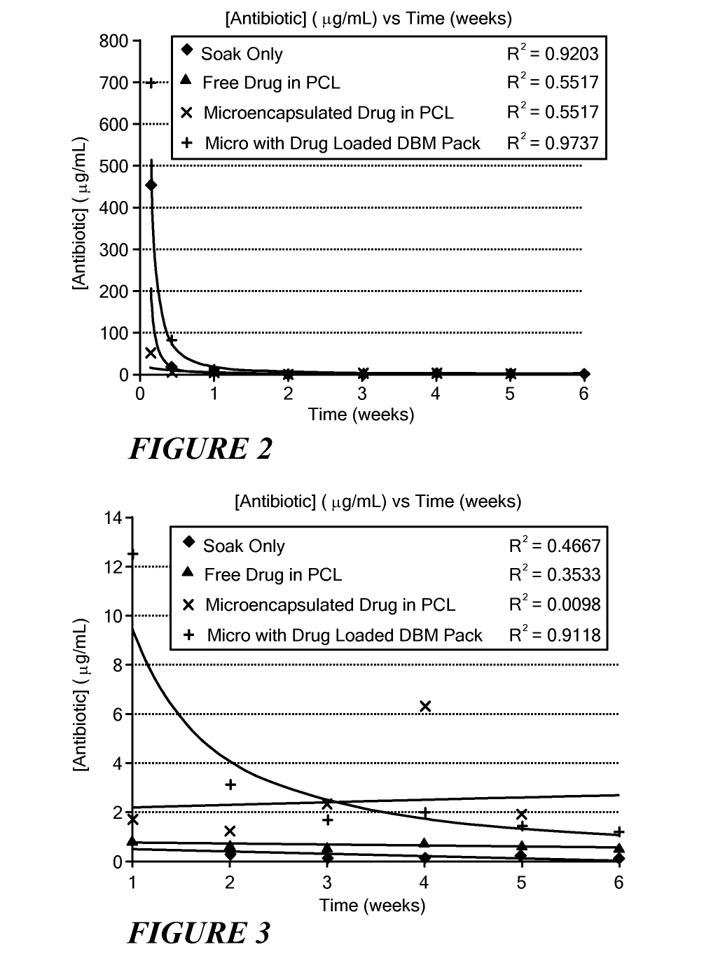

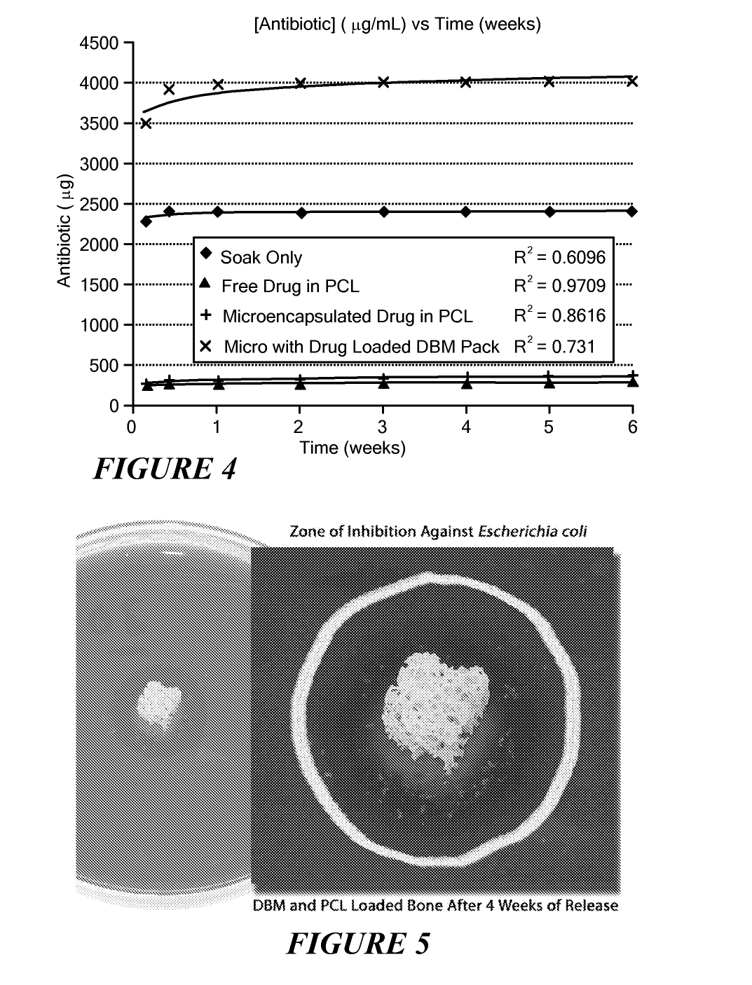

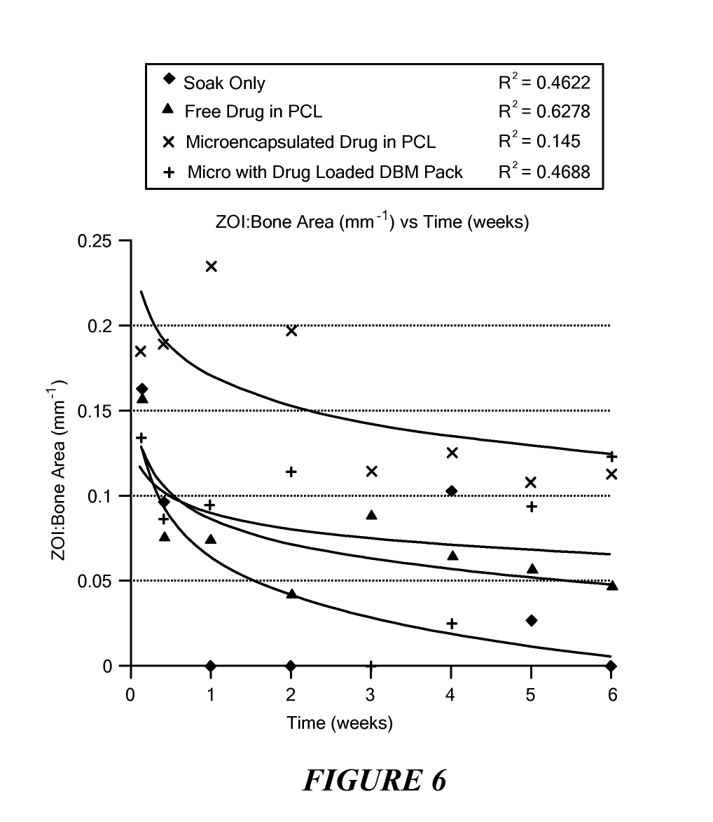

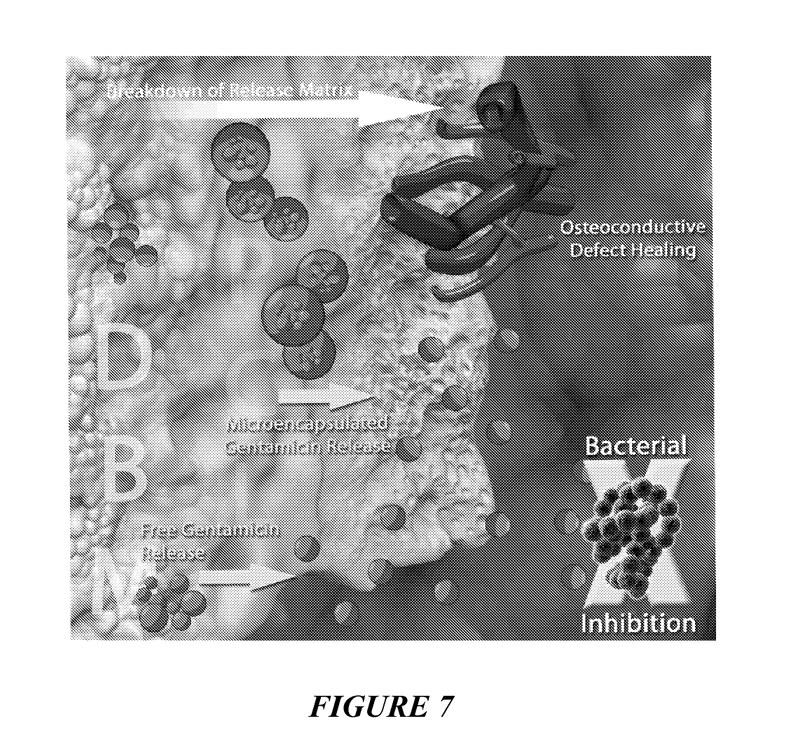

The present invention discloses an antibiotic loading strategy for allograft bone grafts that fills bone defects in both clinical infections and surgical sites. The disclosed construct uses a layered degradable resorbable polycaprolactone synthetic polymer coating over an allograft bone crouton infused with demineralized bone matrix (DBM) (tier 4 release, FIG. 1). Microencapsulated gentamicin is incorporated into the degradable polymer coating to facilitate a controlled and extended local release of the antibiotic at the site of placement (FIG. 1). The experimental elution tests demonstrated not only an effective early bolus release, but also a sustained release over 6 weeks (FIGS. 2-4). The prolonged effect of this drug release will ensure that sufficient amounts of antibiotic are present to inhibit microbial growth and prevent recurrent pathologies, such as biofilms and development of antibiotic-resistant microbes around the release site. Furthermore, functional testing with bacterial inhibition studies also provided excellent bacterial suppression both at early and late time intervals (FIGS. 5-6). Ultimately, critical structural, space filling, osteoinductive, and conductive properties of the clinically accepted bone filler material were maintained with these new antimicrobial properties (FIG. 7).

The multi-level, tunable release of antibiotic from this combination biomaterial is a powerful anti-infective attribute of bone filler applications which is currently unavailable. The disclosed construct could be applied to a large number of foot and leg bone infections associated with diabetes, in addition to orthopedic and dental infections. Perhaps the greatest demand for an antibiotic-releasing bone filler would be in a sterile surgical site with associated bone loss, implant use, revision surgeries, critical bone defects, osteosarcoma resections, and trauma injury. Antibiotic delivering bone graft will provide local bactericidal drug concentrations to prevent infections not provided by prophylactic intravenous antibiotics or other bone grafting materials commonly used today.

2. Tobramycin Release Profiles from Combination Biomaterials

a. Construction of Graft

Allograft cancellous croutons were cut to uniform size and weighed. Tobramycin powder was commercially microencapsulated utilizing lipid spray microspheres. PCL was dissolved at 60 mg/ml in 47 degrees centigrade acetone. Tobramycin powder and microencapsulated tobramycin were mixed with solvated PCL solution. Either free or microencapsulated tobramycin was mixed to the liquid PCL solution. 2 mg of antibiotic was used for each bone specimen. For uniform application, a pressurized fine spray was used to apply the PCL solution directly to the cancellous croutons as it rotated axially on a stationary ring stand. Air drying for 2 hours allowed the acetone to dissipate, creating a thin coating on the bone graft material.

b. Release Profiles

Release profile experiments were performed as follows: cancellous bone chips treated to the four prescribed regimens, [(1) 10,000 mol. wt. PCL, free tobramycin, (2) 10,000 mol. wt. PCL, microencapsulated tobramycin, (3) 80,000 mol. wt. PCL, free tobramycin, and (4) 80,000 mol. wt. PCL, microencapsulated tobramycin] were allowed to release drug into a 5 ml volume of simulated body fluid (SBF) for the eight assigned time points out to 6 weeks. Release fluid was replaced at every time point and samples saved for drug quantification analysis.

c. Results

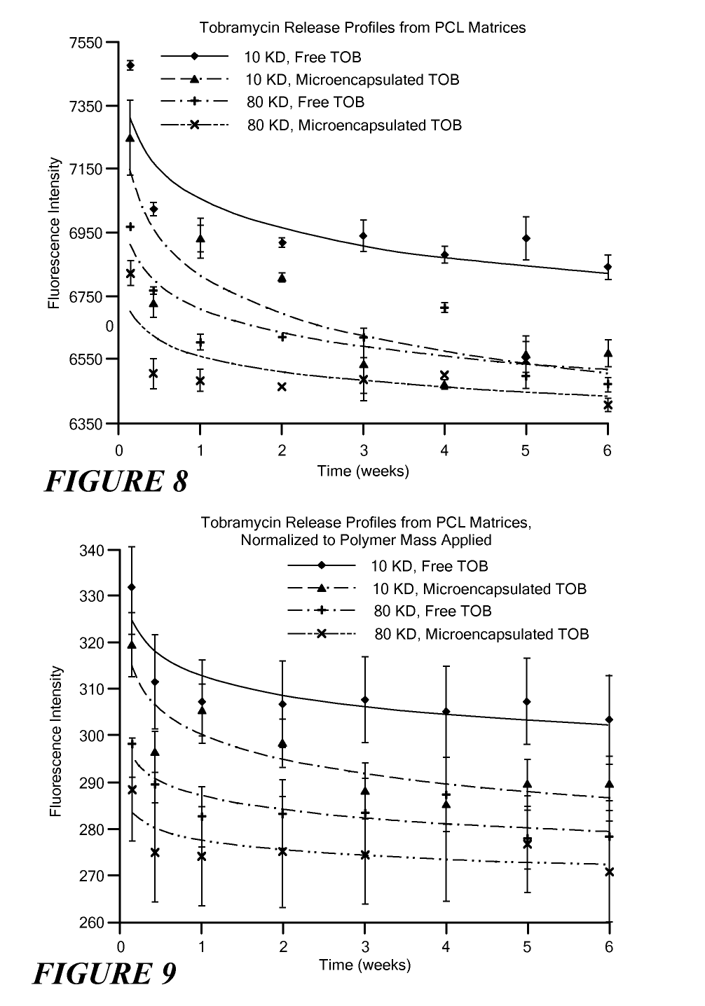

Cancellous bone chips treated with lower molecular weight PCL coatings and tobramycin release more drug at each timepoint. Microencapsulation of tobramycin reduces the amount of drug release at each time point (FIG. 8). Since drug loading is equal among each sample, release profiles are solely a function of the polymer matrix properties and micro-encapsulated drug loading. In both cases, the molecular weights (10,000 mol. Wt. and 80,000 mol. wt.) exhibit strong controlled release kinetics (FIG. 8) and reach near zero-order kinetics after a week. Furthermore, each absorbance is normalized to the amount of polymer (and likewise the amount of drug) that is loaded on each sample (FIG. 9). Given the variable nature of allograft bone as a substrate, in addition to the inability to apply extremely accurate amounts of material with the spray fabrication method at benchtop scale, applied polymer coating weight is normalized to minimize intra-cohort variability. The rates of drug release exhaustion from the polymers are quite similar, but the amount delivered per unit time is a function of the polymer molecular weight. Given the same drug loadings between a high and low molecular weight matrix, the low molecular weight PCL allows more rapid drug release and earlier dose exhaustion; the higher molecular weight PCL produces longer extended drug release.

3. A Method of Using Controlled Release Combination Biomaterials for Treatment of Osteonecrosis of the Femoral Head

a. Construction of Graft

Osteoconductive materials, such as allograft cancellous croutons, are cut to uniform size and weighed. A bioactive agent, such as gentamicin powder, is microencapsulated utilizing lipid spray microspheres. Six grams of PCL are dissolved in 150 milliliters of acetone at 47 degrees centigrade. Gentamicin powder and microencapsulated gentamicin are mixed with polycaprolactone (PCL) solution. About 6.25 mg of antibiotic is used for each bone specimen. A pressurized fine spray is used to apply the PCL solution directly to the cancellous bone pieces for uniform application. Air drying for 2 hours allows the acetone to dissipate, creating a thin coat on the bone. DBM is loaded with 40 mg/ml of gentamicin and packed into the PCL-coated crouton as shown in FIG. 1.

b. Implantation of the Graft

The dead bone is removed with a high speed burr 10 or other instruments. The viable bone chips are taken out from the femoral head and neck portion with a chisel or gouge for later use. If the femoral head is collapsed, the collapsed portion is elevated with an elevator or other instruments. A window made in the femoral head is trimmed for the insertion of the cancellous croutons. The size of the cancellous crouton should be matched to the window for tight impaction of the cancellous crouton. The cancellous crouton is then inserted into the femoral head, and bone chips and other biological materials are impacted into empty spaces between the cartilage cap and the cancellous crouton. The joint capsule is not closed. The donor site of the iliac crest is then reconstructed with the insertion of bone or with further polymer-coated bone graft substitute biomaterials. They are tied with a suture to the host bone in order not to be dislodged. The muscle fascia and subcutaneous tissue are repaired over a suction drain, and the skin is then closed.

c. Results

The biological agent is released from the cancellous crouton over a period of 6 weeks, or any therapeutically recommended time period. The patients is monitored postoperatively and shows no signs of infection over a period of 6 weeks, or as recommended by the surgeon.

4. A Method of Using Controlled Release Combination Biomaterials to Treat Injury to the Mandible

a. Construction of Graft

Osteoconductive materials, such as allograft cancellous croutons, are cut to uniform size and weighed. A bioactive agent, such as gentamicin powder, is microencapsulated utilizing lipid spray microspheres. Six grams of PCL are dissolved in 150 milliliters of acetone at 47 degrees centigrade. Gentamicin powder and microencapsulated gentamicin are mixed with polycaprolactone (PCL) solution. About 6.25 mg of antibiotic is used for each bone specimen. A pressurized fine spray is used to apply the PCL solution directly to the cancellous croutons or particles for uniform application. Air drying for 2 hours allows the acetone to dissipate, creating a thin coat on the bone. DBM is loaded with 40 mg/ml of gentamicin and packed into the PCL coated crouton as shown in FIG. 1.

b. Implantation of the Graft

The patient's injury site is prepped routinely as per surgery. The maxillo-mandibular occlusion is maintained by intermaxillary wire fixation to maintain jaw relations. The appropriate length of the transport segment should be estimated before surgery and a number of plates with different lengths should be available during surgery to choose from. The device can be fixed to the mandibular bone stumps either before or after removal of the tumor segment by three bicortical screws on each side as in traditional reconstruction plate, leaving out approximately 2 cm of bone at the edge of one of the two bone segments, classically the posterior segment, so that it can be separated and fixed to the transport unit. After tumor resection, the transport unit is fixed to the potential transport block (transport disc) through the two miniplates either before or after its separation.

The size of the cancellous crouton should be matched to the window for tight impaction of the cancellous crouton. The cancellous crouton is then inserted into the window, and polymer-coated drug releasing allograft graft particles, chips, autologous bone chips and other biological materials are impacted into empty spaces. The surgical site is secured and closed.

c. Results

The bioactive or pharmaceutical agent(s) will be released from the polymer-coated combination allograft biomaterial cancellous crouton over a period of 6 weeks, or any therapeutically recommended time period. The patients will be monitored postoperatively for signs of infection during hospital stay over a period of 6 weeks postoperatively, or as recommended by the surgeon.

5. A Robust Method to Coat Allograft Bone with a Drug-Releasing Polymer Shell

a. Sample Fabrication

Cancellous allograft bone croutons (Miami Tissue Bank) were massed and like size fragments were selected for each batch. Polycaprolactone (PCL, 10 kD) was dissolved at 100 mg/mL in acetone at 45° C. Tobramycin was added to the PCL solution as a 10% mass/volume ethanol solution. Each bone crouton was dip coated in the tobramycin/PCL solution and dried via methanol flocculation, vacuum drying, or air/heat drying. Each crouton was massed and the procedure was repeated to obtain a coating of approximately 20 mg of tobramycin containing PCL. Subsequently, a 10 kD PCL unloaded (no drug PCL solution) overcoat was applied in part of the cohorts (half of the croutons from each drying technique) and dried according to the same method as it was originally processed. Every crouton was massed again after application of the unloaded overcoat.

b. Methanol Flocculation

Methanol was filtered through the porous structure of some allograft cohorts (n=10) immediately following dip coating, precipitating the PCL from solution and removing excess, unbound PCL from the crouton.

c. Vacuum Drying

After dip coating, certain cohorts of croutons were placed in a vacuum flask and placed under vacuum pressure for approximately 3-5 minutes to quick dry the PCL acetone solution to the porous structure of the cancellous allograft crouton (n=12).

d. Air/Heat Drying

After dip coating, certain cohorts of croutons were allowed to dry on a sandbath for 15 minutes at 48° C., (n=10).

e. Scanning Electron Microscope (SEM) Imaging

Two croutons from each cohort (vacuum dried with or without an unloaded overcoat, methanol treated with or without an unloaded overcoat, air dried with or without an unloaded overcoat) were used for SEM (Hitachi S-3000N, Pleasanton, Calif.) imaging. Each sample was spattered coated with gold particles for approximately 4 to 7 minutes. Link Isis series 300 microanalysis system software displayed the real-time images captured by the microscope and allowed the capture of images varying between 1 mm to 500 micron magnification. Pore and fracture size was measured with PCI.

f. Tobramycin Release Kinetics

Three croutons from each cohort were individually submerged in 5 mL of phosphate buffered saline (PBS) and incubated at 37° C. PBS was collected at 6 time intervals: 30 minutes, 1 hour, 2 hours, 4 hours, 8 hours, and 24 hours. Tobramycin concentration was determined using a modified o-phthaldialdehyde (OPA)-based fluorescence assay (100 ul of sample, 100 ul of isopropanol, and 200 ul of OPA reagent (Sigma P-0532) incubated for 30 minutes at room temperature and read at excitation 360 nm and emission at 460 nm [7] using a microplate reader (Biotek spectrophotometer and GenePix5 software). A fresh sample of PBS was added at each time point.

g. Data Analysis

Concentrations and percent released were calculated based on a linear regression of tobramycin standards and the amount of tobramycin applied to each crouton system determined by the mass of the drug-PCL coating. Concentrations were plotted and one-way ANOVAs were used to determine statistical differences. Limits of detection for the assay were determined based on an extensive set of tobramycin standards and an optimized linear regression.

h. Results

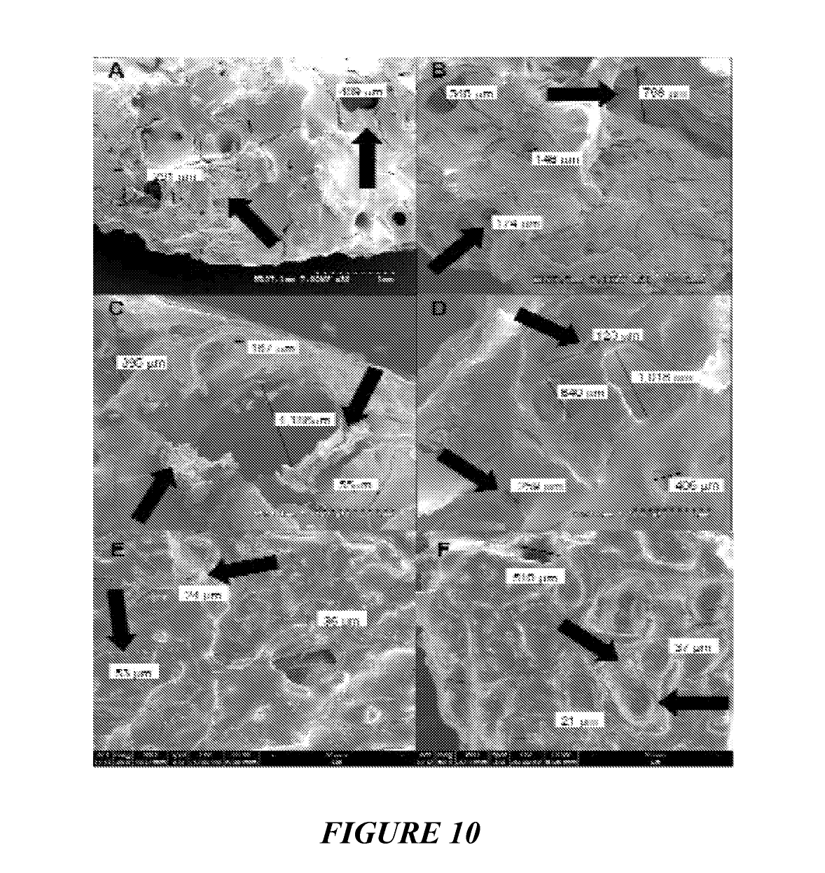

Macroscopically, all techniques provided approximately the same level of coating porosity; however, to determine coating integrity, scanning electron microscopy (SEM) was used. SEM imaging also showed no remarkable difference in observed pore sizes based on drying methods but revealed fractures in the coating emanating from the pores (FIG. 10). Compared to the other methods, air dried croutons displayed 1) a pore structure occluded with drug and polymer, 2) qualitatively less drug and polymer aggregated on the surface, and 3) limited surface fractures. Additionally, all images showed drug and polymer concentrated at the surface, with a greater amount shown on samples with an additional unloaded 10 kD PCL overcoat (FIGS. 10A and 10C).

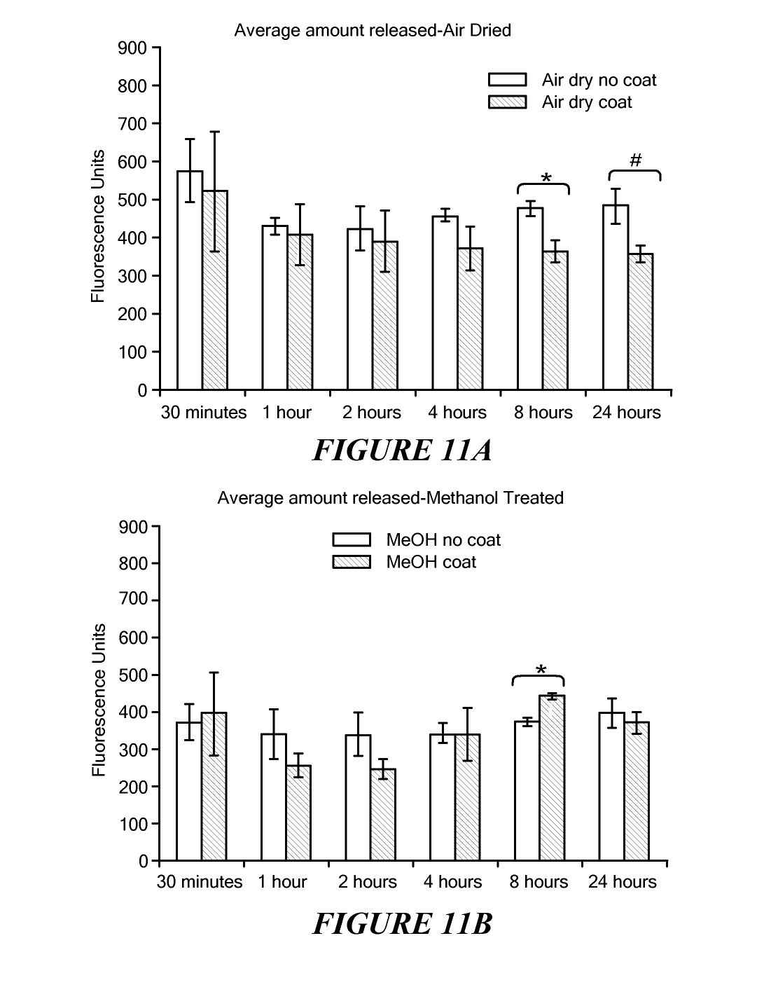

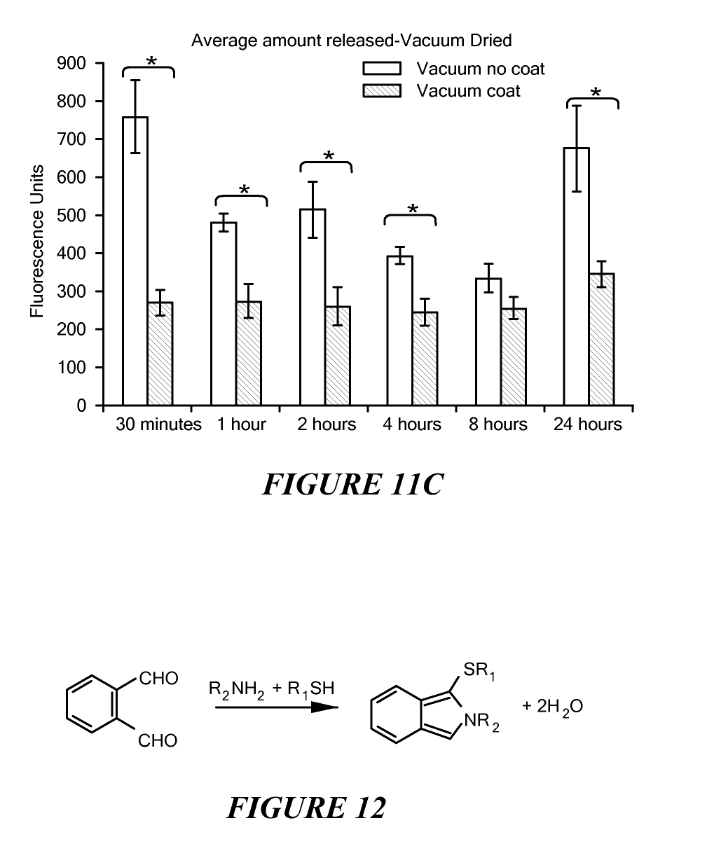

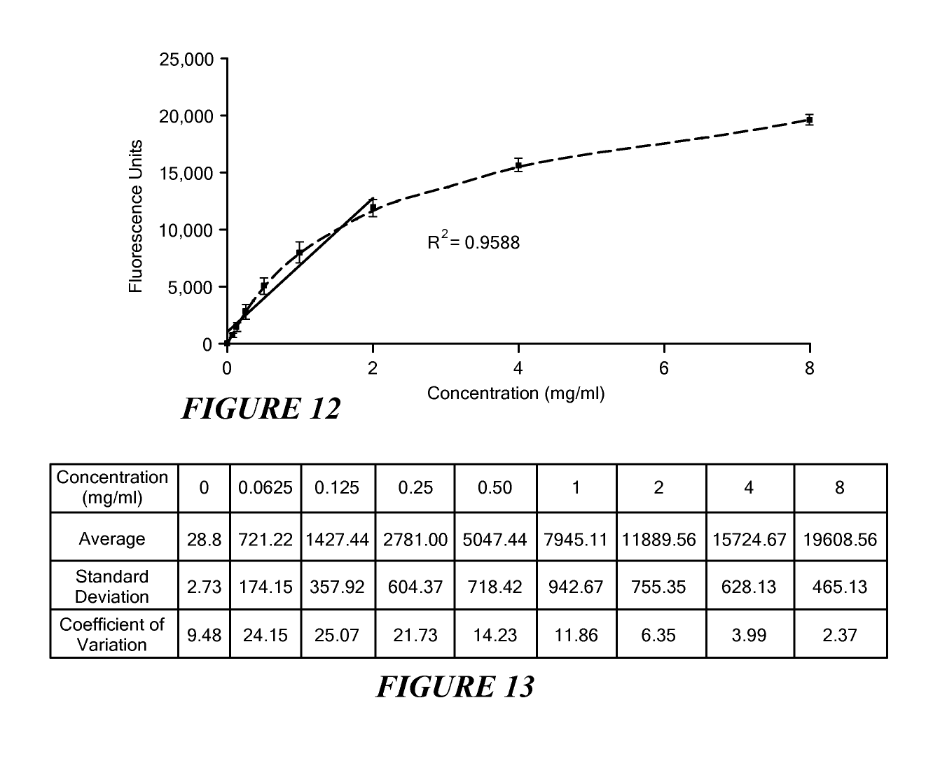

Because microscopic inconsistencies can impact the drug's burst release, short-term burst release kinetics were determined to assess the impact of drying and processing conditions (FIG. 11). Burst release, although often viewed as a hindrance for long-term controlled release systems, is necessary to combat wound-site pathogenic bacteria by rapidly achieving a local drug concentration above the minimal inhibitory concentration; however, burst release kinetics must also be controlled. In an attempt to modulate drug burst release, an additional 10 kD PCL unloaded overcoat was applied. OPA was reacted with tobramycin released from the croutons into PBS, and average fluorescence intensity was compared.

For the cohorts processed by air-drying and methanol flocculation, no significant difference between having or lacking the additional overcoat was observed (FIG. 11A, 11B) with the exception of methanol processed croutons at 8 hours (α=0.05, p<0.01) and air-dried at 8 hours (α=0.05, p<0.03) and 24 hours (α=0.1, p<0.07). Conversely, when croutons were vacuum-dried, addition of an unloaded overcoat greatly slowed the tobramycin release out to 24 hours (FIG. 11C). At 30 minutes there was a significant difference in a coated versus uncoated crouton (α=0.05, p<0.0007). Significant differences were also seen in the remaining time points, (α=0.1, 1 hr: p<0.002, 2 hr: p<0.02, 4 hr: p<0.006, [24 hr: p<0.02) with the exception of 8-hours. Alternately, drug burst release was modulated by the fragment processing conditions (i.e., air drying, vacuum drying or methanol flocculation). At 1 hour, 2 hours and 24 hours there was no significant difference between processing methods of croutons with an additional unloaded overcoat; however, there was a significant difference (α=0.1) between vacuum drying and air drying at 30 minutes (p<0.06), 4 hours (p<0.1) and 24 hours (p<0.06). At 8 hours there was an observed significant difference between methanol flocculation and air drying (α=0.1, p<0.06) as well as vacuum drying (α=0.1, p<0.003). For croutons without an overcoat there was no significant difference between any of the methods at 2 and 24 hours. There was a significant difference (α=0.05) between methanol flocculation and vacuum drying at 30 minutes (p<0.03) and 1 hour (p<0.04). At 4 (p<0.02) and 8 (p<0.01) hours, differences (α=0.05) were observed between methanol flocculation and air drying. Interestingly, vacuum and air drying only showed a significant difference at 8 hours (α=0.05, p<0.03).

6. Assay Method for Polymer-Controlled Antibiotic Release from

Allograft Bone to Target Orthopaedic Infections

a. Sample Fabrication

Cancellous allograft bone croutons (Miami Tissue Bank) were weighed and like-size fragments were selected for each cohort. PCL (Sigma CAS 24980-41-4, St. Louis, USA) (100 mg/ml) was dissolved in acetone at 45° C. Tobramycin (MP Biomedicals Cat #199696, Solon, USA) was suspended in the PCL acetone solution at 10% weight/volume. Cohorts were dip-coated in PCL/tobramycin solution (room temperature, 30 seconds to 1 minute). After vacuum drying (5-10 minutes), each crouton was weighed again to determine the amount of drug and polymer applied. Approximately 20 mg of polymer and 2 mg of tobramycin were applied per crouton.

b. Tobramycin Drug Relase

Coated croutons were individually submerged in phosphate buffered saline (PBS) and incubated at 37° C. PBS was collected at 24 hours, 72 hours, one, two, three, and four weeks.

c. HPLC Electrospray Mass Spectrometry

Tobramycin was analyzed using a YMC ODS-Aq 2.1×100 mm column with 5-micron particle size (Waters) on an HPLC coupled to positive ion electrospray (Agilent 1100 LC-MSD) mass spectrometer (mobile phase 80% A (0.2% PFPA in water)+20% B (acetonitrile) at 0.25 mL/min and 35° C. Benzoylecgonine-d3 was used as an internal standard (m/z 293).

d. Tobramycin Detection/OPA Assay

Stock OPA (o-phthalaldehyde) reagent was prepared as previously described. Briefly, 50 mg of OPA powder (Sigma P-0657) was dissolved in 4 ml of methanol, 0.5 ml of potassium borate (0.5 M boric acid adjusted to pH 10.4 with potassium hydroxide), and 50 ul 2-mercaptoethanol (Sigma M-6250) was added, and the solution was kept at 4° C. in the dark. Working reagent was prepared fresh each day by adding 50 ml of OPA stock solution to 1 ml of 0.5M potassium borate buffer. Each detection reaction included 100 ul of release sample in PBS, 100 ul of isopropanol (Mallinckrodt Baker, Phillipsburg, USA #3032-22), and 200 ul of OPA reagent (Sigma P-0657). Each reaction was assembled in a 1.5 ml microcentrifuge tube, vortexed, and incubated at room temperature in the dark for 30 minutes. Tobramycin standards as internal controls were made in PBS and placed in each 96-well UV black-wall assay plate (Costar #3631) with samples. Fluorescence for each derivatization reaction (300 ul) was detected (excitation 360 nm and emission at 460 nm) in a Biotek spectrophotometer and GenePix5 software (BioTek, Winooski, USA).

e. Data Analysis

Fluorescence readings for the standard curves were fit with linear regression and used to calculate concentrations of tobramycin released in each sample. Amounts of tobramycin were calculated based on the weight added to each crouton and percent of tobramycin added to the coating formulation. Percent drug release was calculated by dividing the amount of tobramycin released by the amount of tobramycin in each crouton multiplied by 100. Pairwise one-way ANOVAs were used to identify significant differences.

f. Results

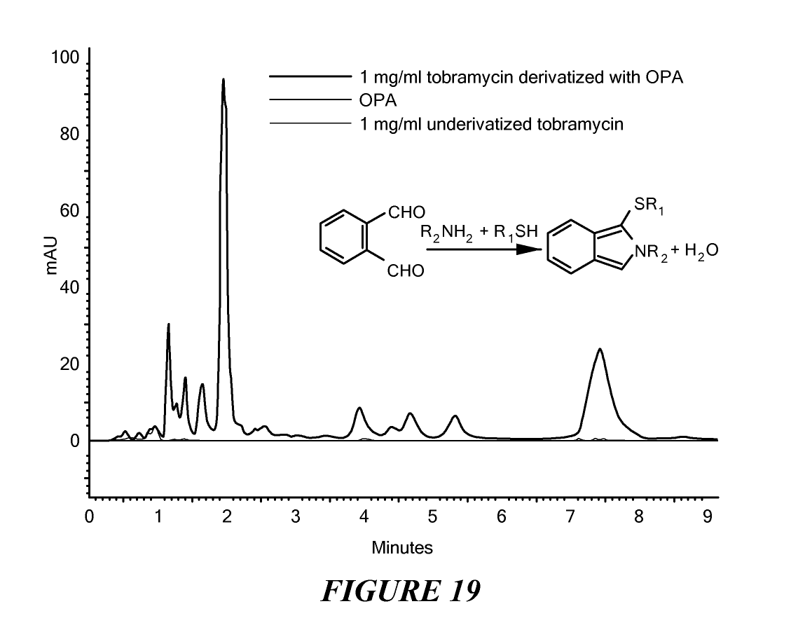

Because facile detection of tobramycin is often complicated by the derivatization protocol used, OPA provided a sensitive reliable reagent to derivatize the primary amines of tobramycin (FIG. 19). Reliability of the 96-well assay format was assessed by averaging fluorescence signal from known tobramycin concentrations over multiple runs, and determining the error for each standard (FIG. 13). OPA was reacted with the 8 mg/ml tobramycin standard in isopropanol for 30 minutes at which time the reaction was serially diluted in PBS to provide 8 standards from 8 mg/ml to 0 (blank). Standards from 9 assays performed on different days were used for all calculations. Standard errors ranged between 314 and 1 fluorescence units (FU) with the largest errors seen at the upper limits of the linear range (as determined by the high R value) of the assay at 2 mg/ml (252 FU), 1 mg/ml (314 FU), and 0.5 mg/ml (239 FU). PCL does elicit a fluorescence response (˜500 FU), thus the lower detection limit of the assay was limited to approximately 125 μg/ml.

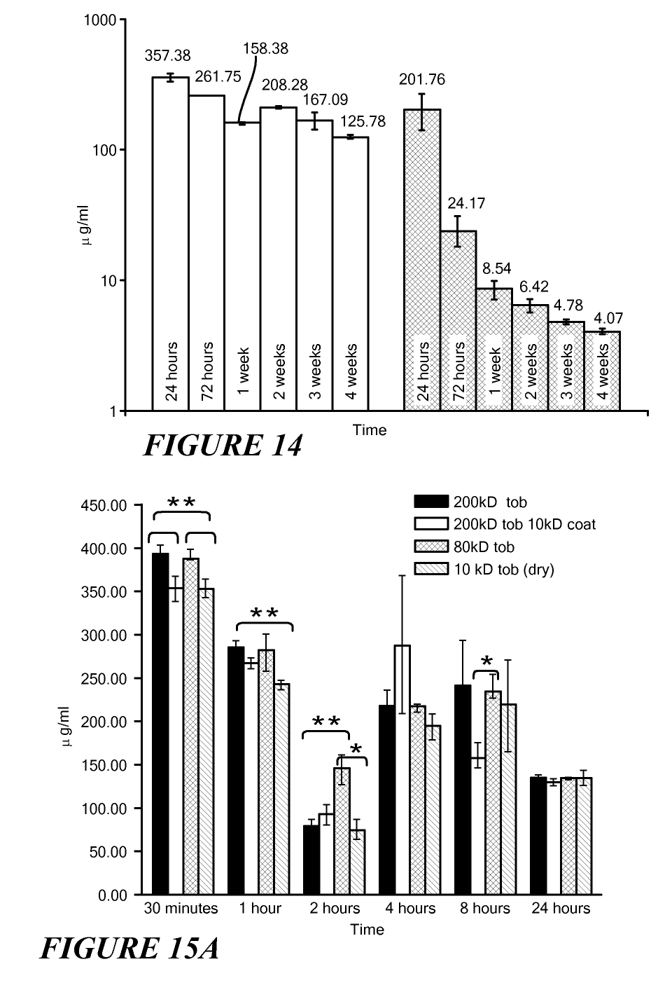

To validate the newly developed OPA-based tobramycin assay, two samples from one cohort (200 kD PCL/drug with an unloaded 10 kD PCL overcoat) were compared using the OPA assay and mass spectrometry methods (FIG. 14). Tobramycin concentrations measured based on the OPA assay were calculated using the tobramycin standard curves (all regressions had R² values>0.92). Drug amounts measured in PBS via mass spectrometry were significantly lower than amounts determined by the OPA assay with the exception of the 24-hour time point in which there was no significant difference (α=0.05).

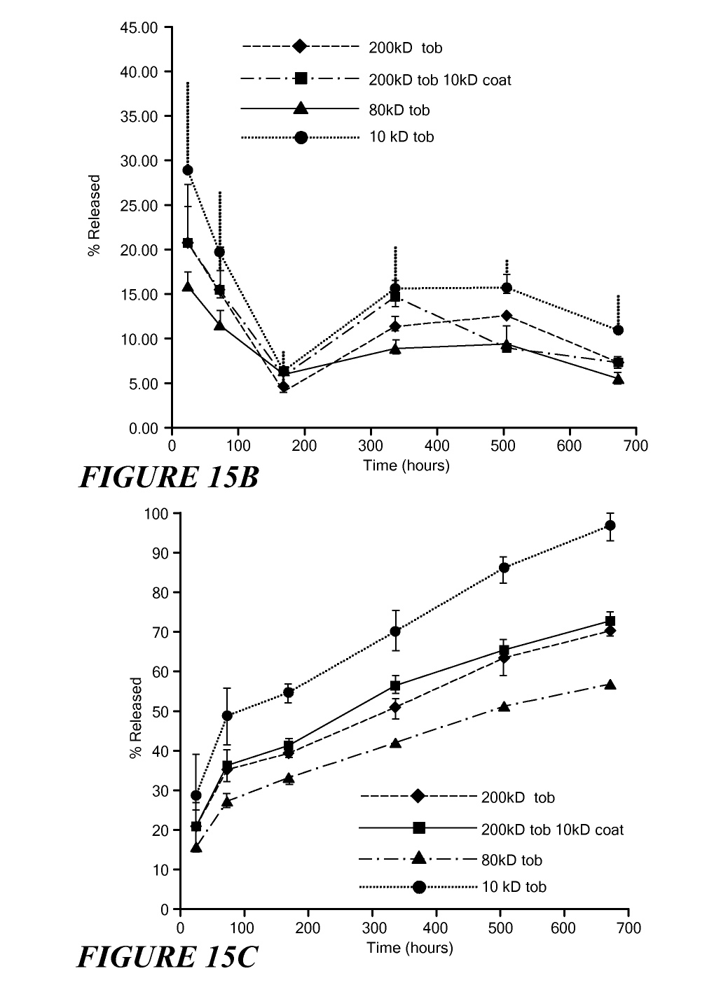

To determine the utility of the OPA fluorescence assay for the PCL-drug loaded coating release, cohorts of PCL-controlled, tobramycin-loaded coated allograft fragments were assayed over time and drug release kinetics were calculated. Solutions of varying molecular weight PCL (10 kD, 80 kD, and 200 kD) and 10% w/v drug concentration were used to coat cancellous allograft fragments in different cohorts. An additional cohort used a final 10 kD PCL blank coat without tobramycin as an “unloaded overcoat” over a 200 kD tobramycin-containing coating. FIG. 15 shows drug release kinetics determined via the 96-well OPA fluorescence assay, demonstrating the ability of the assay to illuminate differences in release kinetics from varying PCL coating molecular weight. In the first 24 hours, each cohort, exhibited a burst release with the largest release emanating from the smallest molecular weight PCL coating (FIGS. 15 A and 15B). However, there were significant differences (10 kD and 200 kD (α=0.1, p<0.05), 10 kD and 80 kD (α=0.1, p<0.08), 200 kD and 200 kD with a 10 kD unloaded overcoat (α=0.1, p<0.09)) in the amount of tobramycin bursting from each coating. After the initial bolus, amounts of tobramycin released decreased to a minimal amount at week 1 (significant differences between 200 kD and 80 kD (α=0.1, p<0.025), 80 kD and 10 kD (α=0.05, p<0.025)) rebounded to approximately half of its 24 hour levels and leveled off through week 4. There was still a significant difference between the 80 kD coating and the 200 kD coating with an unloaded 10 kD overcoat at 3 weeks (α=0.05, p<0.05), but all coatings released approximately the same amount of tobramycin at 4 weeks. By 5 weeks, the amount released was negligible. When coated with 10 kD PCL, 100% of the tobramycin payload was released within 4 weeks (FIG. 15C). At each time point, with the exception of one week for certain cohorts, the amount of tobramycin released was above the measured minimal inhibitory concentration against E. coli. (˜1 ug/ml) (FIG. 15A).

7. Evaluating Antibiotic Release Profiles as a Function of Polymer Coating Formulation

a. Sample Fabrication

Cancellous allograft bone croutons (Miami Tissue Bank) were weighed and similar weights were selected for each cohort. 10 kD or 80 kD PCL (Sigma CAS 24980-41-4, St. Louis, USA) (100 mg/ml) was dissolved at 45° C. in acetone with 4% v/v deionized water. Tobramycin (MP Biomedicals Cat. #199696, Solon, USA) was suspended in the PCL/acetone/water solution at 10% weight/volume. Cohorts were dip-coated in PCL/tobramycin solution (room temperature, 30 seconds to 1 minute). After incubating for 5 minutes at −20° C., croutons were vacuum dried (5-10 minutes). Croutons were dipped 4-6 times. Each crouton was weighed after coating to determine the amount of drug and polymer applied to each crouton. For all comparisons, the amount of drug released was normalized to the amount of drug applied to the crouton based on the weight of applied coating as well as the percent of tobramycin in the formulation.

b. Tobramycin Drug Release

Coated croutons were individually submerged in 3 ml of phosphate buffered saline (PBS, cat#BP661-10, Fisher Scientific) and incubated at 37° C. The complete release volume was collected and replaced at 24 hours, 72 hours, and each week up to six weeks. A 96-well colorimetric assay was used to compare the release kinetics for each formulation as previously reported.

c. High Performance Liquid Chromatography (HPLC)

Samples were analyzed using HPLC as previously described. Briefly, tobramycin was derivatized using OPA reagent. Each sample (10 ul) was injected 30 minutes after addition of the OPA derivatizing reagent using a 1 ml/min flow rate and data was analyzed using both a fluorescence detector (Ex₃₄₀, Em₄₅₀) and a UV-Vis (254 nm) detector. The area under a specific tobramycin peak was plotted against standard concentration and the data were fit to a linear regression as a standard curve. Concentration of unknown release samples were calculated from the standard curve using regression analysis.

d. Microbiology

Release samples (500 ul) were concentrated in a heated-vacuum centrifuge (Labconoco Centrivap, Kansas City, Mo.) and prepared in low-bind, non-tissue culture treated 96-well microtiter plates (MIC—round bottom, ZOI—flat bottom). All samples were then stored dry at 4° C. until use.

e. Bacteriostatic Assay

LB broth (100 ul, cat#244620, Difco) was added to each well of the round bottom 96-well plate to reconstitute the lyophilized drug release samples. Each well was inoculated with 10⁵ CFU (10⁵=OD₆₀₀˜1) of a liquid culture of E. coli (ATCC 25922). The plates were incubated overnight at 37° C. Plates were imaged using UV (Bio-Rad, Hercules, Calif.) and growth was visually determined via comparison with known standard tobramycin concentrations.

f. Zone of Inhibition (ZOI)

For ZOI experiments, release samples were dried onto 6 mm Whatman 1 filter paper disks. Muller Hinton agar plates (cat#B21800X, Fisher Scientific) were prepared by streaking E. coli (ATCC 25922) (10⁵ CFU) to create a contiguous lawn of bacterial growth. Disks containing the dried-down drug were then placed with a minimum distance of 24 mm between each disk and the side of the plate. Plates were incubated overnight at 37° C. The diameter of the zone of inhibition or bacterial clearing around each disk was measured.

g. Data Analysis

The amount of tobramycin released in each sample was calculated based on the linear regression of the fluorescent units (FU) for each standard. Percent drug release was calculated by dividing the amount of tobramycin released by the amount of tobramycin in each formulation and multiplying by 100. All formulations were tested in triplicate (biological and technical replicates) and Excel was used to calculate the average and standard deviation. Pairwise one-way ANOVAs were used to identify significant differences (p<0.05).

h. Results

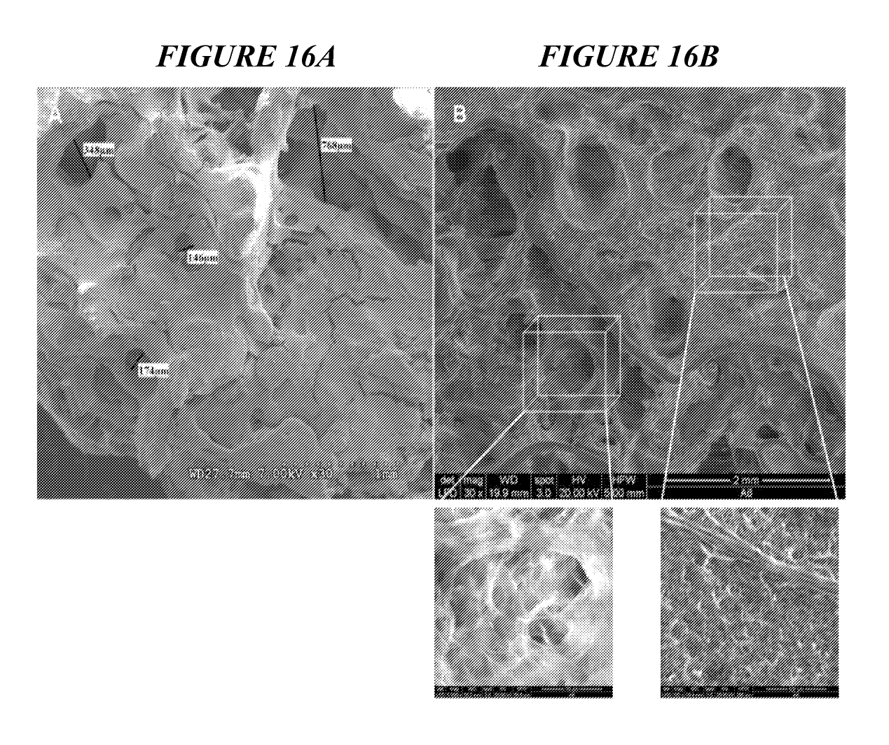

Variations in coating formulations of allograft bone yielded differences in physical characteristics of the surfaces. FIG. 16 portrays visual differences between two different coating techniques analyzed by SEM imaging. Differences can be explained by either variations in the coating formulation or differences in the application technique used. Coating inconsistency was evident for formulations in which the polymer and antibiotic were dissolved in acetone prior to dip-coating application. Additionally, cracking and occlusion of the porous cancellous structure was also apparent following air drying (FIG. 16A). Alternatively, when 4% non-solvent water was added to the formulation in addition to a freeze drying step, little or no cracking was observed (FIG. 16B). The freeze-drying procedure produced an intricate lattice structure in addition to the overall structural porosity of the cancellous allograft fragments (FIG. 16B insets).

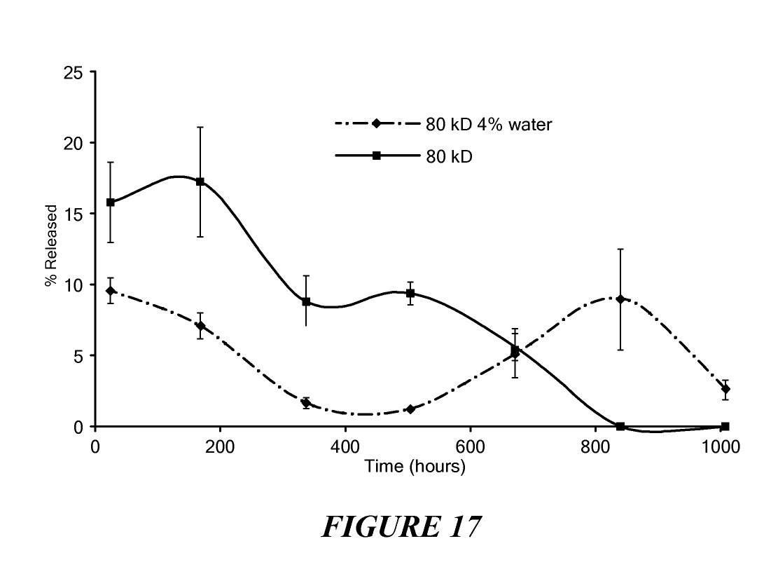

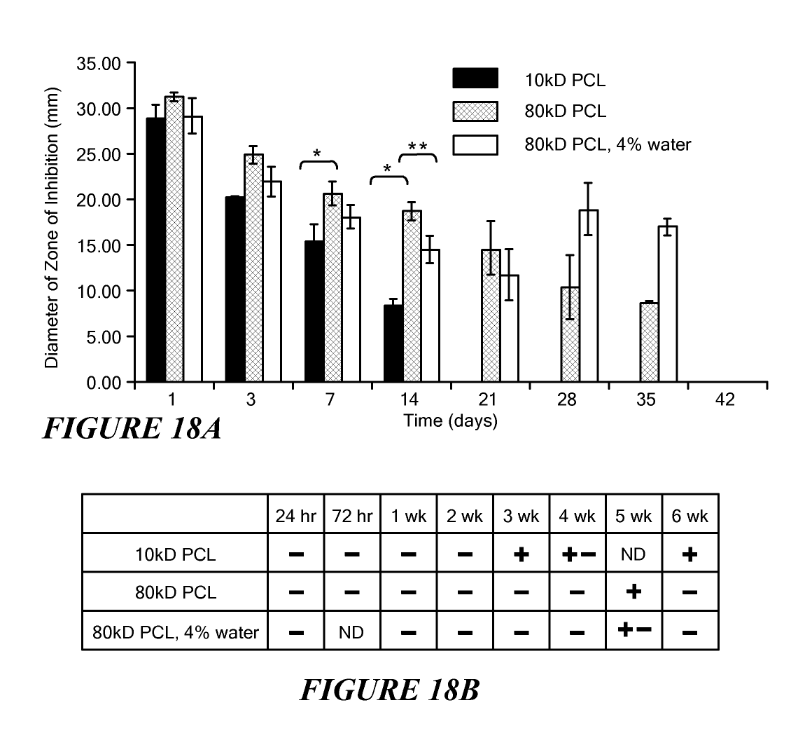

Tobramycin was derivatized via a chemical reaction with OPA and detected using product fluorescence. HPLC confirmed that tobramycin elicited no fluorescent or absorbance in the absence of OPA (FIG. 19); additionally, OPA exhibited very limited background fluorescence (data not shown). Thus, the fluorescence of OPA-derivatized tobramycin was detected via a 96-well assay. Polymer coating formulations were compared using this assay to reveal differences in release kinetics (FIG. 17). Drug anti-microbial activity was assessed using both bacteriostatic and zone of inhibition studies. Bacterial killing varied among three coating techniques, although points of significance were minimal (FIG. 18).

8. Polymer-Controlled Release of Tobramycin from Allograft Bone Void Filler

a. Fabrication of Polymer-Coated Allograft Fragments

Cancellous allograft bone fragments (Miami Tissue Bank) were weighed and like-size fragments were selected for each cohort. Alternatively, micron-size allograft bone particulate matter (Miami Tissue Bank) was partitioned into 100 mg aliquots for coating. PCL (Sigma CAS 24980-41-4, St. Louis, USA) (100 mg/ml or 60 mg/ml) was dissolved in acetone at 45° C. Unencapsulated tobramycin (MP Biomedicals Cat #199696, Solon, USA) was suspended in the PCL acetone solution at 10-30% weight/volume depending on the formulation. Certain coating formulations included tobramycin commercially encapsulated in vegetable oil (microencapsulated) (70% tobramycin, Lot#TM 150-70-30, Maxx Performance Inc., Chester, N.Y.). Formulations and cohorts are detailed in Table 1. Dip-coated cohorts were prepared by placing allograft bone into the PCL/unencapsulated tobramycin solution at room temperature. Fragments were removed after soaking in polymer solution for 30-60 seconds. After vacuum drying (5-10 minutes at ambient temperature), each fragment was weighed again to determine the amount of drug and polymer applied. Particulate cohorts were coated with 1 ml of solution that was subsequently allowed to flash off, leaving coated particulate. To alter drug release kinetics, 35-45% PEG (polyethylene glycol) and/or microencapsulated tobramycin were either mixed with the PCL solution or coated in alternating layers with it. Cohorts were dip-coated as described herein.

TABLE 1

| | | | | | | Allograft |
|---|---|---|---|---|---|---|
| Cohort | PCL | | PEG | Tobramycin | Type of coating | Bone |
| 1 | 100 | mg/ml | 0 | Unen- | Dip-coat | Partic- |
| | | | | capsulated | | ulate |
| 2 | 100 | mg/ml | 35% | Unen- | Dip-coat | Partic- |
| | | | | capsulated | | ulate |
| 3 | 100 | mg/ml | 0 | Unen- | Dip-coat | Fragment |
| | | | | capsulated | | |
| 4 | 60 | mg/ml | 0 | Unen- | Dip-coat | Fragment |
| | | | | capsulated | | |
| 5 | 60 | mg/ml | 45% | En- | Layer-by-Layer | Partic- |
| | | | | capsulated | (PCL/PEG/PCL) | ulate |
| 6 | 60 | mg/ml | 45% | En- | Layer-by-Layer | Fragment |
| | | | | capsulated | (PCL/PEG/PCL) | |

b. Drug Release

Each sample was released into 3 ml of phosphate buffered saline pH 7.4 (PBS, cat#BP661-10, Fisher Scientific). In certain experiments, the complete release volume was drawn off and replaced at 30 minutes, 1 hour, 2 hours, 4 hours, 8 hours, 24 hours, and each week for up to 6 weeks to simulate sink conditions. Kinetics of release from each formulation were compared via a 96-well colorimetric assay previously reported. Amounts of tobramycin released were normalized to the theoretical amounts applied and are reported as a percent to facilitate comparison of different polymer formulations.

c. High Performance Liquid Chromatography (HPLC)

Samples were analyzed in triplicate using high pressure liquid chromatography (HPLC). Prior to HPLC, tobramycin was derivatized with OPA as previously described. At the completion of the reaction (30 minutes), 200 ul of the derivatization reaction was transferred to a plastic HPLC vial. Data was collected from both a fluorescence detector (ex=350 nm, em=450 nm) as well as UV-Vis detector (340 nm). Samples were analyzed using a Hypersil GOLD HPLC column (Thermo Fisher Scientific, 100*4.6 mm LOT #10377) and ChromQuest 5.0 (Thermo Scientific) software on a Finnigan Surveyor (Thermo Scientific) system. Each sample (10 ul) was injected using a 2 ml/min flow rate. The mobile phase was 0.02M phosphate pH 6.5:acetonitrile (52:48). The area under the tobramycin peak was plotted against standard concentration and the data were fit to a linear regression as a standard curve. The regression equation was used to calculate the concentration of unknown release samples.

d. Spectral Shift Assay

Tobramycin (1 mg/ml) was analyzed in the presence or absence of the OPA reagent to validate the derivatization reaction. The OPA reagent in the absence of tobramycin was included as a control. Tobramycin was derivatized as previously described and assessed using the HPLC methods above.

e. Microbiology

Release samples (500 ul per experiment) for all microbiology studies were concentrated in a heated-vacuum centrifuge (Labconoco Centrivap, Kansas City, Mo.) and prepared in low-bind, non-tissue culture treated 96-well microtiter plates according to their subsequent experimental use (MIC—round bottom, ZOI—flat bottom). All samples were stored dry at 4° C. until use. Bioactivity after concentration as well as storage was confirmed with control conditions.

f. Bacteriostatic Assay

LB broth (100 μl, cat#244620, Difco) was added to each well of the round bottom 96-well plate to reconstitute the lyophilized drug release samples. Each well was inoculated with 10⁵ CFU (10⁵=OD₆₀₀˜1) of a liquid culture of E. coli (ATCC 25922). Liquid bacterial cultures were prepared using a sterile swab to select 1-3 isolated colonies from a blood agar plate (Remel cat#R01200). The plates were incubated overnight at 37° C. Bioactivity, as assessed by growth inhibition, of the released drug was visually determined via comparison with known standard tobramycin concentrations. Growth inhibition was designated if the visual turbidity of bacterial growth differed from the positive control by 80%. Negative growth was determined when the well was free of bacterial debris.

g. Zone of Inhibition (ZOI)

For ZOI experiments, release samples were dried onto 6 mm Whatman 1 filter paper disks. Muller Hinton agar plates (cat#B21800X, Fisher Scientific) were prepared by streaking E. coli (ATCC 25922) (10⁵ CFU) to create a contiguous lawn of bacterial growth (turbidity adjusted to a 0.5 McFarland standard using a Nephelometer (Phoenix Spec, BD Falcon)). Disks containing the dried-down drug were then placed with a minimum distance of 24 mm between each disk and the side of the plate. Plates were incubated overnight at 37° C. Calipers were used to measure the diameter of the zone of inhibition or bacterial clearing around each disk.

h. Data Analysis

The amount of tobramycin released in each sample was calculated based on the linear regression of the fluorescent units (FU) for each standard. Amounts of tobramycin were calculated based on the weight added to each allograft bone sample and percent of tobramycin added to the coating formulation. Percent drug release was calculated by dividing the amount of tobramycin released by the amount of tobramycin in each formulation multiplied by 100. All formulations were tested in triplicate (biological and technical replicates) and Excel was used to calculate the average and standard deviation. Pairwise oneway ANOVAs were used to identify significant differences (p<0.05 for significance).

i. Results

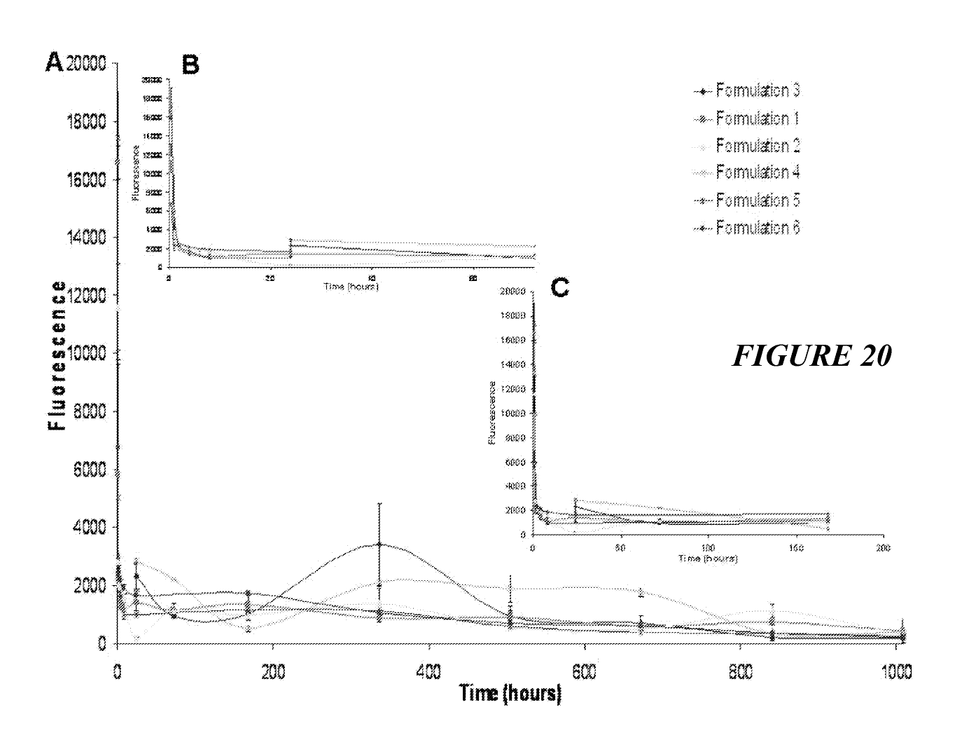

Samples were fabricated according to Table 1. Amounts of tobramycin applied to each sample were calculated based on the weight of coating applied to the allograft bone material and the amount of tobramycin included in the formulation. Tobramycin was derivatized with o-pthaldehyde (OPA). The derivatization reaction was verified via HPLC. In the absence of OPA, tobramycin did not elicit any absorbance (FIG. 19) or fluorescence response. Similarly, OPA did not elicit a background absorbance in acetonitrile or in PBS in the absence of tobramcyin. HPLC peaks were only seen when tobramycin was reacted with OPA. Tobramycin was released from the polymer into PBS. PBS was siphoned at designated time points and replaced to simulate sink conditions. Tobramycin content at each time point was assessed via a 96-well colorimetric assay (FIG. 20). Different formulations were not significantly different although formulation 4 (Table 1) did consistently yield a higher amount of tobramycin.

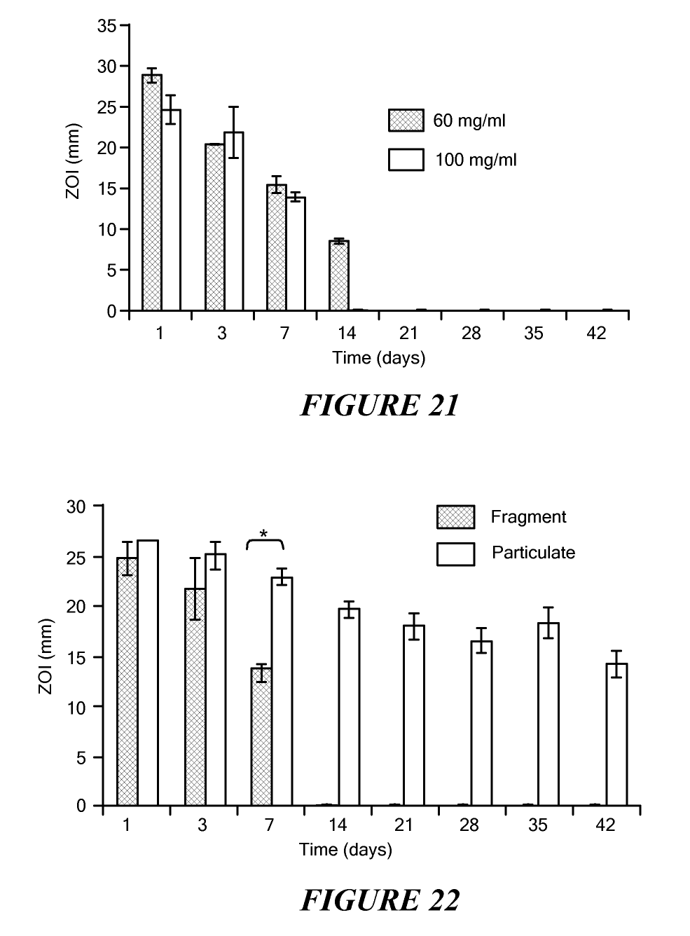

Based on the derivatization reaction, the efficacy of polymer formulations was compared using a 96-well colorimetric assay to detect tobramycin. Coating formulations were changed in an effort to alter the tobramycin release kinetics, specifically the kinetics of burst release within the first 24 hours. Therefore, the concentration and ratio of PEG and PCL in a coating formulation were changed and the drug release kinetic curves were compared (FIG. 21). Certain formulations (i.e., 60 mg/ml PCL, PCL/PEG mixture, 100 mg/ml particulate) released tobramycin out to six weeks while other formulations (i.e., PCL/PEG layers, 100 mg/ml PCL coated fragments) fell short of this therapeutic window.

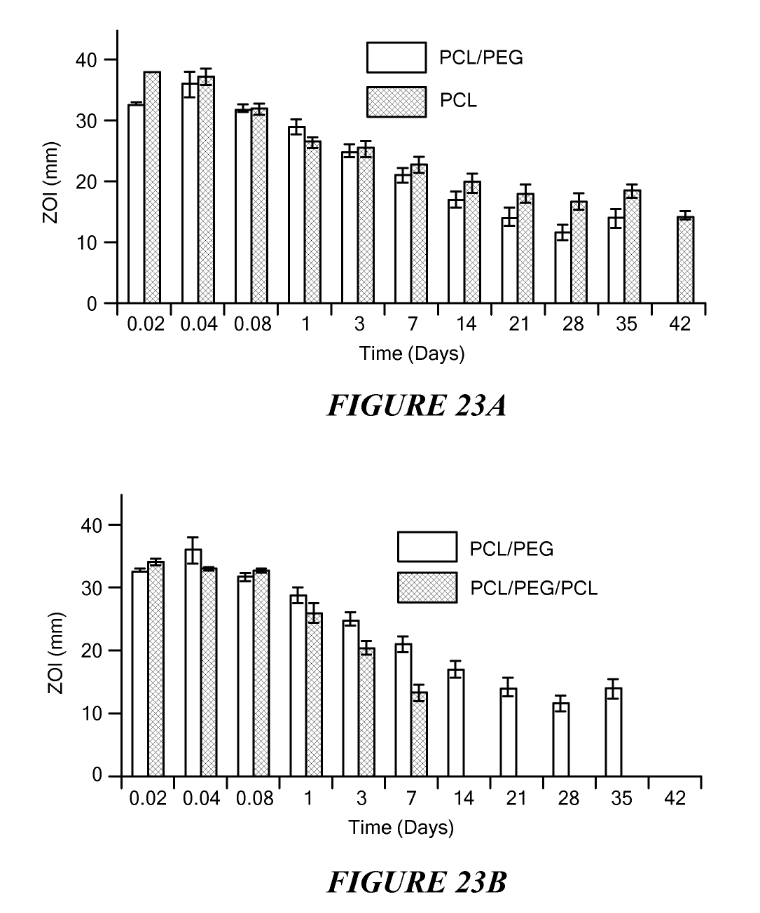

Regardless of the formulation, bioactivity was confirmed via an in vitro bacteriostatic assay designed based on a modification of the standard techniques for determining the minimal inhibitory concentration (MIC) for an antibiotic (data not shown) as well as a zone of inhibition or a radial diffusion assay (FIGS. 21, 22, 23). Tobramycin release was affected not only by the PCL concentration (FIG. 21) but also the form of the allograft material (fragments or micron-sized particulate) (FIG. 22) as well as the PEG addition (FIG. 23 A) and application method (FIG. 23B). Longevity of release was increased inversely to PCL concentration. Release was extended by one week when PCL content was decreased by 40%. Tobramycin release from micron-sized allograft material was significantly higher (p=0.038) at seven days compared to allograft fragments of similar weight as well as having an extended duration of bioactivity. Additionally, mixing 45% PEG into the PCL formulation decreased the effective release by one week; however, with no significant difference in the size of the zone of inhibition at all the preceding time points. There was, however, a decrease in longevity observed when alternating layers of PCL and PEG (PCL/PEG/PCL). PCL was applied at a concentration of 60 mg/ml while PEG was at a 45% (w/v). Each layer was allowed to dry completely (as indicated by weight) prior to the subsequent layer being applied. Tobramycin released into PBS from coated micron-sized particulate provided an effective zone of inhibition out to 42 days while tobramycin released into PBS from allograft bone fragments only provided bioactive tobramycin out to 7 days. This difference reflects not only differences in the surface area of the allograft bone, although approximately the same amount of allograft bone (approximately 100 mg) was coated, but also in the coating application as well. Additionally, antibiotic-containing polymer not coating micron-sized allograft bone particulate can also be included.

9. In Vivo Procedures

Mice were anesthetized by intraperitoneal injection of ketamine/xylazine (ketamine: 75 mg/kg, xylazine: 25 mg/kg). A small incision was made on the back of the anesthetized mouse perpendicular to the vertebral column and above the scapula. Implant sites were created using blunt scissors to tunnel just beneath the cutaneous trunci parallel to the vertebral column to open a small (˜1 cm) subdermal pouch into which the implant and innoculum were placed.

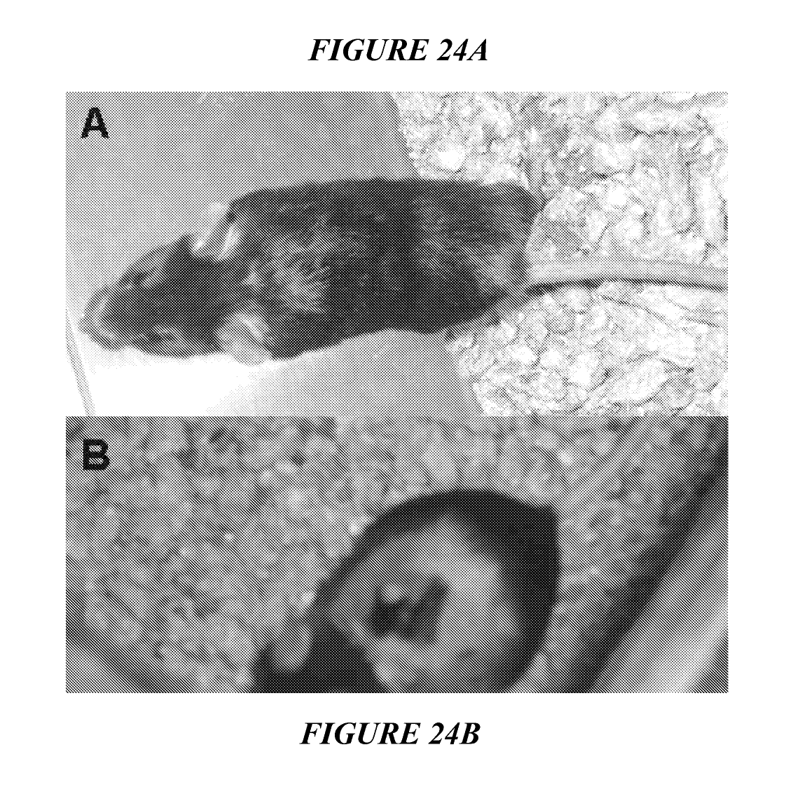

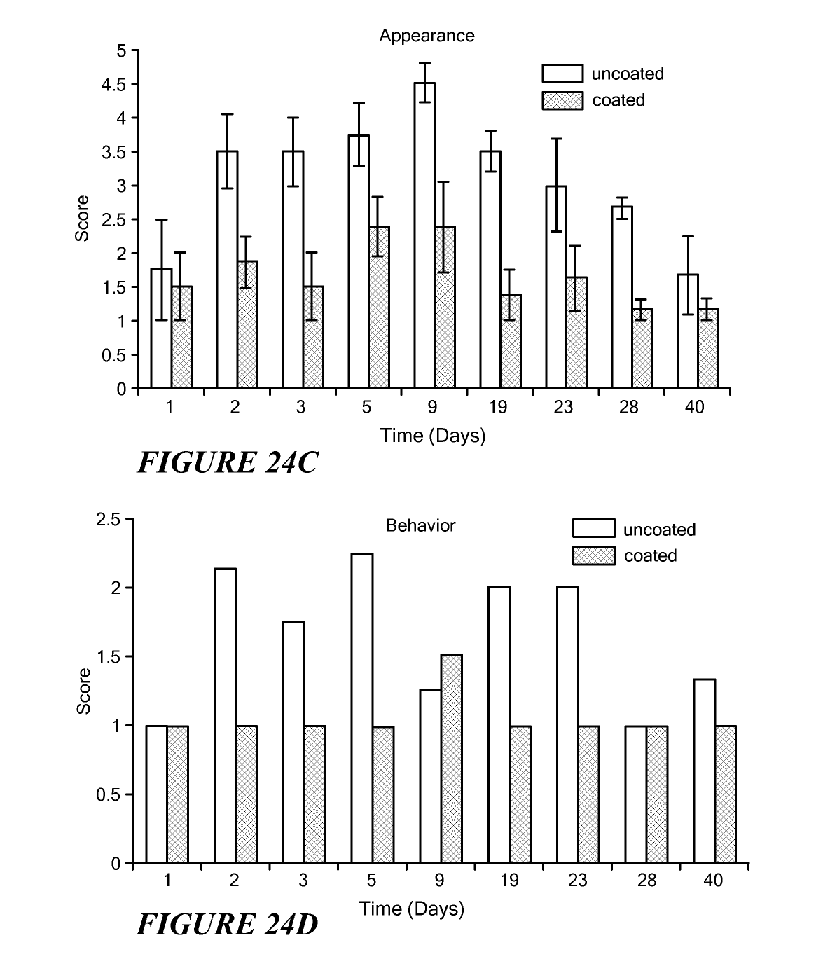

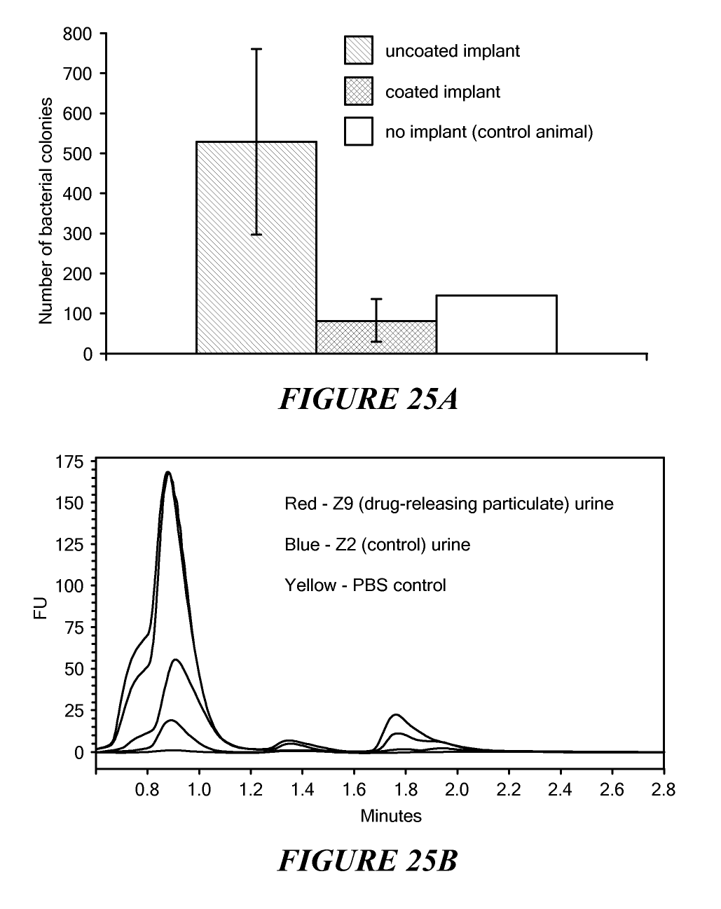

Approximately 100 mg of allograft particulate bone was placed in the pouch. Control animals were implanted with uncoated allograft particulate (FIG. 24A) while test animals (FIG. 24B) were implanted with polymer-controlled antibiotic-releasing coated allograft particulate. The small incision was sutured and closed with tissue adhesive. After the surgical site was closed, approximately 10⁵ CFU of E. coli (ATCC 25922) resuspended in pre-warmed saline was injected (<0.1 ml volume) into the subdermal pouch, located by manual palpation. Animals were visually monitored for pain or stress and scored for appearance and behavior (FIG. 24). Appearance was assessed based on a visual inspection of the animal's fur, and the condition of the surgical site (i.e. swollen, red, exudite) and assigned a value of 1-5 with 5 indicating the most signs of infection and poor healing (FIG. 24C). Alternatively, behavior and movement was also assessed as an indication of distress and infection and assigned a value of 1-5 with 5 indicating the most distress (FIG. 24D). Animals implanted with coated particulate had an average better appearance than those implanted with uncoated particulate. Behavior did not seem to be as affected by the presence of the antibiotic-containing polymer. Blood and urine were also assessed with HPLC 24 hours post surgery (FIG. 25B). Urine was also evaluated for the presence of bacteria (FIG. 25B). Animals implanted with the antibiotic-containing implant had a reduced bacterial load in the urine when compared to the control animals implanted with uncoated allograft particulate and no implant control animals. To reduce pain after the surgical procedure, buprenorphine was administered as an analgesic for 2 days post surgery.

10. Antibiotic Release from Allograft Bone Vs. Proosteon 500R™ Synthetic Graft

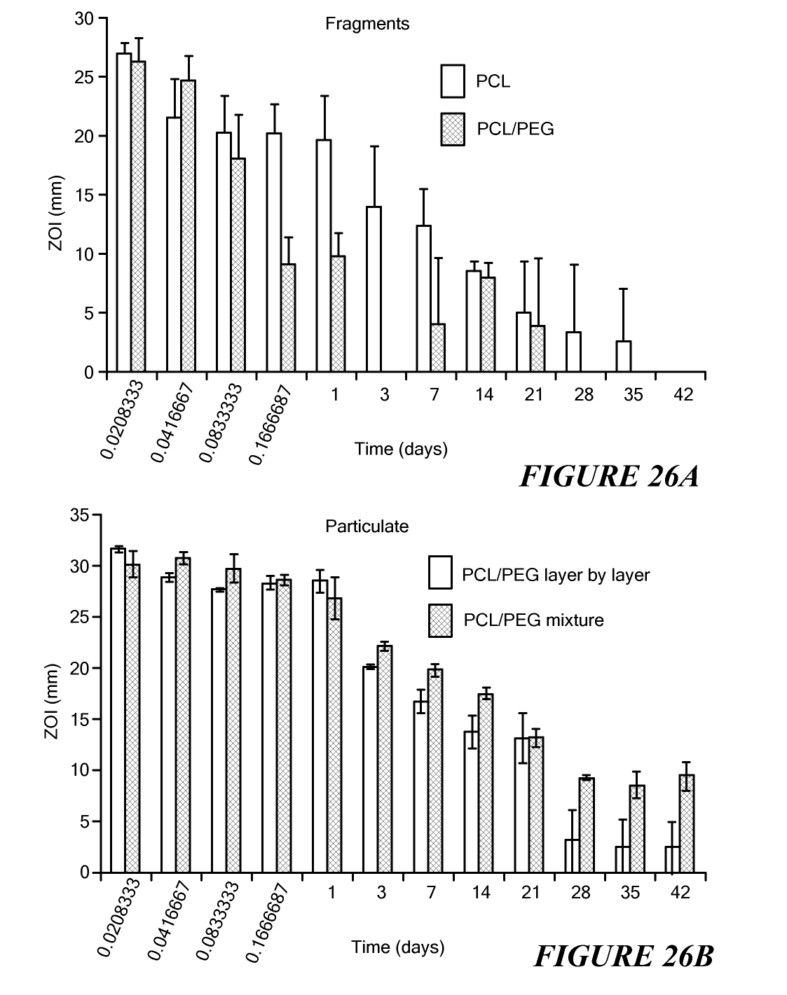

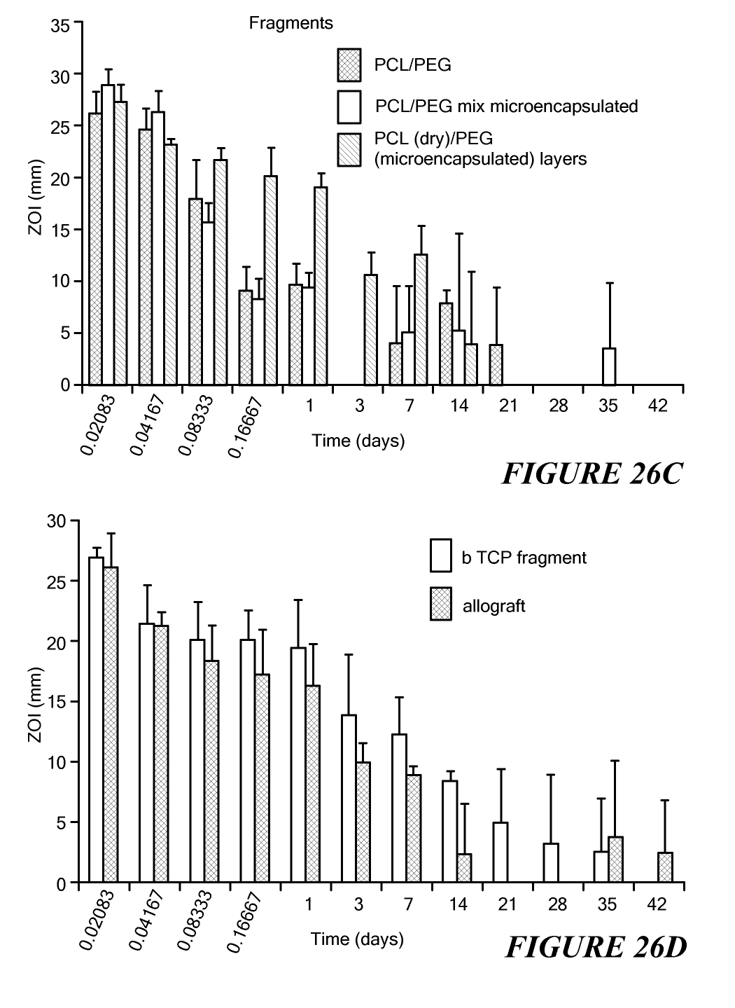

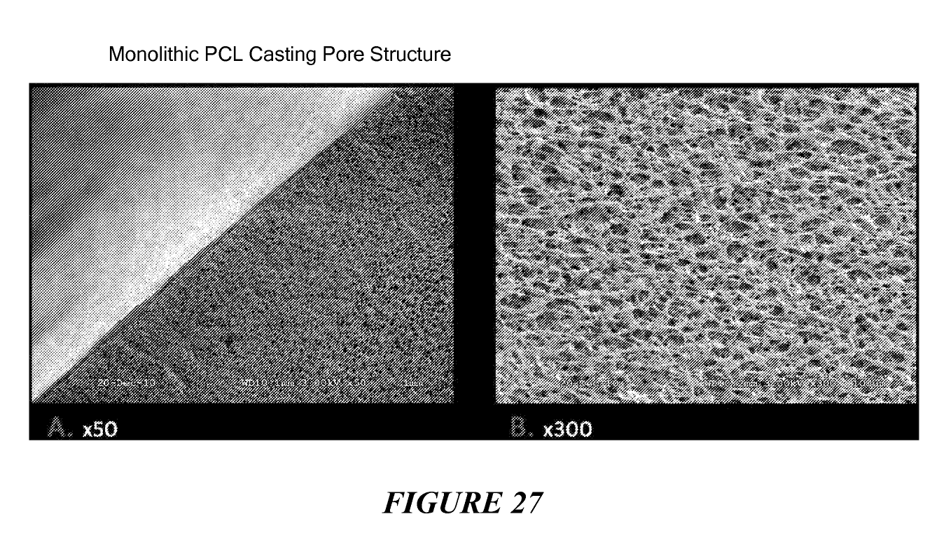

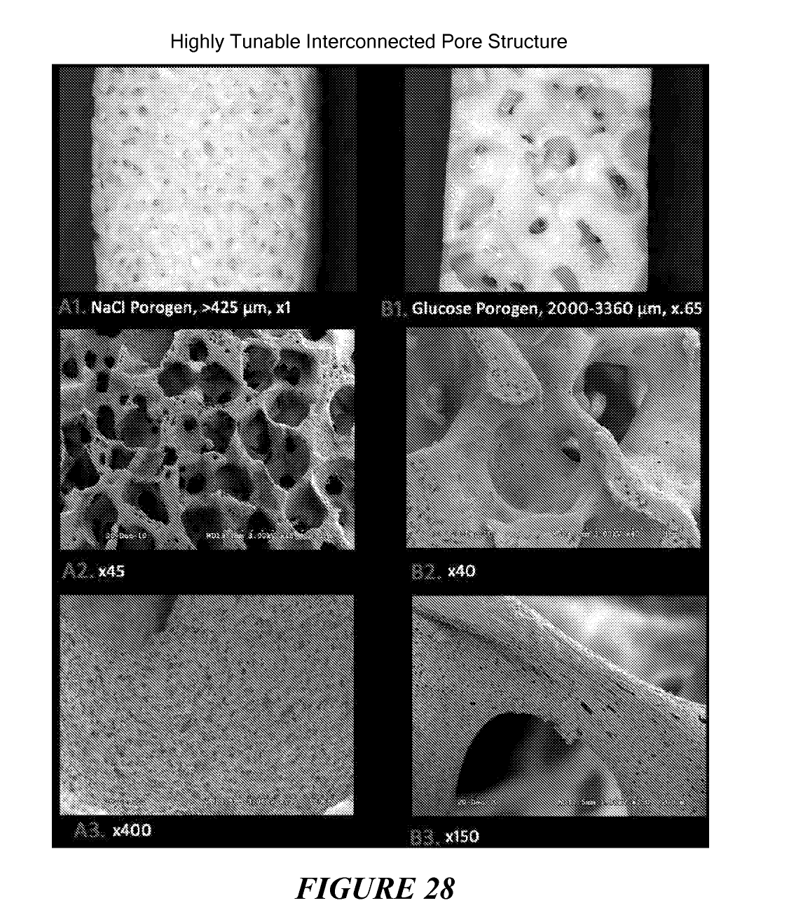

Samples of ProOsteon 500R™ were obtained from BioMet. Allograft fragments were coated in parallel with the ProOsteon fragments as previously described. Briefly, 10 kD PCL was dissolved in acetone at 60 mg/ml. Tobramycin was loaded into the formulation at 10% w/w of the PCL. Each fragment was dipped into the solution followed immediately by vacuum drying. The weight of the fragment was monitored after each coated to add an average analogous amount of weight to each fragment (−20 mg). After coating, tobramycin was allowed to release into PBS from each fragment or particulate aliquot for 6 weeks with the PBS being fully exchanged at each time point. Released tobramycin was assessed via ZOI studies (FIG. 26). There was no significant difference found between the synthetic and the allograft material.

11. Preparation of Highly Tunable Interconnected Pore Structure of Combination Biomaterial Substrates

Combination biomaterial substrates characterized by small pores that, in some instances display complete interconnectivity, and in other instances do not display complete interconnectivity, were prepared. The formulation and polymer coating microstructure are, in part, dependent on a non-solvent mixed phase. Water was investigated thoroughly as a non-solvent as well as other non-solvents such as ethanol, methanol, and combinations thereof. Each non-solvent could provide different utility and terminal structures. Freezing the structure at either −20° C. or −80° C. resulted in a high fidelity, low density polymer structure interstitial to the porogen network. Omitting the freezing step resulted in high density struts between individual porogen particles. Polymer concentrations in the 100 to 200 mg/mL range were used. It was also found that many values outside the 100 to 200 mg/mL range also yielded viable materials.

The combination of non-solvent structure combined with bulk porogen packing and/or stereo lithography techniques resulted in multiple levels of structural hierarchy, including foundational structural pores, larger secondary pores/channels, and overall gross structure. The high porosity of the resultant materials (typically >95%) produced a porous foam that is suited toward loading of a variety of therapeutics, including small molecule and protein drugs, extracellular matrix formulations, and cell suspensions. Loading of any of these therapeutics into an appropriate gel (collagen, HA, PEG, etc.) with subsequent infusion into the porous structure yielded highly homogenous distribution of both phases.

Hydrophobic drugs were easily taken up into the polymer during formulation and can be used in classical drug delivery approaches. Combinations of both solid loading and gel loading yielded potential multistage release platforms.

The combination biomaterial substrates can be subsequently modified with respect to their surface or bulk chemistry. Surface treatments include procedures such as plasma treatment to increase hydrophilicity or the grafting of bifunctional crosslinkers for the immobilization of biomolecules. Bulk treatments would include procedures such as oxidation to reduce the molecular weight of the polymer before implantation. In this fashion, a material could be fabricated at high molecular weight, chemically treated, and implanted at a reduced, application specific molecular weight

The degradation rate of the combination biomaterial substrate can be a function of polymer molecular weight; lower molecular weights are broken down (primarily via hydrolytic processes) and cleared faster than higher molecular weights. Degradation rates can be further assigned by blending multiple molecular weights at different ratios, or by solid state polymer matrix micro structure where pore volumes improve solvent accessibility to enhance hydrolysis. Mechanical properties are also a function of molecular weight, whereby higher KD PCL foams display more robust tensile and handling properties (i.e., more suturability, weldability, etc).

The combination biomaterial substrate can be used as a drug delivery wrap device, a tissue engineering scaffold, or for other applications. Applications also exist for in vitro assays that require a high surface area 3D substrate (organ culture, drug screening, bioreactor cultures, and advances cell cultures.

These combination biomaterial substrate formulations can be cast into large bulk blocks or sheets and trimmed into the desired shape (blocks, strips, various other polygonal and geometric forms). These formulations can also be cast into standard injection molds for more complicated monolithic shapes. Again, it can also be seamlessly integrated into stereolithography platforms for the CAD driven fabrication of highly complex and spatially heterogeneous structures.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A combination biomaterial comprising a mixture of (i) a substrate that is not collagen and (ii) a degradable polymer admixed with an agent that is bioactive or pharmaceutically active, wherein (a) the degradable polymer has a structure and a molecular weight selected to degrade over a time period when implanted within a subject so as to release the agent over the time period and (b) the substrate is a predominant component of the combination biomaterial.

2. The combination biomaterial of claim 1, wherein the degradable polymer has a structure and a molecular weight selected to degrade over a time period when implanted within a subject so as to release the agent in a therapeutically effective amount.

3. The combination biomaterial of claim 1, wherein the time period is greater than four weeks.

4. The combination biomaterial of claim 1, wherein the time period is greater than six weeks.

5. The combination biomaterial of claim 1, wherein the time period of greater than eight weeks.

6. The combination biomaterial of claim 1, wherein the release is a sustained release or an intermittent release.

7. The combination biomaterial of claim 1, wherein the substrate is selected from the group consisting of an autograft bone material, an alloplastic material, an allograft bone material, a demineralized bone matrix (DBM), a xenograft bone fragment, a calcium phosphate, a calcium sulfate, a calcium hydroxyphosphate, a hydroxyapatite, a purified coral, and composites thereof with a polymer, titanium, stainless steel, cobalt-chrome, or a tantalum.

8. The combination biomaterial of claim 1, wherein the substrate comprises morselized bone powder.

9. The combination biomaterial of claim 1, wherein the substrate comprises mineral components of bone.

10. The combination biomaterial of claim 1, wherein the degradable polymer has a molecular weight from about 5 kD to about 100 kD.

11. The combination biomaterial of claim 1, wherein the substrate is an indwelling medical device.

12. The combination biomaterial of claim 1, wherein the substrate is impregnated with the degradable polymer.

13. The combination biomaterial of claim 1, wherein a surface of the substrate is coated with one or more layers of the degradable polymer.

14. The combination biomaterial of claim 1, wherein the substrate is a porous matrix or a non-porous matrix of a degradable polymer.

15. The combination biomaterial of claim 1, wherein the degradable polymer is selected from the group consisting of polyglycolide (PGA), polylactic acid (PLA), poly(lactic-co-glycolic acid) (PLGA), polycaprolactone (PCL), polyurethane (PU), poly ethylene glycol (PEG), polyanhydrides, polyphosphazenes, resorbable polycarbonates, and any blend or copolymer thereof.

16. The combination biomaterial of claim 1, wherein the degradable polymer is selected from the group consisting of polyamino acids, polytyrosine, silk, recombinant poly amino acids, synthetic poly amino acids, proteins, fibrin, and albumin.

17. The combination biomaterial of claim 1, wherein the agent is microencapsulated in microspheres or nanoencapsulated in nanospheres.

18. The combination biomaterial of claim 1, wherein the agent comprises a biologically active excipient.

19. The combination biomaterial of claim 1, wherein the agent is encapsulated within, by, in, or inside the degradable polymer.

20. The combination biomaterial of claim 1, wherein the agent is selected from the group consisting of a growth factor, a therapeutic peptide, an antibody, a small molecule, a neovascular promoting agent, a polynucleotide, an anti-inflammatory agent, a chemo therapeutic agent, an anti-thrombogenic agent, an anticoagulant agent, and an analgesic agent.

21. The combination biomaterial of claim 1, wherein the agent is selected from the group consisting of an anti-infective agent, an antimicrobial agent, an antifungal agent, an antiviral agent, an antiseptic agent, a microcidal agent, and a bacteriostatic agent.

22. The combination biomaterial of claim 1, wherein the degradable polymer comprises monomer residues, wherein at least about 50% of the monomer residues have a structure represented by a formula: 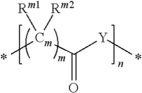
wherein m is an integer from 1 to 12;
wherein n is an integer selected to yield a molecular weight of the polymer of from about 5 kD to about 100 kD;
wherein Y is O or N—R, wherein R is hydrogen, optionally substituted alkyl, or optionally substituted aryl; and
wherein each of R m1 and R m2 is independently hydrogen, halogen, hydroxyl, nitrile, nitro, thiol, optionally substituted amino, and optionally substituted organic residue.

23. The combination biomaterial of claim 1, wherein the degradable polymer is a polyester.

24. The combination biomaterial of claim 1, wherein the degradable polymer is polycaprolactone.

25. The combination biomaterial of claim 1, wherein the degradable polymer is produced by a method comprising:
a) dissolving a starting polymer in a solution of a solvent at a concentration between 1 and 1000 mg/mL;
b) heating the solution to a temperature below the boiling point of the solvent to form a heated solvent solution;
c) adding a nonsolvent to the heated solvent solution to form a heated solvent/nonsolvent solution; and
d) reducing the temperature of the heated solvent/nonsolvent solution to induce a thermodynamic phase inversion so as to produce the degradable polymer.

26. The combination biomaterial of claim 25, wherein the solvent is acetone, ethyl acetate, or water.

27. The combination biomaterial of claim 25, wherein dissolving the starting polymer includes allowing the starting polymer to dissolve completely in the solvent.

28. The combination biomaterial of claim 25, wherein the nonsolvent is selected from the group consisting of water, ethanol, methanol, b-butanol, n-propanol, and isopropanol.

29. The combination biomaterial of claim 25, wherein adding the nonsolvent includes completely or partially dissolving the nonsolvent in the heated solvent/nonsolvent solution.

30. The combination biomaterial of claim 25, wherein the nonsolvent is water, and wherein the volume to volume percentage of water to solvent is 1 to 20%.

31. The combination biomaterial of claim 25, wherein the nonsolvent is ethanol, and wherein the volume to volume percentage of nonsolvent to solvent is 1 to 80%.

32. The combination biomaterial of claim 25, wherein the nonsolvent is methanol, and wherein the volume to volume percentage of nonsolvent to solvent of is 1 to 50%.

33. The combination biomaterial of claim 25, wherein the volume to volume ratio of nonsolvent to solvent is approximately 1:1.

34. The combination biomaterial of claim of 25, wherein the degradable polymer is produced by a method further comprising centrifuging the heated solvent/nonsolvent solution.

35. The combination biomaterial of claim 25, wherein the degradable polymer is produced by a method further comprising adding a solid particulate soluble porogen to the heated solvent/nonsolvent solution.

36. The combination biomaterial of claim 35, wherein the degradable polymer is produced by a method further comprising incorporating the solid particulate soluble porogen into the degradable polymer to create a secondary porous network within a phase-inverted microstructure.

37. The combination biomaterial of claim 35, wherein the solid particulate soluble porogen is selected from the group consisting of a metal chloride salt, a phosphate salt, a glucose, an alginate, an agar, a polyethylene glycol (PEG), a wax, and a gelatin.

38. The combination biomaterial of claim 35, wherein the solid particulate soluble porogen is selected from the group consisting of a metal gluconate salt and a nitrate salt.

39. The combination biomaterial of claim 35, wherein the degradable polymer is produced by a method further comprising removing the solid particulate soluble porogen from the degradable polymer.

40. The combination biomaterial of claim 39, wherein the degradable polymer is produced by a method further comprising removing the solid particulate soluble porogen from the degradable polymer using solvent extraction, thermal dissolution, or a combination thereof.

41. A combination biomaterial comprising:
a) a biocompatible, osteoconductive, porous substrate, wherein the substrate comprises a material selected from the group consisting of an allograft bone material, an augraft bone material, an alloplastic material, and a demineralized bone matrix (DBM), and wherein the substrate is not collagen; and
b) a degradable polymer admixed with an agent that is bioactive or pharmaceutically active, to form a polymer-agent mixture,
wherein the polymer has a structure and a molecular weight selected to degrade over a time period when implanted within a subject and thereby release the agent over the time period; and wherein the polymer-agent mixture is mixed with the substrate, forming a composite combination biomaterial comprised predominantly of substrate.

42. The combination biomaterial of claim 41, wherein the substrate is a demineralized bone matrix (DBM).
